SEQUENCE LISTING (77 sequences)

SEQ ID NO: 1 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 2 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 3 - protein, 5 aa, synthetic construct
YYGIS

SEQ ID NO: 4 - protein, 17 aa, synthetic construct
WISTYNGNTHYARMLQG

SEQ ID NO: 5 - protein, 15 aa, synthetic construct
ENYYGSGAYRGGMDV

SEQ ID NO: 6 - protein, 11 aa, synthetic construct
RASQSVSSYLA

SEQ ID NO: 7 - protein, 7 aa, synthetic construct
DASNRAT

SEQ ID NO: 8 - protein, 9 aa, synthetic construct
QQRSNWPWT

SEQ ID NO: 9 - protein, 453 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 454 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 10 aa, synthetic construct
GYDFTYYGIS

SEQ ID NO: 13 - protein, 30 aa, synthetic construct
QVQLVQSGAEVKKPGASVKVSCKASGYDFT

SEQ ID NO: 14 - protein, 14 aa, synthetic construct
WVRQAPGQGLEWMG

SEQ ID NO: 15 - protein, 32 aa, synthetic construct
RVTMTTDTSTRTAYMELRSLRSDDTAVYYCAR

SEQ ID NO: 16 - protein, 11 aa, synthetic construct
WGQGTTVTVSS

SEQ ID NO: 17 - protein, 23 aa, synthetic construct
EIVLTQSPATLSLSPGERATLSC

SEQ ID NO: 18 - protein, 15 aa, synthetic construct
WYQQKPGQAPRLLIY

SEQ ID NO: 19 - protein, 32 aa, synthetic construct
GIPARFSGSGSGTDFTLTISSLEPEDFAVYYC

SEQ ID NO: 20 - protein, 13 aa, synthetic construct
FGQGTKVEIKRTV

SEQ ID NO: 21 - protein, 25 aa, synthetic construct [sequence omitted]

SEQ ID NO: 22 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGSYRGGMDVWGQGTTVTVSS

SEQ ID NO: 23 - protein, 124 aa, synthetic construct
QVQLVQSGAEVKKPGASVKVSCKASGYDFTYYGISWVRQAPGQGLEWMGWISTYNGNKHYARMLQGRVTMTTDTSTRTAYMELRSLRSDDTAVYYCARENYYGSGSYRGGMDVWGQGTTVTVSS

SEQ ID NO: 24 - protein, 124 aa, synthetic construct
QVQLVQSGAEVKKPGASVKVSCKASGYDFTYYGISWVRQAPGQGLEWMGWISTYNGGTHYARMLQGRVTMTTDTSTRTAYMELRSLRSDDTAVYYCARENYYGSGSYRGGMDVWGQGTTVTVSS

SEQ ID NO: 25 - protein, 124 aa, synthetic construct
QVQLVQSGAEVKKPGASVKVSCKASGYDFTYYGISWVRQAPGQGLEWMGWISTYNGVTHYARMLQGRVTMTTDTSTRTAYMELRSLRSDDTAVYYCARENYYGSGSYRGGMDVWGQGTTVTVSS

SEQ ID NO: 26 - protein, 124 aa, synthetic construct
QVQLVQSGAEVKKPGASVKVSCKASGYDFTYYGISWVRQAPGQGLEWMGWISTYNGGTHYARMLQGRVTMTTDTSTRTAYMELRSLRSDDTAVYYCARENYYGSGAYRGGMDAWGQGTTVTVSS

SEQ ID NO: 27 - protein, 124 aa, synthetic construct
QVQLVQSGAEVKKPGASVKVSCKASGYDFTYYGISWVRQAPGQGLEWMGWISTYNGVTHYARMLQGRVTMTTDTSTRTAYMELRSLRSDDTAVYYCARENYYGSGAYRGGMDAWGQGTTVTVSS

SEQ ID NO: 28 - protein, 124 aa, synthetic construct
QVQLVQSGAEVKKPGASVKVSCKASGYDFTYYGISWVRQAPGQGLEWMGWISTYNGKTHYARMHQGRVTMTTDTSTRTAYMELRSLRSDDTAVYYCARENYYGSGAYRGGMDAWGQGTTVTVSS

SEQ ID NO: 29 - protein, 124 aa, synthetic construct
QVQLVQSGAEVKKPGASLKVSCKASGYPFTNYGISWVRQAPGQGLEWMGWISTYNGNTHYAQKLQGRVTMTTDTSTTTAYMDLRSLRSDDTAVYYCARENYYGSGSYRGGMDVWGQGTTVTVSS

SEQ ID NO: 30 - protein, 124 aa, synthetic construct
QVQLVQSGAEVKKPGASVKVSCKASGYSFTTYGISWVRQAPGQGLEWMGWISTYNGNTHYAQKLQGRVTMTTDTSTRTAYMELRSLRSDDTAVYYCARENYYGSGSYRGGMDVWGQGTTVTVSS

SEQ ID NO: 31 - protein, 121 aa, synthetic construct
QVQLVQSGAEVKKPGASVKVSCKASGYTFTSYGINWVRQAPGQGLEWMGWISTYNGNTNSAQKLQGRVTMTTDTSTSTAYMELRSLRSDDTAVYYCARAHSSSWFDAFDIWGQGTMVTVSS

SEQ ID NO: 32 - protein, 126 aa, synthetic construct
QVQLVESGGGVVQPGRSLRLSCAASGFTFSSFAMHWVRQAPGKGLEWVALIPFDGSSNYYADSVKGRFTISRDNSKNTLYLQMNSLRAEDTAVYYCARDRNYYGSGSFSFDAFDIWGQGTLVTVSS

SEQ ID NO: 33 - protein, 126 aa, synthetic construct
QVQLVESGGGVVQPGRSLRLSCAASGFTFSNYALHWVRQAPGKGLEWVALISYDGSDKYYADSVKGRFAISRDNSKNTLYLQMNSLRAEDTAVYYCARDREYCTYSSCSYDAFDIWGQGTMVTVSS

SEQ ID NO: 34 - protein, 121 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGYSSAWFDAFDIWGQGTMVTVSS

SEQ ID NO: 35 - protein, 107 aa, synthetic construct [sequence omitted]

SEQ ID NO: 36 - protein, 107 aa, synthetic construct
DIQMTQSPSSLSASVGDRVTITCRASQGISNWLAWYQQKPEKAPKSLIYAASSLQSGVPSRFSGSGSGTDFTLTISSLQPEDFATYYCQQYNSYPYTFGQGTKLEIK

SEQ ID NO: 37 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDYATYYCQQYNSYPYTFGQGTKLEIK

SEQ ID NO: 38 - protein, 107 aa, synthetic construct [sequence omitted]

SEQ ID NO: 39 - protein, 121 aa, synthetic construct
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 40 - protein, 106 aa, synthetic construct
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 41 - protein, 449 aa, synthetic construct
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 216 aa, synthetic construct
QSVLTQPPSVSGAPGQRVTISCTSSSSDIGAGLGVHWYQQLPGTAPKLLIYGYYNRPSGVPDRFSGSKSGTSASLTITGLLPEDEGDYYCQSYDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 43 - protein, 119 aa, synthetic construct
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 44 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 45 - protein, 446 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 30 aa, synthetic construct
QVQLVQSGAEVKKPGASVKVSCKASGFDIQ

SEQ ID NO: 47 - protein, 5 aa, synthetic construct
DTYMH

SEQ ID NO: 48 - protein, 14 aa, synthetic construct
WVKQRPGQGLEWMG

SEQ ID NO: 49 - protein, 17 aa, synthetic construct
RIDPASGHTKYDPKFQV

SEQ ID NO: 50 - protein, 32 aa, synthetic construct
RVTITRDTSTSTVYLELSSLRSEDTAVYYCAR

SEQ ID NO: 51 - protein, 7 aa, synthetic construct
SGGLPDV

SEQ ID NO: 52 - protein, 11 aa, synthetic construct
WGQGTTVTVSS

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 213 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 23 aa, synthetic construct
EIVLTQSPGTLSLSPGERATLSC

SEQ ID NO: 56 - protein, 10 aa, synthetic construct
RASSSVSYMY

SEQ ID NO: 57 - protein, 15 aa, synthetic construct
WYQQKPGQAPRPLIY

SEQ ID NO: 58 - protein, 7 aa, synthetic construct
ATSNLAS

SEQ ID NO: 59 - protein, 32 aa, synthetic construct
GIPDRFSGSGSGTDFTLTISRLEPEDFAVYYC

SEQ ID NO: 60 - protein, 9 aa, synthetic construct
QQWEGNPRT

SEQ ID NO: 61 - protein, 13 aa, synthetic construct
FGGGTKLEIKRTV

SEQ ID NO: 62 - protein, 104 aa, synthetic construct
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 64 - protein, 109 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTV

SEQ ID NO: 65 - protein, 10 aa, synthetic construct
GYTFTSYDIN

SEQ ID NO: 66 - protein, 10 aa, synthetic construct
WLNPNSGYTG

SEQ ID NO: 67 - protein, 10 aa, synthetic construct
EVPETAAFEY

SEQ ID NO: 68 - protein, 14 aa, synthetic construct
TSSSSDIGAGLGVH

SEQ ID NO: 69 - protein, 7 aa, synthetic construct
GYYNRPS

SEQ ID NO: 70 - protein, 10 aa, synthetic construct
QSWDGTLSAL

SEQ ID NO: 71 - protein, 119 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 72 - protein, 111 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 73 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 216 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 448 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0376530 A1
(43) Pub. Date: Dec. 11, 2025

(54) METHODS FOR TREATMENT OF INFLAMMATORY BOWEL DISEASE WITH AN ANTI-TL1A ANTIBODY

(71) Applicants: Genentech, Inc., South San Francisco, CA (US); Hoffmann-La Roche Inc., Little Falls, NJ (US); Pfizer Inc., New York, NY (US)

(72) Inventors: Akiko CHAI, San Carlos, CA (US); Karen LASCH, Wilmette, IL (US); Kun Tae PARK, Sunnyvale, CA (US); Daniela BOJIC, Basel (CH); Pascal ESPIE, Saint Louis (FR)

(21) Appl. No.: 19/210,817

(22) Filed: May 16, 2025

Related U.S. Application Data
(60) Provisional application No. 63/649,290, filed on May 17, 2024.
(60) Provisional application No. 63/727,956, filed on Dec. 4, 2024.
(60) Provisional application No. 63/773,082, filed on Mar. 17, 2025.

(51) Int. Cl. C07K 16/28; A61K 39/00

(52) U.S. Cl. CPC C07K 16/2875; A61K 2039/545; C07K 2317/565

(57) ABSTRACT

The present disclosure provides methods and compositions for treating inflammatory bowel disease (IBD), e.g., ulcerative colitis (UC) or Crohn's disease (CD), with a therapeutic dose of an anti-TNF-like ligand 1A (TL1A) antibody.

### SEQUENCE LISTING

The instant application contains a Sequence Listing which has been submitted electronically in XML format and is hereby incorporated by reference in its entirety. Said XML copy, created on May 13, 2025, is named 50474-353005_Sequence_Listing_5_13_25 and is 75,543 bytes in size.

### TECHNICAL FIELD

The present invention relates to the treatment of signs and symptoms of inflammatory bowel disease (IBD), e.g., ulcerative colitis (UC) or Crohn's disease (CD), with an anti-tumor necrosis factor-like ligand 1A (TL1A) antibody.

### BACKGROUND OF THE INVENTION

Inflammatory bowel disease (IBD), which encompasses Crohn's disease and ulcerative colitis (UC), is a chronic inflammatory disorder affecting about 1.6 million people in the USA, and about 2.5-3 million people in Europe.

The aim of medical treatment in IBD is to control inflammation and reduce symptoms. Available treatment options for moderately to severely active UC include appropriate doses of oral corticosteroids; biologic therapies such as the tumor necrosis factor inhibitors (TNFi) infliximab, adalimumab, ustekinumab, and golimumab; the integrin receptor antagonist vedolizumab; and the orally administered small molecule Janus kinase inhibitor tofacitinib. However, non-response, inadequate response, and loss of response to these and other treatments are known to occur, and these treatments may have significant adverse effects. Therefore, the development of novel treatments for IBD (e.g., UC and CD) remains an unmet clinical need.

One of the immune components involved in the pathogenesis of IBD is TNF-like ligand 1A (TL1A, also known as tissue necrosis factor superfamily member 15 (TNFSF15)). Genome-wide association studies have linked TNFSF15 single-nucleotide polymorphisms (SNPs) with disease severity; for example, an association was observed between the rs11554257 SNP and medically refractory UC compared with healthy controls (Haritunians et al., Inflammatory Bowel Diseases; 16:1830-1840, 2010). TL1A has been found to be upregulated in IBD tissue specimens, with level of expression corresponding to the severity of disease (Bamias et al., Clin Immunol; 137:242-249, 2010). TL1A promotes inflammation and intestinal fibrosis in IBD. Further, variants in TNFSF15 have been linked to the pathogenesis of several autoimmune diseases, including psoriasis, rheumatoid arthritis, and multiple sclerosis, implicating a broad role for TNFSF15 in human inflammatory diseases.

Binding of TL1A to its receptor death receptor 3 (DR3) stimulates T cell-mediated signaling and cytokine production. Because increased cytokine production leads to chronic inflammation, inhibition of TL1A is a promising therapeutic strategy for treatment of inflammatory diseases, including IBD.

Thus, there is a need in the art for methods of treating inflammatory bowel disease, including Crohn's disease and ulcerative colitis, with anti-TNF-like ligand 1A (TL1A) antibody therapy. The present disclosure addresses these needs.

### SUMMARY OF THE INVENTION

In one aspect, the disclosure provides a method of treating an inflammatory bowel disease (IBD) in a patient, the method comprising administering to the patient an effective amount of an anti-TNF-like ligand 1A (TL1A) antibody in a dosing regimen comprising an induction phase, wherein the induction phase comprises administration of only four doses of the anti-TL1A antibody, wherein (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose, wherein the anti-TL1A antibody comprises the following complementarity-determining regions (CDRs): (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the dosing regimen further comprises a maintenance phase.

In another aspect, the disclosure provides a method of treating an IBD in a patient, the method comprising administering to the patient an effective amount of an anti-TL1A antibody in a dosing regimen comprising an induction phase and a maintenance phase, wherein (a) the induction phase comprises administration of only four doses of the anti-TL1A antibody, wherein (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the maintenance phase comprises administration of the anti-TL1A antibody every four weeks, wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects, in the induction phase, the anti-TL1A antibody is administered intravenously at a dose of about 500 mg.

In some aspects, in the induction phase, the anti-TL1A antibody is administered subcutaneously at a dose of about 500 mg.

In some aspects, the induction phase has a duration of about 12 weeks. In some aspects, (a) the first dose of the anti-TL1A antibody is administered on about Day 1 of Week 0; (b) the second dose of the anti-TL1A antibody is administered on about Day 1 of Week 2; (c) the third dose of the anti-TL1A antibody is administered on about Day 1 of Week 6; and (d) the fourth dose of the anti-TL1A antibody is administered on about Day 1 of Week 10.

In some aspects, in the maintenance phase, the anti-TL1A antibody is administered subcutaneously at a dose of about 150 mg. In some aspects, in the maintenance phase, the anti-TL1A antibody is administered subcutaneously at a dose of about 450 mg.

In some aspects, the maintenance phase comprises at least two doses of the anti-TL1A antibody.

In some aspects, the maintenance phase comprises subcutaneous administration of the anti-TL1A antibody every four weeks. In some aspects, the maintenance phase comprises subcutaneous administration of the anti-TL1A antibody every two weeks. In some aspects, the maintenance phase comprises (i) at least one interval in which the anti-TL1A antibody is administered every four weeks and (ii) at least one interval in which the anti-TL1A antibody is administered every two weeks.

In some aspects, the maintenance phase comprises eleven doses of the anti-TL1A antibody.

In some aspects, the maintenance phase has a duration of about 40 weeks.

In some aspects in which the method comprises a maintenance phase, a corticosteroid is administered to the patient daily in the induction phase, and the dose of the corticosteroid is tapered during the maintenance phase. In some aspects, administration of the corticosteroid is discontinued during the maintenance phase.

In some aspects, the corticosteroid is prednisone or an equivalent thereof, budesonide, or budesonide multi-matrix (MMX). In some aspects, the corticosteroid is prednisone or an equivalent thereof and was administered to the patient orally at a dose of more than 10 mg per day in the induction phase, and the tapering comprises (i) tapering the dose by 5 mg per week until the patient is receiving a dose of 10 mg per day; and (ii) tapering the dose by 2.5 mg per week until a dose of 0 mg per week is reached. In some aspects, the corticosteroid is prednisone or an equivalent thereof and was administered to the patient orally at a dose 10 mg or less per day in the induction phase, and the tapering comprises tapering the dose by 2.5 mg per week until a dose of 0 mg per week is reached. In some aspects, the corticosteroid is budesonide or budesonide MMX and was administered to the patient orally at a dose of 9 mg or less per day in the induction phase, and the tapering comprises (i) administering the corticosteroid at a dose of 9 mg every other day for two weeks; (ii) administering the corticosteroid at a dose of 9 mg every third day for two weeks; and (iii) discontinuing administration of the corticosteroid.

In some aspects in which the method comprises a maintenance phase, the first dose of the maintenance phase is administered about two weeks after administration of the fourth dose of the induction phase.

In some aspects, the dosing regimen has a duration of about 52 weeks. In some aspects, (a) the first dose of the induction phase is administered on about Day 1 of Week 0; (b) the second dose of the induction phase is administered on about Day 1 of Week 2; (c) the third dose of the induction phase is administered on about Day 1 of Week 6; (d) the fourth dose of the induction phase is administered on about Day 1 of Week 10; (e) the first dose of the maintenance phase is administered on about Day 1 of Week 12; and (f) the subsequent doses of the maintenance phase are administered on about Day 1 of Weeks 16, 20, 24, 28, 32, 36, 40, 44, 48, and 52.

In some aspects, the dosing regimen further comprises an extension phase comprising administration of one or more additional dosing cycles of the anti-TL1A antibody. In some aspects, (a) the extension phase comprises subcutaneous administration of the anti-TL1A antibody every four weeks; (b) the extension phase comprises subcutaneous administration of the anti-TL1A antibody every two weeks; or (c) the extension phase comprises (i) at least one interval in which the anti-TL1A antibody is administered every four weeks and (ii) at least one interval in which the anti-TL1A antibody is administered every two weeks. In some aspects, the one or more dosing cycles of the extension phase are administered to the patient following disease worsening during the maintenance phase.

In some aspects, the IBD is ulcerative colitis (UC). In some aspects, the UC is moderately to severely active ulcerative colitis. In some aspects, the patient has a modified Mayo score (mMS) of between 5 points and 9 points. In some aspects, the patient has a Mayo endoscopic score(ES) of 2 or 3.

In some aspects, the IBD is Crohn's disease (CD). In some aspects, the CD is moderately to severely active CD. In some aspects, (a) the patient has a Simple Endoscopic Score for Crohn's Disease (SES-CD) of equal to or greater than 6; or (b) the patient has isolated ileal disease only, and has an SES-CD of equal to or greater than 4. In some aspects, the patient has a Crohn's disease activity index (CDAI) that is at least 220 and is no greater than 450.

In some aspects, the patient has previously been treated with a therapy for UC or CD and has experienced inadequate response to the therapy, loss of response to the therapy, and/or intolerance of the therapy.

In some aspects, the therapy was a conventional therapy. In some aspects, the conventional therapy comprised administration of a steroid, an immunomodulator, or an oral aminosalicylate.

In some aspects, the therapy was an advanced therapy. In some aspects, the advanced therapy comprised administration of an anti-tumor necrosis factor (TNF) agent, an anti-integrin agent, an anti-IL12/IL23 agent, a Janus kinase (JAK) inhibitor, or a sphingosine-1-phosphate (S1P) inhibitor.

In another aspect, the disclosure provides a method of treating ulcerative colitis (UC) in a patient, the method comprising administering to the patient an effective amount of an anti-TNF-like ligand 1A (TL1A) antibody in a dosing regimen comprising an induction phase and a maintenance phase, wherein (a) the induction phase comprises intravenous administration of only four doses of the anti-TL1A antibody at a dose of about 500 mg, wherein (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the maintenance phase comprises subcutaneous administration of the anti-TL1A antibody every four weeks at a dose of about 450 mg, wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects in which the patient has UC, in a population of patients treated according to the method, the treating results in an increase in the proportion of patients who have achieved clinical remission at the end of the induction phase as compared to a reference population. In some aspects, the induction phase has a duration of about 12 weeks, and the treating results in an increase in the proportion of patients who have achieved clinical remission at Week 12.

In some aspects in which the patient has UC, in a population of patients treated according to the method, the treating results in an increase in the proportion of patients who have achieved clinical remission at the end of the maintenance phase as compared to a reference population. In some aspects, the dosing regimen has a duration of about 52 weeks, and the treating results in an increase in the proportion of patients who have achieved clinical remission at Week 52.

In some aspects in which the patient has UC, clinical remission is a modified Mayo Score (mMS)≤2 with stool frequency subscore (SFS)=0 or 1, rectal bleeding subscore (RBS)=0, and endoscopic subscore(ES)=0 or 1.

In another aspect, the disclosure provides a method of treating Crohn's disease (CD) in a patient, the method comprising administering to the patient an effective amount of an anti-TNF-like ligand 1A (TL1A) antibody in a dosing regimen comprising an induction phase and a maintenance phase, wherein (a) the induction phase comprises intravenous administration of only four doses of the anti-TL1A antibody at a dose of about 500 mg, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the maintenance phase comprises subcutaneous administration of the anti-TL1A antibody every four weeks at a dose of about 450 mg, wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides a method of treating CD in a patient, the method comprising administering to the patient an effective amount of an anti-TL1A antibody in a dosing regimen comprising an induction phase and a maintenance phase, wherein (a) the induction phase comprises intravenous administration of only four doses of the anti-TL1A antibody at a dose of about 500 mg, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the maintenance phase comprises subcutaneous administration of the anti-TL1A antibody every four weeks at a dose of about 150 mg, wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects in which the patient has CD, in a population of patients treated according to the method, the treating results in an increase in the proportion of patients who have achieved clinical remission at the end of the induction phase as compared to a reference population. In some aspects, the induction phase has a duration of about 12 weeks, and the treating results in an increase in the proportion of patients who have achieved clinical remission at Week 12.

In some aspects in which the patient has CD, a population of patients treated according to the method, the treating results in an increase in the proportion of patients who have achieved clinical remission at the end of the maintenance phase as compared to a reference population. In some aspects, the dosing regimen has a duration of about 52 weeks, and the treating results in an increase in the proportion of patients who have achieved clinical remission at Week 52.

In some aspects in which the patient has CD, clinical remission is a Crohn's disease activity index (CDAI) of less than 150.

In some aspects in which the patient has CD, in a population of patients treated according to the method, the treating results in an increase in the proportion of patients who have achieved an endoscopic response at the end of the induction phase as compared to a reference population. In some aspects, the induction phase has a duration of about 12 weeks, and the treating results in an increase in the proportion of patients who have achieved an endoscopic response at Week 12.

In some aspects in which the patient has CD, in a population of patients treated according to the method, the treating results in an increase in the proportion of patients who have achieved an endoscopic response at the end of the maintenance phase as compared to a reference population. In some aspects, the dosing regimen has a duration of about 52 weeks, and the treating results in an increase in the proportion of patients who have achieved an endoscopic response at Week 52.

In some aspects in which the patient has CD, an endoscopic response is a Simple Endoscopic Score for Crohn's Disease (SES-CD) that is at least 50% lower than a baseline SES-CD.

In some aspects, the reference population is a population of patients who have not been treated with an anti-TL1A antibody. In some aspects, the reference population is a population of patients who have been treated with a placebo.

In some aspects, the patient has been determined to be a haplotype B non-carrier for TNFSF15.

In some aspects, the anti-TL1A antibody comprises (a) a heavy chain variable (VH) domain comprising an amino acid sequence having at least 95% sequence identity to the amino acid sequence of SEQ ID NO: 1; and/or (b) a light chain variable (VL) domain comprising an amino acid sequence having at least 95% sequence identity to the amino acid sequence of SEQ ID NO: 2.

In some aspects, the anti-TL1A antibody comprises (a) a VH domain comprising the amino acid sequence of SEQ ID NO: 1; and/or (b) a VL domain comprising the amino acid sequence of SEQ ID NO: 2. In some aspects, the anti-TL1A antibody comprises (a) a VH domain comprising the amino acid sequence of SEQ ID NO: 1; and (b) a VL domain comprising the amino acid sequence of SEQ ID NO: 2.

In some aspects, the anti-TL1A antibody comprises (a) a heavy chain comprising an amino acid sequence having at least 95% sequence identity to the amino acid sequence of SEQ ID NO: 9 or SEQ ID NO: 11; and/or (b) a light chain comprising an amino acid sequence having at least 95% sequence identity to the amino acid sequence of SEQ ID NO: 10. In some aspects, the anti-TL1A antibody comprises (a) a heavy chain comprising the amino acid sequence of SEQ ID NO: 9; and/or (b) a light chain comprising the amino acid sequence of SEQ ID NO: 10. In some aspects, the anti-TL1A antibody comprises (a) a heavy chain comprising the amino acid sequence of SEQ ID NO: 9; and (b) a light chain comprising the amino acid sequence of SEQ ID NO: 10.

In some aspects, the anti-TL1A antibody is afimkibart.

In another aspect, the disclosure provides a method of treating an inflammatory bowel disease (IBD) in a patient, the method comprising administering to the patient an effective amount of an anti-TNF-like ligand 1A (TL1A) antibody in a dosing regimen comprising an induction phase, wherein the induction phase comprises administration of only four doses of the anti-TL1A antibody, wherein (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises: (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10. In some aspects, the IBD is UC. In some aspects, the IBD is CD.

In another aspect, the disclosure provides a method of treating an IBD in a patient, the method comprising administering to the patient an effective amount of an anti-TL1A antibody in a dosing regimen comprising an induction phase and a maintenance phase, wherein (a) the induction phase comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the maintenance phase comprises administration of the anti-TL1A antibody every four weeks; wherein the anti-TL1A antibody comprises: (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10. In some aspects, the IBD is UC. In some aspects, the IBD is CD.

In some aspects, the patient is a human.

In some aspects, the anti-TL1A antibody is administered in combination with one or more additional therapeutic agents.

In another aspect, the disclosure provides a kit comprising an anti-TNF-like ligand 1A (TL1A) antibody and a package insert comprising instructions for using the antibody for treating an inflammatory bowel disease (IBD) in a patient in need thereof according to any of the methods provided herein.

In another aspect, the disclosure provides an anti-TL1A antibody for use in treating IBD in a patient, wherein an effective amount of the anti-TL1A antibody is to be administered to the patient in a dosing regimen comprising an induction phase, wherein the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose, and wherein the anti-TL1A antibody comprises the following complementarity-determining regions (CDRs): (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides an anti-TL1A antibody for use in treating an IBD in a patient, wherein an effective amount of the anti-TL1A antibody is to be administered to the patient in a dosing regimen comprising an induction phase and a maintenance phase, wherein (a) the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises administration of the anti-TL1A antibody every four weeks, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides an anti-TL1A antibody for use in treating UC in a patient, wherein an effective amount of an anti-TL1A antibody is to be administered to the patient in a dosing regimen comprising an induction phase and a maintenance phase, wherein (a) the induction phase comprises intravenous administration of only four doses of the anti-TL1A antibody at a dose of about 500 mg, wherein: (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises subcutaneous administration of the anti-TL1A antibody every four weeks at a dose of about 450 mg, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides an anti-TL1A antibody for use in treating CD in a patient, wherein an effective amount of an anti-TL1A antibody is to be administered to the patient in a dosing regimen comprising an induction phase and a maintenance phase, wherein (a) the induction phase comprises intravenous administration of only four doses of the anti-TL1A antibody at a dose of about 500 mg, wherein: (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises subcutaneous administration of the anti-TL1A antibody every four weeks at a dose of about 450 mg, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides an anti-TL1A antibody for use in treating CD in a patient, wherein an effective amount of an anti-TL1A antibody is to be administered to the patient in a dosing regimen comprising an induction phase and a maintenance phase, wherein (a) the induction phase comprises intravenous administration of only four doses of the anti-TL1A antibody at a dose of about 500 mg, wherein: (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises subcutaneous administration of the anti-TL1A antibody every four weeks at a dose of about 150 mg, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides an anti-TL1A antibody for use in treating IBD in a patient, wherein an effective amount of an anti-TL1A antibody is to be administered to the patient in a dosing regimen comprising an induction phase, wherein the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose, and wherein the anti-TL1A antibody comprises (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10.

In another aspect, the disclosure provides an anti-TL1A antibody for use in treating IBD in a patient, wherein an effective amount of an anti-TL1A antibody is to be administered to the patient in a dosing regimen comprising an induction phase and a maintenance phase, wherein (a) the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises administration of the anti-TL1A antibody every four weeks, and wherein the anti-TL1A antibody comprises (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10.

In another aspect, the disclosure provides use of an anti-TL1A antibody in the manufacture of a medicament for treating IBD in a patient, wherein an effective amount of the anti-TL1A antibody is to be administered to the patient in a dosing regimen comprising an induction phase, wherein the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose, and wherein the anti-TL1A antibody comprises the following complementarity-determining regions (CDRs): (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides use of an anti-TL1A antibody in the manufacture of a medicament for treating an IBD in a patient, wherein an effective amount of the anti-TL1A antibody is to be administered to the patient in a dosing regimen comprising an induction phase and a maintenance phase, wherein (a) the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises administration of the anti-TL1A antibody every four weeks, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides use of an anti-TL1A antibody in the manufacture of a medicament for treating UC in a patient, wherein an effective amount of an anti-TL1A antibody is to be administered to the patient in a dosing regimen comprising an induction phase and a maintenance phase, wherein (a) the induction phase comprises intravenous administration of only four doses of the anti-TL1A antibody at a dose of about 500 mg, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises subcutaneous administration of the anti-TL1A antibody every four weeks at a dose of about 450 mg, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides use of an anti-TL1A antibody in the manufacture of a medicament for treating CD in a patient, wherein an effective amount of an anti-TL1A antibody is to be administered to the patient in a dosing regimen comprising an induction phase and a maintenance phase, wherein (a) the induction phase comprises intravenous administration of only four doses of the anti-TL1A antibody at a dose of about 500 mg, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises subcutaneous administration of the anti-TL1A antibody every four weeks at a dose of about 450 mg, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides use of an anti-TL1A antibody in the manufacture of a medicament for treating CD in a patient, wherein an effective amount of an anti-TL1A antibody is to be administered to the patient in a dosing regimen comprising an induction phase and a maintenance phase, wherein (a) the induction phase comprises intravenous administration of only four doses of the anti-TL1A antibody at a dose of about 500 mg, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises subcutaneous administration of the anti-TL1A antibody every four weeks at a dose of about 150 mg, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In another aspect, the disclosure provides use of an anti-TL1A antibody in the manufacture of a medicament for treating IBD in a patient, wherein an effective amount of an anti-TL1A antibody is to be administered to the patient in a dosing regimen comprising an induction phase, wherein the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose, and wherein the anti-TL1A antibody comprises (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10.

In another aspect, the disclosure provides use of an anti-TL1A antibody in the manufacture of a medicament for treating IBD in a patient, wherein an effective amount of an anti-TL1A antibody is to be administered to the patient in a dosing regimen comprising an induction phase and a maintenance phase, wherein (a) the induction phase comprises administration of only four doses of the anti-TL1A antibody, wherein (i) the second dose of the anti-TL1A antibody is to be administered about two weeks after the first dose; (ii) the third dose of the anti-TL1A antibody is to be administered about four weeks after the second dose; and (iii) the fourth dose of the anti-TL1A antibody is to be administered about four weeks after the third dose; and (b) the maintenance phase comprises administration of the anti-TL1A antibody every four weeks, and wherein the anti-TL1A antibody comprises (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10.

### BRIEF DESCRIPTION OF THE DRAWINGS

FIG. 1 is a schematic diagram showing the design of the GA45329 study. IV=intravenous; SC=subcutaneous; BL=baseline; D=day; OLE=open-label extension; W=week; Q2W=every 2 weeks; Q4W=every 4 weeks; R=randomization.

FIG. 2A is a schematic diagram showing the design of the open-label extension phase of the GA45329 study. DW=disease worsening; DWC=disease worsening criteria; OLE=open-label extension; W=week; Q2W=every 2 weeks; Q4W=every 4 weeks.

FIG. 2B is a schematic diagram showing the design of the open-label extension phase of the GA45330 study. DW=disease worsening; DWC=disease worsening criteria; W12=Week 12; Q2W=every 2 weeks; Q4W=every 4 weeks.

FIG. 3 is a schematic diagram showing the design of the GA45330 study. IV=intravenous; SC=subcutaneous; BL=baseline; D=day; OLE=open-label extension; W=week; Q2W=every 2 weeks; Q4W=every 4 weeks; R=randomization.

FIG. 4 is a schematic diagram showing the design of the GA45331 study. IV=intravenous; SC=subcutaneous; BL=baseline; D=day; OLE=open-label extension; W=week; Q2W=every 2 weeks; Q4W=every 4 weeks; R=randomization.

FIG. 5 is a schematic diagram showing the design of the GA45332 study. IV=intravenous; SC=subcutaneous; BL=baseline; D=day; OLE=open-label extension; W=week; Q2W=every 2 weeks; Q4W=every 4 weeks; R=randomization.

FIG. 6 is a schematic diagram showing the design of the open-label extension (OLE) phase of the GA45331 study. DW=disease worsening; DWC=disease worsening criteria; SC=subcutaneous; W=week.

FIG. 7 is a schematic diagram showing the design of the open-label extension (OLE) phase of the GA45332 study. DW=disease worsening; DWC=disease worsening criteria; SC=subcutaneous; W=week.

FIG. 8 is a schematic diagram showing a comparison of the Phase 3 GA45329 (ulcerative colitis (UC)) and GA45331 (Crohn's disease (CD)) studies to the Phase 2a (TUSCANY) and Phase 2b (TUSCANY-2) trials. The 12-week cumulative bioavailable dose (in mg) is shown. IV=intravenous; SC=subcutaneous; OL=open-label; Q4W=every 4 weeks; f-u=follow-up; mo=months.

FIG. 9 is a set of graphs showing total pharmacokinetics (PK) (total drug concentration in the serum (in μg/mL)) of afimkibart in the induction phase dosing regimen of the Phase 2a (TUSCANY) trial (Ph2a (UC)), the 450 mg induction phase dosing regimen of the Phase 2b (TUSCANY-2) trial (Ph2B (UC)), and two induction phase dosing regimens for the Phase 3 trials provided herein over time. 4×IV: afimkibart is administered intravenously (IV) at Weeks 0, 2, 6, and 10 of a 12-week dosing regimen, for a total of four doses. 5×IV: afimkibart is administered IV at Weeks 0, 2, 4, 6, and 10 of a 12-week dosing regimen, for a total of five doses. The Phase 2a and Phase 2b plots present real data; the Phase 3 plots are modeled. In the modeled panels, solid lines show the median drug concentration, and dotted lines show the 90% confidence interval (CI).

FIG. 10 is a set of graphs showing levels of soluble TL1A (sTL1A) (in pg/mL) in the induction phase dosing regimen of the Phase 2a (TUSCANY) trial (Ph2a), the 450 mg induction phase dosing regimen of the Phase 2b (TUSCANY-2) trial (Ph2B), and two induction phase dosing regimens for the Phase 3 trials provided herein over time. 4×IV: afimkibart is administered intravenously (IV) at Weeks 0, 2, 6, and 10 of a 12-week dosing regimen, for a total of four doses. 5×IV: afimkibart is administered IV at Weeks 0, 2, 4, 6, and 10 of a 12-week dosing regimen, for a total of five doses. The Phase 2a and Phase 2b plots present real data; the Phase 3 plots are modeled.

### DETAILED DESCRIPTION OF THE INVENTION

The present invention provides therapeutic methods and compositions for treatment of inflammatory bowel disease, e.g. ulcerative colitis (UC) and Crohn's disease (e.g., moderately to severely active UC and CD). The invention is based, at least in part, on the discovery that immunotherapies including an anti-TL1A antibody can be useful in the treatment of IBD. Compositions, uses, and kits involving such antibodies are also provided herein.

UC is a chronic gastrointestinal inflammatory disorder that is characterized by diffuse mucosal inflammation involving the rectum with continuous extension into the colon. Although there are many risk factors associated with the development of UC, the disease fundamentally represents dysregulation of the mucosal immune system among genetically susceptible individuals in response to commensal microbiota and other environmental triggers.

The burden of UC is rising, with worldwide incidence and prevalence increasing over time (Ungaro et al., Lancet, 389:1756-1770, 2017; Kaplan et al., Nat Rev Gastroenterol Hepatol, 18:56-66, 2021; Lewis et al., Gastroenterology, 165:1197-1205, 2023). The disease can affect people of any age, but the peak age of onset is between 15 and 30 years, with a second peak between 50 and 70 years (Ordás et al., Lancet, 380:1606-1619, 2012).

According to the recent STRIDE-II guidelines, the short-term goal rated as most important by patients is symptomatic relief. Long-term treatment targets include clinical remission, endoscopic healing, restoration of quality of life, and absence of disability (Turner et al., Gastroenterology, 160:1570-1583, 2021).

Medical therapies, including inhibition of pro-inflammatory cytokines and adhesion molecules, have been shown to deliver therapeutic benefits, although a ceiling remains, with UC remission rates of approximately 20%-30% in induction trials (Alsoud et al., Lancet Gastroenterol Hepatol, 6:589-595, 2021).

Recent estimates of remission rates from an international survey are 37%-55% with current treatments; however, 22%-29% of patients have experienced a loss of response to current medications (i.e., anti-TNF therapy, anti-integrin, JAK inhibitor, or anti-IL-12/23), highlighting the need for more durable treatment options for patients with UC (Rubin et al., Inflamm Bowel Dis, 27:1942-1953, 2021).

Available advanced therapies are associated with various risks or adverse drug reactions, such as serious infections, cardiovascular events, thrombosis, and malignancies (Bhat et al., Inflamm Bowel Dis, 30 (5): 829-843, 2023). These safety considerations must be balanced with patient-specific factors and such considerations may eliminate some treatment options for individual patients.

Colectomy is required in up to 20%-30% of patients with UC who have uncontrolled ongoing inflammation that is refractory to medical therapies. While this is an appropriate therapeutic strategy for patients, these colorectal surgical procedures are also associated with early and late complications (Peyrin-Biroulet et al., Aliment Pharmacol Ther, 44:807-816, 2016; Fradet et al., Int J Surg Open, 22:22-32, 2020). These data show that there is a need for more robust and well-tolerated therapies with durable efficacy for patients with UC who have significantly impacted quality of life.

Crohn's disease (CD) is a chronic, progressive inflammatory disease of the gastrointestinal tract, characterized by periods of relapse and remission, which can ultimately lead to bowel damage and disability. Most patients present with an inflammatory phenotype, but over time, uncontrolled inflammation can lead to complications such as fibrotic strictures, fistula formation, or intestinal neoplasia. Half of all patients with CD develop intestinal complications within 20 years of diagnosis leading to surgical intervention.

The current goals of CD treatment are to induce and maintain clinical and endoscopic remission, halt the progressive course of disease, and prevent long-term complications. Current available therapies such as corticosteroids, immunosuppressants, and advanced therapies, including biological agents, are effective in many patients; however, the long-term efficacy rates remain unsatisfactory, with up to 30% of patients not exhibiting an initial response to treatment, and up to 50% of patients losing response over time. Furthermore, currently available advanced therapies are associated with various risks or adverse drug reactions such as serious infections, cardiovascular events, thrombosis, and malignancies.

Despite available treatments, disease progression and complications result in an estimated 50%-80% of CD patients requiring surgery in their lifetime. While timely surgery is appropriate to avoid complications, surgery is not curative and carries risks of postoperative complications with associated risks of mortality. Furthermore, postoperative recurrence is common, depending on patient risk factors, such that approximately 50% of patients have endoscopic recurrence within one year of ileocolic resection. Repeated surgical procedures can result in short bowel syndrome with chronic malabsorption and potential dependence on total parenteral nutrition. Therefore, an unmet need exists for more safe and effective treatments for CD.

### I. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.”

As used herein, the term “induction phase” refers to a series of one or more doses or dosing cycles (e.g., about 2-6 doses or dosing cycles) of one or more therapeutic agents (e.g., an anti-TL1A antibody (e.g., afimkibart) administered to a subject, wherein the one or more doses or dosing cycles are optionally followed by a maintenance phase.

The term “maintenance phase” as used herein refers to a series of one or more doses or dosing cycles of one or more therapeutic agents (e.g., an anti-TL1A antibody (e.g., afimkibart) that are administered to a subject subsequent to an induction phase with no relevant intervening surgery (i.e., no intervening surgery relating to the disease or condition intended to be treated by the one or more therapeutic agents). In some instances, the maintenance phase is initiated only if the subject did not experience disease progression or unacceptable toxicity during the induction phase. The induction phase and maintenance phase may or may not comprise use of the same therapeutic agents.

As used herein, “afimkibart” (also known as RO7790121, RVT-3101, or PF-06480605) is an antibody that binds tumor necrosis factor (TNF) superfamily protein TNF-like 1A (TL1A) and comprises the heavy chain sequence of SEQ ID NO: 9 and the light chain sequence of SEQ ID NO: 10.

The terms “antibody that binds to TL1A” and “anti-TL1A antibody” refer to an antibody that is capable of binding TL1A with sufficient affinity such that the antibody is useful as a diagnostic and/or therapeutic agent in targeting TL1A. In one aspect, the extent of binding of an anti-TL1A antibody to an unrelated, non-TL1A protein is less than about 10% of the binding of the antibody to TL1A as measured, e.g., by surface plasmon resonance (SPR). In one aspect, an antibody that binds to TL1A has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). The term “antibody” encompasses various antibody structures exhibiting the desired antigen-binding activity, including but not limited to: monoclonal antibodies, multispecific antibodies (e.g., bispecific antibodies) and antibody fragments.

The term “monoclonal antibody” as used herein refers to an antibody obtained from a population of substantially homogeneous antibodies, i.e. the antibodies forming this population are essentially identical, except for possible post-translational modifications arising e.g. during manufacturing and/or storage. These antibodies are directed against the same epitope (or the same group of epitopes in the case of multispecific monoclonal antibodies, e.g. the same pair of epitopes in the case of bispecific monoclonal antibodies). This definition expressly excludes polyclonal antibody preparations which are mixtures of antibodies directed against different epitopes. Monoclonal antibodies in accordance with the present invention may be made by a variety of techniques, including but not limited to hybridoma methodology, recombinant DNA methods, phage-display methods, and methods utilizing transgenic animals containing all or part of the human immunoglobulin loci, such methods and other exemplary methods for making monoclonal antibodies being described herein.

The term “full-length antibody” refers to an antibody having the structure of an immunoglobulin comprising two light chains and two heavy chains, and comprising an Fc region as defined herein. In one aspect, the antibody is a full-length IgG1 antibody.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to an antibody comprising amino acid residues from non-human CDRs and amino acid residues from human FRs. In one aspect, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Native antibodies” refer to naturally occurring immunoglobulin molecules with varying structures. For example, native IgG antibodies are heterotetrameric glycoproteins of about 150,000 daltons, composed of two identical light chains (LC) and two identical heavy chains (HC) that are disulfide-bonded. From N- to C-terminus, each heavy chain has a heavy chain variable domain (VH), also called a variable heavy domain or a heavy chain variable region, followed by three heavy chain constant domains (CH1, CH2, and CH3). Similarly, from N- to C-terminus, each light chain has a light chain variable domain (VL), also called a variable light domain or a light chain variable region, followed by a light chain constant domain (CL).

An “antibody fragment” refers to a molecule other than a full-length antibody that comprises a portion of a full-length antibody that binds the antigen to which the full-length antibody binds. Examples of antibody fragments include but are not limited to Fv molecules, Fab molecules, Fab′ molecules, Fab′-SH molecules, F (ab′) 2 molecules, diabodies, linear antibody molecules, single-chain antibody molecules (e.g., scFv and scFab molecules), and multispecific (e.g. bispecific) antibodies formed from antibody fragments.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Glutamine or glutamate residues at the N-terminus of antibody heavy or light chains may be converted to pyro-glutamate spontaneously (see e.g. Liu et al., Journal of Pharmaceutical Sciences 97, 2426-2447 (2008), Rehder et al., Journal of Chromatography A 1102, 164-175 (2006), Chelius et al., Anal Chem 78, 2370-2376 (2006)). Hence, variable domains disclosed herein which comprise either a glutamine (Q) or a glutamate (E) amino acid residue at the N-terminus of the antibody heavy or light chain, may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N-terminal pyro-glutamate (pyroE) residue instead of the N-terminal Q or E residue. Accordingly, for each antibody heavy chain, light chain, or variable domain sequence disclosed herein that contains an N-terminal Q or E residue, the corresponding sequence with an N-terminal pyroE residue is also encompassed.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat, E. A., et al., Sequences of Proteins of Immunological Interest, 5th ed., Public Health Service, National Institutes of Health, Bethesda, MD (1991) NIH Publication 91-3242 (hereinafter “Kabat 1991”). In one aspect, for the VL, the subgroup is subgroup kappa I as in Kabat 1991. In one aspect, for the VH, the subgroup is subgroup III as in Kabat 1991.

The term “complementarity determining region” or “CDR” as used herein refers to each of the regions of an antibody variable domain which are hypervariable in sequence and which determine antigen binding specificity. Generally, antibodies comprise six CDRs: three in the VH (CDR-H1, CDR-H2, CDR-H3), and three in the VL (CDR-L1, CDR-L2, CDR-L3). CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. Software programs (e.g., abYsis: http://www.abysis.org/abysis/sequence_input/key_annotation/key_annotation.cgi) are available and known to those of skill in the art for analysis of antibody sequences and determination of CDRs.

Exemplary CDRs herein include (numbering of amino acid residues according to the reference cited, i.e. Chothia numbering for the Chothia and Contact definition, Kabat numbering for the Kabat definition and IMGT numbering for the IMGT definition):
(a) hypervariable loops occurring at amino acid residues 26-32 (L1), 50-52 (L2), 91-96 (L3), 26-32 (H1), 53-55 (H2), and 96-101 (H3), according to Chothia and Lesk, J. Mol. Biol. 196:901-917 (1987) (“Chothia definition”);
(b) CDRs occurring at amino acid residues 24-34 (L1), 50-56 (L2), 89-97 (L3), 31-35B (H1), 50-65 (H2), and 95-102 (H3), according to Kabat 1991 (“Kabat definition”);
(c) antigen contacts occurring at amino acid residues 30-36 (L1), 46-55 (L2), 89-96 (L3), 30-35 (H1), 47-58 (H2), and 93-101 (H3), according to MacCallum et al. J. Mol. Biol. 262:732-745 (1996) (“Contact definition”); and
(d) CDRs occurring at amino acid residues residues 27-38 (L1), 56-65 (L2), 105-117 (L3), 27-38 (H1), 56-65 (H2), and 105-117 (H3), according to Lefranc et al. Dev. Comp. Immunol. 27:55-77 (2003) (“IMGT definition”).

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used herein denotes a constant region of a human antibody, in particular a heavy chain constant region of a human antibody of the subclass IgG1, IgG2, IgG3, or IgG4 and/or a light chain kappa or lambda constant region. Such constant regions are well known in the state of the art and e.g. described by Kabat 1991. Unless otherwise specified herein, numbering of amino acid residues in the constant region is according to the numbering system as described in Kabat 1991. Specifically, the Kabat numbering system (referred to as “numbering according to Kabat” or “Kabat numbering” herein; see pages 647-660 of Kabat 1991) is used for the light chain constant domain of kappa and lambda isotype, and the Kabat EU index numbering system (referred to as “numbering according to Kabat EU index” or “Kabat EU index numbering” herein, see pages 661-723 of Kabat 1991) is used for the heavy chain constant domains.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case in particular where the final two C-terminal amino acids of the heavy chain are glycine (G446) and lysine (K447, Kabat EU numbering). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal lysine if not indicated otherwise. The corresponding sequence including a C-terminal lysine residue is also encompassed, however. Accordingly, in one aspect, a heavy chain including an Fc region as specified herein comprises an additional C-terminal lysine residue (K447, Kabat EU numbering). Also encompassed is the corresponding sequence without the C-terminal glycine residue. Accordingly, in one aspect, a heavy chain including an Fc region as specified herein lacks the C-terminal glycine residue (G446, Kabat EU numbering). In such a heavy chain, the C-terminal amino acid residue may be proline (P445, Kabat EU numbering) or proline amide (P445-NH2, Kabat EU numbering). Unless otherwise specified herein, numbering of amino acid residues in the Fc region or heavy chain constant region is according to the EU numbering system, also called the EU index, as described in Kabat 1991.

“Effector functions” refer to those biological activities attributable to the Fc region of an antibody, which vary with the antibody isotype. Examples of antibody effector functions include: C1q binding, complement dependent cytotoxicity (CDC), Fc receptor binding, antibody-dependent cell-mediated cytotoxicity (ADCC), antibody-dependent cellular phagocytosis (ADCP), down regulation of cell surface receptors (e.g., B cell receptor), and B cell activation.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. A preferred method for measuring affinity is Surface Plasmon Resonance (SPR).

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC, affinity chromatography, size exclusion chromatography) methods. For a review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Suitable host cells may include, for example, CHO cells, HEK-293 cells, Expi293F cells, PER.C6 cells, NSO cells, lymphocytic cells, prokaryotic cells such as E. coli, and other eukaryotic hosts such as plant cells and fungi. Human host cells are included with the proviso that they are not used within the human body.

A “naked antibody” refers to an antibody that is not conjugated to a heterologous moiety (e.g., a cytotoxic moiety) or radiolabel. The naked antibody may be present in a pharmaceutical composition.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, MegAlign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis” Proc. Nat. Acad. Sci. 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

An “immunoconjugate” is an antibody conjugated to one or more heterologous molecule(s), including but not limited to one or more cytotoxic agent(s).

As used herein, the term “treatment” refers to clinical intervention in an attempt to alter the natural course of a disease during the course of clinical pathology. Desirable effects of treatment include decreasing the rate of disease progression, ameliorating or palliating the disease state, and remission or improved prognosis. For purposes of this disclosure, beneficial or desired clinical results include reduction or improvement in signs and symptoms of inflammatory bowel disease (IBD) (e.g., ulcerative colitis (UC) or Crohn's disease (CD)), for example as compared to before administration of the anti-TL1A antibody.

An “effective amount” of an agent, e.g., a pharmaceutical composition, refers to an amount of the antibody or medicament effective, at dosages and for periods of time necessary, to achieve the desired treatment as defined above. In more specific aspects, an effective amount prevents, alleviates or ameliorates signs or symptoms of IBD, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more signs or symptoms of IBD, decreasing the dose of other medications required to treat the disease, enhancing the effect of another medication, and/or delaying the progression of the disease in patients. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

Herein, an “effective amount” may refer to the amount of a therapeutic agent (e.g., an anti-TL1A antibody (e.g., afimkibart) or a combination of therapeutic agents (e.g., an anti-TL1A antibody and one or more additional therapeutic agents)), that achieves a therapeutic result. In some examples, the effective amount of a therapeutic agent or a combination of therapeutic agents is the amount of the agent or of the combination of agents that achieves a clinical endpoint of clinical remission, clinical response (e.g., improved modified Mayo score (mMS) or partial modified Mayo score (pmMS)), endoscopic improvement, endoscopic remission, histologic-endoscopic mucosal improvement, histologic-endoscopic remission, and/or corticosteroid-free remission. Improvement (e.g., in terms of clinical remission) may be relative to a suitable reference treatment, for example, treatment that does not include the anti-TL1A antibody.

“Ameliorating” means a lessening or improvement of one or more signs or symptoms of IBD (e.g., UC or CD), for example as compared to not administering an anti-TL1A antibody as described herein. “Ameliorating” also includes shortening or reduction in duration of a symptom.

The term “preventing” or “prevent” refers to (a) keeping a disorder from occurring or (b) delaying the onset of a disorder or onset of symptoms of a disorder.

Treatment “effectively improves” or “effectively reduces” when assessment of the sign or symptom of IBD is quantified via a clinical measure relative to baseline and during and/or after the treatment period. The difference between the clinical measure at baseline and during/after treatment is compared and used to determine whether the sign or symptom has improved and the treatment is effective. This comparison can include comparison to placebo or to one or more of the prior therapies.

A “patient,” an “individual,” or a “subject,” used interchangeably herein, is a mammal. In one aspect, the individual or subject is a human. In one aspect, the individual is in need of treatment with the medicament or antibody disclosed herein.

The term “pharmaceutical composition” or “pharmaceutical formulation” refers to a preparation of the antibody and one or more pharmaceutically acceptable carriers or excipients.

A “pharmaceutically acceptable carrier” refers to an ingredient in a pharmaceutical composition or formulation, other than an active ingredient, which is nontoxic to a subject and includes, but is not limited to, a buffer, excipient, stabilizer, surfactant, and/or preservative.

The term “subcutaneous administration” refers to the administration of a substance into the subcutaneous layer.

II. Methods of Treating Inflammatory Bowel Disease with Anti-TL1A Antibodies

In some aspects, the disclosure provides a method of treating an inflammatory bowel disease (IBD) (e.g., ulcerative colitis (UC) or Crohn's disease (CD)) in a patient, the method comprising administering to the patient an effective amount of an anti-TNF-like ligand 1A (TL1A) antibody (e.g., an anti-TL1A antibody provided in Section III herein) in a dosing regimen comprising at least a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose.

In some aspects, the disclosure provides a method of treating an IBD (e.g., UC or CD) in a patient, the method comprising administering to the patient an effective amount of an anti-TL1A antibody (e.g., an anti-TL1A antibody provided in Section III herein) in a dosing regimen comprising a first phase (e.g., an induction phase) and a second phase (e.g., a maintenance phase), wherein: (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks. In some aspects, the anti-TL1A antibody comprises the following complementarity-determining regions (CDRs): (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

The disclosure also provides an anti-TL1A antibody for use in treating IBD in a patient according to the methods provided herein and use of an anti-TL1A antibody in the manufacture of a medicament for treating IBD according to the methods provided herein.

As a general principle, the treatment of IBD with anti-TL1A antibodies is described in PCT Publication No. WO 2021/260577 and in U.S. Patent Application Publication No. US 2023/0235070 A1, which are incorporated herein by reference for all purposes.

The methods of treatment and related uses provided herein may include one, two, or all three of a first phase (e.g., an induction phase), a second phase (e.g., a maintenance phase), and an extension phase.

### A. First Phase

Any of the methods for treating IBD provided herein may comprise administration of an anti-TL1A antibody in at least a first phase (e.g., an induction phase). The induction phase may comprise administration of one or more doses of the anti-TL1A antibody (e.g., may comprise administration of one, two, three, or four doses of the anti-TL1A antibody).

In some aspects, the first phase (e.g., induction phase) comprises administration of four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose. In some aspects, the first phase (e.g., induction phase) has a duration of about 12 weeks, and (a) the first dose of the anti-TL1A antibody is administered on about Day 1 of Week 0 (e.g., Day 1 ±3 days); (b) the second dose of the anti-TL1A antibody is administered on about Day 1 of Week 2 (e.g., Day 1 ±3 days); (c) the third dose of the anti-TL1A antibody is administered on about Day 1 of Week 6 (e.g., Day 1 ±3 days); and (d) the fourth dose of the anti-TL1A antibody is administered on about Day 1 of Week 10 (e.g., Day 1 ±3 days). In some aspects, the first phase (e.g., induction phase) has a duration of about 12 weeks, and (a) the first dose of the anti-TL1A antibody is administered on Day 1 of Week 0; (b) the second dose of the anti-TL1A antibody is administered on Day 1 of Week 2; (c) the third dose of the anti-TL1A antibody is administered on Day 1 of Week 6; and/or (d) the fourth dose of the anti-TL1A antibody is administered on Day 1 of Week 10 (e.g., first phase (e.g., induction phase) doses according to one, two, three, or all four of (a), (b), (c), and (d) are administered). In some aspects, the first phase (e.g., induction phase) has a duration of about 12 weeks, and (a) the first dose of the anti-TL1A antibody is administered on Day 1 of Week 0; (b) the second dose of the anti-TL1A antibody is administered on Day 1 of Week 2; (c) the third dose of the anti-TL1A antibody is administered on Day 1 of Week 6; and (d) the fourth dose of the anti-TL1A antibody is administered on Day 1 of Week 10.

The anti-TL1A antibody may be administered at a dose of, e.g., 1, 5, 10, 25, 50, 100, 150, 200, 250, 300, 350, 400, 450, 500, 550, 600, 650, 700, 750, 800, 850, 900, 950 or 1000 mg in the first phase (e.g., induction phase). In some embodiments, each dose of the anti-TL1A antibody in the first phase (e.g., induction phase) is about 50 mg to about 600 mg, for example, about 50 mg to about 150 mg, about 150 mg to about 450 mg, about 150 mg to about 500 mg, or about 450 mg to about 600 mg. In some embodiments, each dose of the anti-TL1A antibody in the first phase (e.g., induction phase) is about 50 mg, about 150 mg, about 450 mg, about 500 mg or about 600 mg. In some aspects, the anti-TL1A antibody is administered at a dose of 50, 150, or 450 mg during the first phase (e.g., induction phase). In one aspect, the anti-TL1A antibody is administered at a dose of about 500 mg (e.g., at a dose of 500 mg) during the first phase (e.g., induction phase). The first phase (e.g., induction phase) doses can be administered by any means. Preferably, the first phase (e.g., induction phase) doses are administered intravenously or subcutaneously. In some aspects, in the first phase (e.g., induction phase), the anti-TL1A antibody is administered intravenously (IV) at a dose of about 500 mg (e.g., a dose of 500 mg) (e.g., each dose in the first phase (e.g., induction phase) comprises IV administration of about 500 mg (e.g., 500 mg) of the anti-TL1A antibody).

In some aspects, in the first phase (e.g., induction phase), the anti-TL1A antibody is administered subcutaneously (SC) at a dose of about 500 mg (e.g., a dose of 500 mg) (e.g., each dose in the first phase (e.g., induction phase) comprises SC administration of about 500 mg (e.g., 500 mg) of the anti-TL1A antibody).

Accordingly, in some aspects, the disclosure provides a method of treating an IBD (e.g., UC or CD) in a patient, the method comprising administering to the patient an effective amount of an anti-TL1A antibody (e.g., an anti-TL1A antibody provided in Section III herein) in a dosing regimen comprising a first phase (e.g., an induction phase), wherein the first phase (e.g., induction phase) comprises intravenous administration of only four doses of the anti-TL1A antibody at a dose of about 500 mg, wherein the first phase (e.g., induction phase) has a duration of about 12 weeks, and (a) the first dose of the anti-TL1A antibody is administered on Day 1 of Week 0; (b) the second dose of the anti-TL1A antibody is administered on Day 1 of Week 2; (c) the third dose of the anti-TL1A antibody is administered on Day 1 of Week 6; and (d) the fourth dose of the anti-TL1A antibody is administered on Day 1 of Week 10. In some aspects, the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating an IBD (e.g., UC or CD) in a patient, the method comprising administering to the patient an effective amount of an anti-TL1A antibody (e.g., an anti-TL1A antibody provided in Section III herein) in a dosing regimen comprising intravenous (IV) administration of only four doses of the anti-TL1A antibody at a dose of about 500 mg, wherein the four doses of the anti-TL1A antibody are administered over about 12 weeks, and (a) the IV first dose of the anti-TL1A antibody is administered on Day 1 of Week 0; (b) the second IV dose of the anti-TL1A antibody is administered on Day 1 of Week 2; (c) the third IV dose of the anti-TL1A antibody is administered on Day 1 of Week 6; and (d) the fourth IV dose of the anti-TL1A antibody is administered on Day 1 of Week 10. In some aspects, the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating IBD (e.g., UC or CD) in a patient, the method comprising administering to the patient an effective amount of an anti-TL1A antibody in a dosing regimen comprising a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises (a) a heavy chain variable (VH) domain comprising an amino acid sequence having at least 95% sequence identity to the amino acid sequence of SEQ ID NO: 1; and/or (b) a light chain variable (VL) domain comprising an amino acid sequence having at least 95% sequence identity to the amino acid sequence of SEQ ID NO: 2.

In some aspects, the disclosure provides a method of treating IBD (e.g., UC or CD) in a patient, the method comprising administering to the patient an effective amount of an anti-TL1A antibody in a dosing regimen comprising a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises (I) a heavy chain variable (VH) domain comprising an amino acid sequence having at least 95% sequence identity to the amino acid sequence of SEQ ID NO: 1 and/or (II) a light chain variable (VL) domain comprising an amino acid sequence having at least 95% sequence identity to the amino acid sequence of SEQ ID NO: 2, and comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects, the disclosure provides a method of treating IBD (e.g., UC or CD) in a patient, the method comprising administering to the patient an effective amount of an anti-TL1A antibody in a dosing regimen comprising administration of only four IV doses of the anti-TL1A antibody, wherein: (i) the second IV dose of the anti-TL1A antibody is administered about two weeks after the first dose; (ii) the third IV dose of the anti-TL1A antibody is administered about four weeks after the second dose; and (iii) the fourth IV dose of the anti-TL1A antibody is administered about four weeks after the third dose; wherein the anti-TL1A antibody comprises (a) a VH domain comprising an amino acid sequence having at least 95% sequence identity to the amino acid sequence of SEQ ID NO: 1; and/or (b) a VL domain comprising an amino acid sequence having at least 95% sequence identity to the amino acid sequence of SEQ ID NO: 2.

In some aspects, the disclosure provides a method of treating IBD (e.g., UC or CD) in a patient, the method comprising administering to the patient an effective amount of an anti-TL1A antibody in a dosing regimen comprising administration of only four IV doses of the anti-TL1A antibody, wherein: (i) the second IV dose of the anti-TL1A antibody is administered about two weeks after the first dose; (ii) the third IV dose of the anti-TL1A antibody is administered about four weeks after the second dose; and (iii) the fourth IV dose of the anti-TL1A antibody is administered about four weeks after the third dose; wherein the anti-TL1A antibody comprises (I) a heavy chain variable (VH) domain comprising an amino acid sequence having at least 95% sequence identity to the amino acid sequence of SEQ ID NO: 1 and/or (II) a light chain variable (VL) domain comprising an amino acid sequence having at least 95% sequence identity to the amino acid sequence of SEQ ID NO: 2, and comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects, the disclosure provides a method of treating IBD (e.g., UC or CD) in a patient, the method comprising administering to the patient an effective amount of an anti-TL1A antibody in a dosing regimen comprising a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises (a) a VH domain comprising the amino acid sequence of SEQ ID NO: 1; and/or (b) a VL domain comprising the amino acid sequence of SEQ ID NO: 2.

In some aspects, the disclosure provides a method of treating IBD (e.g., UC or CD) in a patient, the method comprising administering to the patient an effective amount of an anti-TL1A antibody in a dosing regimen comprising administration of only four IV doses of the anti-TL1A antibody, wherein: (i) the second IV dose of the anti-TL1A antibody is administered about two weeks after the first dose; (ii) the third IV dose of the anti-TL1A antibody is administered about four weeks after the second dose; and (iii) the fourth IV dose of the anti-TL1A antibody is administered about four weeks after the third dose; wherein the anti-TL1A antibody comprises (a) a VH domain comprising the amino acid sequence of SEQ ID NO: 1; and/or (b) a VL domain comprising the amino acid sequence of SEQ ID NO: 2.

In some aspects, the disclosure provides a method of treating IBD (e.g., UC or CD) in a patient, the method comprising administering to the patient an effective amount of an anti-TL1A antibody in a dosing regimen comprising a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises (a) a heavy chain comprising an amino acid sequence having at least 95% sequence identity to SEQ ID NO: 9; and (b) a light chain comprising an amino acid sequence having at least 95% sequence identity to SEQ ID NO: 10. In some aspects, the anti-TL1A antibody is afimkibart.

In some aspects, the disclosure provides a method of treating IBD (e.g., UC or CD) in a patient, the method comprising administering to the patient an effective amount of an anti-TL1A antibody in a dosing regimen comprising a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises (I) a heavy chain comprising an amino acid sequence having at least 95% sequence identity to SEQ ID NO: 9; and (II) a light chain comprising an amino acid sequence having at least 95% sequence identity to SEQ ID NO: 10, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating IBD (e.g., UC or CD) in a patient, the method comprising administering to the patient an effective amount of an anti-TL1A antibody in a dosing regimen comprising administration of only four IV doses of the anti-TL1A antibody, wherein: (i) the second IV dose of the anti-TL1A antibody is administered about two weeks after the first dose; (ii) the third IV dose of the anti-TL1A antibody is administered about four weeks after the second dose; and (iii) the fourth IV dose of the anti-TL1A antibody is administered about four weeks after the third dose; wherein the anti-TL1A antibody comprises (a) a heavy chain comprising an amino acid sequence having at least 95% sequence identity to SEQ ID NO: 9; and (b) a light chain comprising an amino acid sequence having at least 95% sequence identity to SEQ ID NO: 10. In some aspects, the anti-TL1A antibody is afimkibart.

In some aspects, the disclosure provides a method of treating IBD (e.g., UC or CD) in a patient, the method comprising administering to the patient an effective amount of an anti-TL1A antibody in a dosing regimen comprising administration of only four IV doses of the anti-TL1A antibody, wherein: (i) the second IV dose of the anti-TL1A antibody is administered about two weeks after the first dose; (ii) the third IV dose of the anti-TL1A antibody is administered about four weeks after the second dose; and (iii) the fourth IV dose of the anti-TL1A antibody is administered about four weeks after the third dose; wherein the anti-TL1A antibody comprises (I) a heavy chain comprising an amino acid sequence having at least 95% sequence identity to SEQ ID NO: 9; and (II) a light chain comprising an amino acid sequence having at least 95% sequence identity to SEQ ID NO: 10, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart. In some aspects, the disclosure provides a method of treating IBD (e.g., UC or CD) in a patient, the method comprising administering to the patient an effective amount of an anti-TL1A antibody in a dosing regimen comprising a first phase (e.g., an induction phase), wherein the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; wherein the anti-TL1A antibody comprises (a) a heavy chain comprising the amino acid sequence of SEQ ID NO: 9; and (b) a light chain comprising the amino acid sequence of SEQ ID NO: 10.

In some aspects, the disclosure provides a method of treating IBD (e.g., UC or CD) in a patient, the method comprising administering to the patient an effective amount of an anti-TL1A antibody in a dosing regimen comprising administration of only four IV doses of the anti-TL1A antibody, wherein: (i) the second IV dose of the anti-TL1A antibody is administered about two weeks after the first dose; (ii) the third IV dose of the anti-TL1A antibody is administered about four weeks after the second dose; and (iii) the fourth IV dose of the anti-TL1A antibody is administered about four weeks after the third dose; wherein the anti-TL1A antibody comprises (a) a heavy chain comprising the amino acid sequence of SEQ ID NO: 9; and (b) a light chain comprising the amino acid sequence of SEQ ID NO: 10.

### B. Second Phase

Any of the methods provided herein may comprise administration of an anti-TL1A antibody in a second phase (e.g., a maintenance phase) that follows administration of the anti-TL1A antibody in a first phase (e.g., an induction phase). The second phase (e.g., maintenance phase) may comprise administration of one or more doses of the anti-TL1A antibody.

The second phase (e.g., maintenance phase) may be initiated at any appropriate time following the first phase (e.g., induction phase). In some aspects, the first dose of the second phase (e.g., maintenance phase) is administered about two weeks (e.g., two weeks) after administration of the last dose of the first phase (e.g., induction phase). For example, in aspects in which the first phase (e.g., induction phase) comprises administration of four doses of the anti-TL1A antibody, the first dose of the second phase (e.g., maintenance phase) may be administered about two weeks (e.g., two weeks) after administration of the fourth dose of the first phase (e.g., induction phase).

In some aspects in which the second phase (e.g., maintenance phase) comprises administration of at least two doses of the anti-TL1A antibody, the time interval between each individual dose in the second phase (e.g., maintenance dose) may be the same. In other aspects, the time interval between individual doses in the second phase (e.g., maintenance phase doses) is not the same. The individual doses in the second phase (e.g., maintenance phase) can be administered at least daily, at least one day apart, at least 1 week apart, at least 2 weeks apart, at least 3 weeks apart, at least 1 month apart, at least 2 months apart, at least 3 months apart, at least 4 months apart, at least 5 months apart, or at least 6 months apart. In one preferred aspect, the individual doses in the second phase (e.g., maintenance doses) are administered one month apart.

In some aspects, the second phase (e.g., maintenance phase) comprises administration (e.g., subcutaneous administration) of the anti-TL1A antibody every four weeks (Q4W).

In some aspects, the second phase (e.g., maintenance phase) comprises administration (e.g., subcutaneous administration) of the anti-TL1A antibody every two weeks (Q2W).

In some aspects, the second phase (e.g., maintenance phase) comprises (i) at least one interval in which the anti-TL1A antibody is administered every four weeks (Q4W) and (ii) at least one interval in which the anti-TL1A antibody is administered every two weeks (Q2W). In some aspects, the second phase (e.g., maintenance phase) dosing frequency of a subject is increased from Q4W to Q2W based on observation of disease worsening.

The anti-TL1A antibody may be administered at a dose of, e.g., 1, 5, 10, 25, 50, 100, 150, 200, 250, 300, 350, 400, 450, 500, 550, 600, 650, 700, 750, 800, 850, 900, 950 or 1000 mg during the second (e.g., maintenance) phase. In some aspects, the anti-TL1A antibody is administered at a dose of 50, 150 or 450 mg during the second (e.g., maintenance) phase. In one aspect, the anti-TL1A antibody is administered at a dose of about 150 mg (e.g., at a dose of 150 mg) during the second (e.g., maintenance) phase. In one aspect, the anti-TL1A antibody is administered at a dose of about 450 mg (e.g., at a dose of 450 mg) during the second (e.g., maintenance) phase. Doses in the second (e.g., maintenance) phase can be administered by any means (e.g., may be administered intravenously or subcutaneously). For example, in some aspects, in the second (e.g., maintenance) phase, the anti-TL1A antibody is administered subcutaneously at a dose of 50, 150 or 450 mg. In some aspects, the anti-TL1A antibody is administered subcutaneously at a dose of 50, 150 or 450 mg every four weeks (Q4W).

In some aspects, in the second (e.g., maintenance) phase, the anti-TL1A antibody is administered subcutaneously at a dose of about 150 mg (e.g., 150 mg) every four weeks. In some aspects, in the second (e.g., maintenance) phase, the anti-TL1A antibody is administered subcutaneously at a dose of about 150 mg (e.g., 150 mg) every two weeks. In some aspects, each dose in the second (e.g., maintenance) phase is about 150 mg (e.g., 150 mg), and the second (e.g., maintenance) phase comprises (i) at least one interval in which the anti-TL1A antibody is subcutaneously administered every four weeks (Q4W) and (ii) at least one interval in which the anti-TL1A antibody is subcutaneously administered every two weeks (Q2W).

In some aspects, in the second (e.g., maintenance) phase, the anti-TL1A antibody is administered subcutaneously at a dose of about 450 mg (e.g., 450 mg) every four weeks. In some aspects, in the second (e.g., maintenance) phase, the anti-TL1A antibody is administered subcutaneously at a dose of about 450 mg (e.g., 450 mg) every two weeks. In some aspects, each dose in the second (e.g., maintenance) phase is about 450 mg (e.g., 450 mg), and the second (e.g., maintenance) phase comprises (i) at least one interval in which the anti-TL1A antibody is subcutaneously administered every four weeks (Q4W) and (ii) at least one interval in which the anti-TL1A antibody is subcutaneously administered every two weeks (Q2W).

In some aspects, the second phase (e.g., maintenance phase) comprises administration of at least 2, 3, 4, 5, 6, 7, 8, 9, 10, 11, 12, 13, 14, 15, 16, 17, 18, 19, 20, 21, 22, 23, 24, 25, or more than 25 doses of the anti-TL1A antibody.

In some aspects, the second phase (e.g., maintenance phase) comprises eleven doses of the anti-TL1A antibody (e.g., comprises eleven doses of the anti-TL1A antibody administered in a Q4W dosing regimen). In some aspects, the second phase (e.g., maintenance phase) has a duration of about 40 weeks.

Accordingly, in some aspects, the disclosure provides a method of treating an IBD (e.g., UC or CD) in a patient, the method comprising administering to the patient an effective amount of an anti-TL1A antibody (e.g., an anti-TL1A antibody provided in Section III herein) in a dosing regimen comprising a first phase and a second phase (e.g., an induction phase and a maintenance phase), wherein: (a) the first phase (e.g., induction phase) comprises intravenous administration of four doses of the anti-TL1A antibody at a dose of about 500 mg, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises subcutaneous administration of the anti-TL1A antibody every four weeks at a dose of about 150 mg.

In some aspects, the disclosure provides a method of treating an IBD (e.g., UC or CD) in a patient, the method comprising administering to the patient an effective amount of an anti-TL1A antibody (e.g., an anti-TL1A antibody provided in Section III herein) in a dosing regimen comprising a first phase and a second phase (e.g., an induction phase and a maintenance phase), wherein: (a) the first phase (e.g., induction phase) comprises intravenous administration of four doses of the anti-TL1A antibody at a dose of about 500 mg, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises subcutaneous administration of the anti-TL1A antibody every four weeks at a dose of about 450 mg. In some aspects, the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating an IBD (e.g., UC or CD) in a patient, the method comprising administering to the patient an effective amount of an anti-TL1A antibody (e.g., an anti-TL1A antibody provided in Section III herein) in a dosing regimen comprising (a) intravenous administration of only four doses of the anti-TL1A antibody at a dose of about 500 mg, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third dose; and (b) subcutaneous administration of the anti-TL1A antibody every four weeks at a dose of about 450 mg. In some aspects, the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating an IBD (e.g., UC or CD) in a patient, the method comprising administering to the patient an effective amount of an anti-TL1A antibody in a dosing regimen comprising a first phase and a second phase (e.g., an induction phase and a maintenance phase), wherein (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks; wherein the anti-TL1A antibody comprises (a) a heavy chain variable (VH) domain comprising an amino acid sequence having at least 95% sequence identity to the amino acid sequence of SEQ ID NO: 1; and/or (b) a light chain variable (VL) domain comprising an amino acid sequence having at least 95% sequence identity to the amino acid sequence of SEQ ID NO: 2.

In some aspects, the disclosure provides a method of treating an IBD (e.g., UC or CD) in a patient, the method comprising administering to the patient an effective amount of an anti-TL1A antibody in a dosing regimen comprising a first phase and a second phase (e.g., an induction phase and a maintenance phase), wherein (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks; wherein the anti-TL1A antibody comprises (I) a heavy chain variable (VH) domain comprising an amino acid sequence having at least 95% sequence identity to the amino acid sequence of SEQ ID NO: 1 and/or (II) a light chain variable (VL) domain comprising an amino acid sequence having at least 95% sequence identity to the amino acid sequence of SEQ ID NO: 2, and comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects, the disclosure provides a method of treating an IBD (e.g., UC or CD) in a patient, the method comprising administering to the patient an effective amount of an anti-TL1A antibody in a dosing regimen comprising a first phase and a second phase (e.g., an induction phase and a maintenance phase), wherein (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks; wherein the anti-TL1A antibody comprises (a) a VH domain comprising the amino acid sequence of SEQ ID NO: 1; and/or (b) a VL domain comprising the amino acid sequence of SEQ ID NO: 2.

In some aspects, the disclosure provides a method of treating an IBD (e.g., UC or CD) in a patient, the method comprising administering to the patient an effective amount of an anti-TL1A antibody in a dosing regimen comprising a first phase and a second phase (e.g., an induction phase and a maintenance phase), wherein (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks; wherein the anti-TL1A antibody comprises: (a) a heavy chain comprising an amino acid sequence having at least 95% sequence identity to, or having the amino acid sequence of, SEQ ID NO: 9; and (b) a light chain comprising an amino acid sequence having at least 95% sequence identity to, or having the amino acid sequence of, SEQ ID NO: 10. In some aspects, the anti-TL1A antibody is afimkibart.

In some aspects, the disclosure provides a method of treating an IBD (e.g., UC or CD) in a patient, the method comprising administering to the patient an effective amount of an anti-TL1A antibody in a dosing regimen comprising a first phase and a second phase (e.g., an induction phase and a maintenance phase), wherein (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks; wherein the anti-TL1A antibody comprises (I) a heavy chain comprising an amino acid sequence having at least 95% sequence identity to SEQ ID NO: 9; and (II) a light chain comprising an amino acid sequence having at least 95% sequence identity to SEQ ID NO: 10, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating an IBD (e.g., UC or CD) in a patient, the method comprising administering to the patient an effective amount of an anti-TL1A antibody in a dosing regimen comprising a first phase and a second phase (e.g., an induction phase and a maintenance phase), wherein (a) the first phase (e.g., induction phase) comprises administration of only four doses of the anti-TL1A antibody, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises administration of the anti-TL1A antibody every four weeks; wherein the anti-TL1A antibody comprises: (a) a heavy chain comprising the amino acid sequence of SEQ ID NO: 9; and (b) a light chain comprising the amino acid sequence of SEQ ID NO: 10. In some aspects, the anti-TL1A antibody is afimkibart.

In some aspects, (a) the first dose of the first phase (e.g., first induction phase dose) of the anti-TL1A antibody is administered on about Day 1 of Week 0; (b) the second dose of the first phase (e.g., second induction phase dose) of the anti-TL1A antibody is administered on about Day 1 of Week 2; (c) the third dose of the first phase (e.g., third induction phase dose) of the anti-TL1A antibody is administered on about Day 1 of Week 6; (d) the fourth dose of the first phase (e.g., fourth induction phase dose) of the anti-TL1A antibody is administered on about Day 1 of Week 10; (e) the first dose of the second phase (e.g., first maintenance phase dose) is administered on about Day 1 of Week 12; and (f) the subsequent doses of the second phase (e.g., subsequent maintenance phase doses) are administered on about Day 1 of Weeks 16, 20, 24, 28, 32, 36, 40, 44, 48, and 52.

In some aspects, the disclosure provides a method of treating an IBD (e.g., UC or CD) in a patient, the method comprising administering to the patient an effective amount of an anti-TL1A antibody in a dosing regimen comprising (a) intravenous administration of only four doses of the anti-TL1A antibody, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third IV dose; and (b) subcutaneous (SC) administration of the anti-TL1A antibody every four weeks; wherein the anti-TL1A antibody comprises (a) a VH domain comprising an amino acid sequence having at least 95% sequence identity to the amino acid sequence of SEQ ID NO: 1; and/or (b) a VL domain comprising an amino acid sequence having at least 95% sequence identity to the amino acid sequence of SEQ ID NO: 2.

In some aspects, the disclosure provides a method of treating an IBD (e.g., UC or CD) in a patient, the method comprising administering to the patient an effective amount of an anti-TL1A antibody in a dosing regimen comprising (a) intravenous administration of only four doses of the anti-TL1A antibody, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third IV dose; and (b) subcutaneous (SC) administration of the anti-TL1A antibody every four weeks; wherein the anti-TL1A antibody comprises (I) a heavy chain variable (VH) domain comprising an amino acid sequence having at least 95% sequence identity to the amino acid sequence of SEQ ID NO: 1 and/or (II) a light chain variable (VL) domain comprising an amino acid sequence having at least 95% sequence identity to the amino acid sequence of SEQ ID NO: 2, and comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8.

In some aspects, the disclosure provides a method of treating an IBD (e.g., UC or CD) in a patient, the method comprising administering to the patient an effective amount of an anti-TL1A antibody in a dosing regimen comprising (a) intravenous administration of only four doses of the anti-TL1A antibody, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third IV dose; and (b) subcutaneous (SC) administration of the anti-TL1A antibody every four weeks; wherein the anti-TL1A antibody comprises (a) a VH domain comprising the amino acid sequence of SEQ ID NO: 1; and/or (b) a VL domain comprising the amino acid sequence of SEQ ID NO: 2.

In some aspects, the disclosure provides a method of treating an IBD (e.g., UC or CD) in a patient, the method comprising administering to the patient an effective amount of an anti-TL1A antibody in a dosing regimen comprising (a) intravenous administration of only four doses of the anti-TL1A antibody, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third IV dose; and (b) subcutaneous (SC) administration of the anti-TL1A antibody every four weeks; wherein the anti-TL1A antibody comprises: (a) a heavy chain comprising an amino acid sequence having at least 95% sequence identity to SEQ ID NO: 9; and (b) a light chain comprising an amino acid sequence having at least 95% sequence identity to SEQ ID NO: 10.

In some aspects, the disclosure provides a method of treating an IBD (e.g., UC or CD) in a patient, the method comprising administering to the patient an effective amount of an anti-TL1A antibody in a dosing regimen comprising (a) intravenous administration of only four doses of the anti-TL1A antibody, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third IV dose; and (b) subcutaneous (SC) administration of the anti-TL1A antibody every four weeks; wherein the anti-TL1A antibody comprises (I) a heavy chain comprising an amino acid sequence having at least 95% sequence identity to SEQ ID NO: 9; and (II) a light chain comprising an amino acid sequence having at least 95% sequence identity to SEQ ID NO: 10, and wherein the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

In some aspects, the disclosure provides a method of treating an IBD (e.g., UC or CD) in a patient, the method comprising administering to the patient an effective amount of an anti-TL1A antibody in a dosing regimen comprising (a) intravenous administration of only four doses of the anti-TL1A antibody, wherein: (i) the second IV dose of the anti-TL1A antibody is administered about two weeks after the first IV dose; (ii) the third IV dose of the anti-TL1A antibody is administered about four weeks after the second IV dose; and (iii) the fourth IV dose of the anti-TL1A antibody is administered about four weeks after the third IV dose; and (b) subcutaneous (SC) administration of the anti-TL1A antibody every four weeks; wherein the anti-TL1A antibody comprises: (a) a heavy chain comprising the amino acid sequence of SEQ ID NO: 9; and (b) a light chain comprising the amino acid sequence of SEQ ID NO: 10.

In some aspects, the anti-TL1A antibody is afimkibart. In some aspects, (a) the first IV dose of the anti-TL1A antibody is administered on about Day 1 of Week 0; (b) the second IV dose of the anti-TL1A antibody is administered on about Day 1 of Week 2; (c) the third IV dose of the anti-TL1A antibody is administered on about Day 1 of Week 6; (d) the fourth IV dose of the anti-TL1A antibody is administered on about Day 1 of Week 10; (e) the first SC dose of the anti-TL1A antibody is administered on about Day 1 of Week 12; and (f) the subsequent SC doses of the anti-TL1A antibody are administered on about Day 1 of Weeks 16, 20, 24, 28, 32, 36, 40, 44, 48, and 52. In some aspects, the dosing regimen comprising the first phase and second phase (e.g., induction phase and maintenance phase) has a duration of about 52 weeks (e.g., has a duration of 52 weeks).

### C. Extension Phase

Any of the methods provided herein may comprise administration of an anti-TL1A antibody in an extension phase that follows administration of the anti-TL1A antibody in (i) a first phase (e.g., an induction phase) or (ii) a first phase and a second phase (e.g., an induction phase and a maintenance phase). For example, the extension phase may follow (i) a first phase (e.g., induction phase) comprising only IV administration of the anti-TL1A antibody or (ii) a first phase (e.g., an induction phase) comprising only IV administration of the anti-TL1A antibody and a second phase (e.g., a maintenance phase) comprising SC administration of the anti-TL1A antibody. The extension phase may comprise administration of one or more doses of the anti-TL1A antibody.

The extension phase may be initiated at any appropriate time following the first phase (e.g., induction phase) or the second phase (e.g., maintenance phase). In some aspects, the first dose of the extension phase is administered about four weeks (e.g., four weeks) after administration of the last dose of the second phase (e.g., maintenance phase).

In some aspects in which the extension phase comprises administration of at least two doses of the anti-TL1A antibody, the time interval between each individual extension dose may be the same. In other aspects, the time interval between individual extension phase doses is not the same. The individual extension doses can be administered at least daily, at least one day apart, at least 1 week apart, at least 2 weeks apart, at least 3 weeks apart, at least 1 month apart, at least 2 months apart, at least 3 months apart, at least 4 months apart, at least 5 months apart, or at least 6 months apart. In one preferred aspect, the individual extension doses are administered one month apart.

In some aspects, the extension phase comprises administration of the anti-TL1A antibody every four weeks (Q4W).

In some aspects, the extension phase comprises administration of the anti-TL1A antibody every two weeks (Q2W).

In some aspects, the extension phase comprises (i) at least one interval in which the anti-TL1A antibody is administered every four weeks (Q4W) and (ii) at least one interval in which the anti-TL1A antibody is administered every two weeks (Q2W). In some aspects, the extension phase dosing frequency of a subject is increased from Q4W to Q2W based on observation of disease worsening.

The anti-TL1A antibody may be administered at a dose of, e.g., 1, 5, 10, 25, 50, 100, 150, 200, 250, 300, 350, 400, 450, 500, 550, 600, 650, 700, 750, 800, 850, 900, 950 or 1000 mg during the extension phase. In some aspects, the anti-TL1A antibody is administered at a dose of 50, 150 or 450 mg during the extension phase. In one aspect, the anti-TL1A antibody is administered at a dose of about 150 mg (e.g., at a dose of 150 mg) during the extension phase. In another aspect, the anti-TL1A antibody is administered at a dose of about 450 mg (e.g., at a dose of 450 mg) during the extension phase. Doses in the extension phase can be administered by any means (e.g., may be administered intravenously or subcutaneously). For example, in some aspects, in the extension phase, the anti-TL1A antibody is administered subcutaneously at a dose of 50, 150 or 450 mg. In some aspects, the anti-TL1A antibody is administered subcutaneously at a dose of 50, 150 or 450 mg every four weeks (Q4W).

In some aspects, in the extension phase, the anti-TL1A antibody is administered subcutaneously at a dose of about 150 mg (e.g., 150 mg) every four weeks. In some aspects, in the extension phase, the anti-TL1A antibody is administered subcutaneously at a dose of about 150 mg (e.g., 150 mg) every two weeks. In some aspects, each dose in the extension phase is about 150 mg (e.g., 150 mg), and the extension phase comprises (i) at least one interval in which the anti-TL1A antibody is subcutaneously administered every four weeks (Q4W) and (ii) at least one interval in which the anti-TL1A antibody is subcutaneously administered every two weeks (Q2W).

In some aspects, in the extension phase, the anti-TL1A antibody is administered subcutaneously at a dose of about 450 mg (e.g., 450 mg) every four weeks. In some aspects, in the extension phase, the anti-TL1A antibody is administered subcutaneously at a dose of about 450 mg (e.g., 450 mg) every two weeks. In some aspects, each dose in the extension phase is about 450 mg (e.g., 450 mg), and the extension phase comprises (i) at least one interval in which the anti-TL1A antibody is subcutaneously administered every four weeks (Q4W) and (ii) at least one interval in which the anti-TL1A antibody is subcutaneously administered every two weeks (Q2W).

In some aspects, the extension phase comprises administration of at least 2, 3, 4, 5, 6, 7, 8, 9, 10, 11, 12, 13, 14, 15, 16, 17, 18, 19, 20, 21, 22, 23, 24, 25, or more than 25 doses of the anti-TL1A antibody.

In some aspects, the one or more dosing cycles of the extension phase are administered to a patient who experienced disease worsening during the second phase (e.g., maintenance phase). In some aspects, the one or more dosing cycles of the extension phase are administered to a patient who experienced disease worsening during a dosing regimen comprising subcutaneous administration of the anti-TL1A antibody.

In some aspects, the one or more dosing cycles of the extension phase are administered to a patient who experienced disease worsening during the first phase (e.g., induction phase). In some aspects, the one or more dosing cycles of the extension phase are administered to a patient who experienced disease worsening during a dosing regimen comprising intravenous administration of the anti-TL1A antibody.

### D. Corticosteroid Tapering

In some aspects, the patient was receiving a stable corticosteroid dose (e.g., an oral corticosteroid, e.g., oral prednisone or an equivalent thereof, oral budesonide, or budesonide multi-matrix (MMX)) prior to the treatment, and the patient is tapered off of the corticosteroid during the second phase (e.g., maintenance phase) or extension phase (e.g., administration of the corticosteroid is discontinued during the second phase (e.g., maintenance phase) or extension phase). In some embodiments, the patient is tapered off of the corticosteroid during a dosing regimen comprising subcutaneous administration of the anti-TL1A antibody. For example, the dose of the corticosteroid may be tapered by 5 mg per week or 2.5 mg per week.

In some embodiments, the patient was receiving prednisone or an equivalent thereof administered orally at a dose of more than 10 mg per day in the induction phase, and the tapering comprises (i) tapering the dose by 5 mg per week until the patient is receiving a dose of 10 mg per day; and then (ii) tapering the dose by 2.5 mg/week until a dose of 0 mg per week is reached (e.g., tapering the dose by 2.5 mg per week for three weeks).

In some embodiments, the patient was receiving prednisone or an equivalent thereof administered orally at a dose of 10 mg or less per day in the induction phase, and the tapering comprises tapering the dose by 2.5 mg/week until a dose of 0 mg per week is reached (e.g., comprises tapering the dose by 2.5 mg per week for up to three weeks).

In some embodiments, the patient was receiving budesonide or budesonide MMX administered orally at a dose of 9 mg or less per day in the induction phase, and the tapering comprises the steps of (i) administering the corticosteroid at a dose of 9 mg every other day for two weeks; (ii) administering the corticosteroid at a dose of 9 mg every third day for two weeks; and (iii) discontinuing administration of the corticosteroid.

In some embodiments, the patient was receiving budesonide administered orally at a dose of 6 mg or less per day in the induction phase, and the tapering comprises tapering the dose to 3 mg/day for two weeks, and then discontinuing administration of the budesonide.

### E. Response to Treatment

### Clinical Response

Following the dosing regimen of the anti-TL1A antibody (e.g., following the first phase (e.g., induction dosing regimen) and/or the second phase (e.g., maintenance dosing regimen)), the patient may experience an improvement in signs and symptoms of IBD characterized by a clinical response.

In some aspects, the IBD is UC, and a clinical response is defined as a decrease in modified Mayo Score (mMS) of at least 2 points and 30% from baseline and either a decrease in rectal bleeding subscore (RBS) ≥1 or an RBS of 0 or 1.

In some aspects, the IBD is CD, and a clinical response is defined as a decrease in Crohn's disease activity index (CDAI) of at least 100 from baseline.

In some aspects, in a population of patients treated according to any one of the methods provided herein, the treating results in an increase in the proportion of patients who have achieved a clinical response at the end of the first phase (e.g., induction phase) as compared to a reference population. For example, in some aspects, the first phase (e.g., induction phase) has a duration of about 12 weeks, and the treating results in an increase in the proportion of patients who have achieved a clinical response at Week 12. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved a clinical response at Week 12. In some aspects, the proportion of patients in the population of patients who have achieved a clinical response at Week 12 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved a clinical response at Week 12 in the reference population. In some aspects, in a population of patients treated according to any one of the methods comprising a second phase (e.g., maintenance phase) provided herein, the treating results in an increase in the proportion of patients who have achieved a clinical response at the end of the second phase (e.g., maintenance phase) as compared to a reference population. For example, in some aspects, the dosing regimen has a duration of about 52 weeks, and the treating results in an increase in the proportion of patients who have achieved a clinical response at Week 52. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved a clinical response at Week 52. In some aspects, the proportion of patients in the population of patients who have achieved a clinical response at Week 52 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved a clinical response at Week 52 in the reference population.

In any of the methods provided herein, the reference population may be any population that serves as an appropriate control. For example, in some aspects, the reference population is a population of subjects having an IBD (e.g., UC (e.g., moderately or severely active UC) or CD (e.g., moderately or severely active CD)) who have not been treated with an anti-TL1A antibody (e.g., have been treated with a placebo).

### Endoscopic Response

Following the dosing regimen of the anti-TL1A antibody (e.g., following the first phase (e.g., induction dosing regimen) and/or the second phase (e.g., maintenance dosing regimen)), the patient may experience an improvement in signs and symptoms of IBD characterized by an endoscopic response.

In some aspects, the IBD is UC, and an endoscopic response is defined as a Mayo endoscopy subscore 0 or 1.

In some aspects, the IBD is CD, and an endoscopic response is defined as decrease in Simple Endoscopic Score for Crohn's disease (SES-CD) that is at least 50% lower than a baseline SES-CD.

In some aspects, in a population of patients treated according to any one of the methods provided herein, the treating results in an increase in the proportion of patients who have achieved an endoscopic response at the end of the first phase (e.g., induction phase) as compared to a reference population. For example, in some aspects, the first phase (e.g., induction phase) has a duration of about 12 weeks, and the treating results in an increase in the proportion of patients who have achieved an endoscopic response at Week 12. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved an endoscopic response at Week 12. In some aspects, the proportion of patients in the population of patients who have achieved an endoscopic response at Week 12 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved an endoscopic response at Week 12 in the reference population. In some aspects, in a population of patients treated according to any one of the methods comprising a second phase (e.g., maintenance phase) provided herein, the treating results in an increase in the proportion of patients who have achieved an endoscopic response at the end of the second phase (e.g., maintenance phase) as compared to a reference population. For example, in some aspects, the dosing regimen has a duration of about 52 weeks, and the treating results in an increase in the proportion of patients who have achieved an endoscopic response at Week 52. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved an endoscopic response at Week 52. In some aspects, the proportion of patients in the population of patients who have achieved an endoscopic response at Week 52 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved an endoscopic response at Week 52 in the reference population.

In any of the methods provided herein, the reference population may be any population that serves as an appropriate control. For example, in some aspects, the reference population is a population of subjects having an IBD (e.g., UC (e.g., moderately or severely active UC) or CD (e.g., moderately or severely active CD)) who have not been treated with an anti-TL1A antibody (e.g., have been treated with a placebo).

### Clinical Remission

Following the first dosing regimen (e.g., induction dosing regimen) and/or the second dosing regimen (e.g., maintenance dosing regimen), the patient may experience an improvement in signs and symptoms of IBD characterized by a clinical remission.

In some aspects, in a population of patients treated according to any one of the methods provided herein, the treating results in an increase in the proportion of patients who have achieved clinical remission at the end of the first phase (e.g., induction phase) as compared to a reference population. For example, in some aspects, the first phase (e.g., induction phase) has a duration of about 12 weeks, and the treating results in an increase in the proportion of patients who have achieved clinical remission at Week 12. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved clinical remission at Week 12. In some aspects, the proportion of patients in the population of patients who have achieved clinical remission at Week 12 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved clinical remission at Week 12 in the reference population.

In some aspects, in a population of patients treated according to any one of the methods comprising a second phase (e.g., maintenance phase) provided herein, the treating results in an increase in the proportion of patients who have achieved clinical remission at the end of the second phase (e.g., maintenance phase) as compared to a reference population. For example, in some aspects, the dosing regimen has a duration of about 52 weeks, and the treating results in an increase in the proportion of patients who have achieved clinical remission at Week 52. For example, in some aspects, at least 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved clinical remission at Week 52. In some aspects, the proportion of patients in the population of patients who have achieved clinical remission at Week 52 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved clinical remission at Week 52 in the reference population.

In some aspects, in a population of patients treated according to any one of the methods comprising a maintenance phase provided herein, the treating results in an increase in the proportion of patients who experience maintenance of remission throughout the second phase (e.g., maintenance phase) as compared to a reference population. For example, in some aspects, the first phase (e.g., induction phase) has a duration of about 12 weeks, the entire dosing regimen has a duration of about 52 weeks, and the treating results in an increase in the proportion of patients who are experiencing clinical remission at Week 12 and at Week 52.

In some aspects, the IBD is UC, and clinical remission is defined as a modified Mayo Score (mMS)≤2 with stool frequency subscore (SFS)=0 or 1, rectal bleeding subscore (RBS)=0, and endoscopic subscore(ES)=0 or 1. The mMS is evaluated as a composite based on three assessments, described in further detail below: stool frequency subscore (SFS), rectal bleeding subscore (RFS), and endoscopic subscore(ES). Each of these assessments has scoring that ranges from 0 to 3, with higher values indicating greater severity. mMS is calculated as the sum of SFS, RBS, and ES, with range 0-9.

SFS and RBS are calculated as an average over 7 days prior to the relevant timepoint.

SFS is quantified as follows:

With normal defined at baseline, with respect to each individual participant:
0=Normal number of stools for this patient.
1=1-2 more stools than normal.
2=3-4 more stools than normal.
3=5 or more stools than normal.

RBS is quantified as follows:

Representing the worst rectal bleeding observed by the participant that day:
0=No blood seen or no bowel movement.
1=Stool with streaks of blood.
2=Stool with more than streaks of blood.
3=Blood alone passed.

ES is quantified as follows:
0=Normal appearance of mucosa.
1=Mild disease (erythema, decreased vascular pattern, no friability).
2=Moderate disease (marked erythema, absent vascular pattern, friability, erosions).
3=Severe disease (spontaneous bleeding, ulceration).

In other aspects, the term “clinical remission” is based on 12-point total Mayo score: total Mayo score ≤2 with no individual subscore >1. In some aspects, Per Adapted (Modified) Mayo score is defined as endoscopic subscore=0 or 1, >=1-point decrease from baseline to achieve a stool frequency subscore=0 or 1, and rectal bleeding subscore=0. In some aspects, Per Adapted (Modified) Mayo score is defined as endoscopic subscore=0 or 1, a stool frequency subscore=0 or 1 with no increase from baseline, and rectal bleeding subscore=0.

In some aspects, the IBD is UC, and clinical remission is defined as a Crohn's disease activity index (CDAI) of less than 150.

### Endoscopic Remission

Following the first dosing regimen (e.g., induction dosing regimen) and/or the second dosing regimen (e.g., maintenance dosing regimen), the patient may experience an improvement in signs and symptoms of IBD characterized by an endoscopic remission. The term “endoscopic remission” refers to a Mayo endoscopy subscore of 0. In some aspects, the patient experiences endoscopic remission at or before the end of the first phase (e.g., induction phase), e.g., at or before Week 12 of treatment. In some aspects, in a population of patients treated according to any one of the methods provided herein, the treating results in an increase in the proportion of patients who have achieved endoscopic remission at the end of the first phase (e.g., induction phase) as compared to a reference population. In some aspects, the patient experiences endoscopic remission at or before the end of the second phase (e.g., maintenance phase), e.g., at or before Week 52 of treatment. In some aspects, in a population of patients treated according to any one of the methods provided herein, the treating results in an increase in the proportion of patients who have achieved endoscopic remission at the end of the second phase (e.g., maintenance phase) as compared to a reference population.

### Deep Remission

Following the first dosing regimen (e.g., induction dosing regimen) and/or the second dosing regimen (e.g., maintenance dosing regimen), the patient may experience an improvement in signs and symptoms of IBD characterized by a deep remission.

In some aspects, the IBD is UC, and the term “deep remission” refers to a total Mayo score of 2 points or lower, with no individual subscore exceeding 1 point and a 0 on both endoscopic and rectal bleeding subscore.

In some aspects, in a population of patients treated according to any one of the methods provided herein, the treating results in an increase in the proportion of patients who have achieved deep remission at the end of the first phase (e.g., induction phase) as compared to a reference population. In some aspects, the patient experiences deep remission at or before the end of the second phase (e.g., maintenance phase), e.g., at or before Week 52 of treatment. In some aspects, in a population of patients treated according to any one of the methods provided herein, the treating results in an increase in the proportion of patients who have achieved deep remission at the end of the second phase (e.g., maintenance phase) as compared to a reference population.

### Symptomatic Remission

Following the first dosing regimen (e.g., induction dosing regimen) and/or the second dosing regimen (e.g., maintenance dosing regimen), the patient may experience an improvement in signs and symptoms of IBD characterized by a symptomatic remission.

In some aspects, the IBD is UC, and the term “symptomatic remission” refers to a total Mayo score of 2 points or lower, with no individual subscore exceeding 1 point, and both rectal bleeding and stool frequency subscores of 0.

In some aspects, in a population of patients treated according to any one of the methods provided herein, the treating results in an increase in the proportion of patients who have achieved symptomatic remission at the end of the first phase (e.g., induction phase) as compared to a reference population. In some aspects, the patient experiences symptomatic remission at or before the end of the second phase (e.g., maintenance phase), e.g., at or before Week 52 of treatment. In some aspects, in a population of patients treated according to any one of the methods provided herein, the treating results in an increase in the proportion of patients who have achieved symptomatic remission at the end of the second phase (e.g., maintenance phase) as compared to a reference population.

### Endoscopic Improvement

Following the first dosing regimen (e.g., induction dosing regimen) and/or the second dosing regimen (e.g., maintenance dosing regimen), the patient may experience an improvement in signs and symptoms of IBD characterized by an endoscopic improvement.

In some aspects, the IBD is UC, and the term “endoscopic improvement” (“EI”) refers to a decrease of ≥1 point in Mayo endoscopy subscore or an absolute endoscopy score of ≤1. In some aspects, endoscopic improvement is defined as an endoscopic subscore of 0 or 1.

In some aspects, the patient experiences endoscopic improvement at or before the end of the first phase (e.g., induction phase), e.g., at or before Week 12 of treatment. In some aspects, in a population of patients treated according to any one of the methods provided herein, the treating results in an increase in the proportion of patients who have achieved endoscopic improvement at the end of the first phase (e.g., induction phase) as compared to a reference population. For example, in some aspects, the first phase (e.g., induction phase) has a duration of about 12 weeks, and the treating results in an increase in the proportion of patients who have achieved endoscopic improvement at Week 12. In some aspects, at least 1%, 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved endoscopic improvement at Week 12.

In some aspects, the proportion of patients in the population of patients who have achieved endoscopic improvement at Week 12 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved endoscopic improvement at Week 12 in the reference population.

In some aspects, the patient experiences endoscopic improvement at or before the end of the second phase (e.g., maintenance phase), e.g., at or before Week 52 of treatment. In some aspects, in a population of patients treated according to any one of the methods provided herein, the treating results in an increase in the proportion of patients who have achieved endoscopic improvement at the end of the second phase (e.g., maintenance phase) as compared to a reference population. In some aspects, at least 1%, 5%, 10%, 15%, 20%, 25%, 30%, 35%, 40%, 45%, 50%, 55%, 60%, 65%, 70%, 75%, 80%, 85%, 80%, 95%, or 99% of patients (e.g., 1-10%, 10-20%, 20-30%, 30-40%, 40-50%, 50-60%, 60-70%, 70-80%, 80-90%, or 90-100% of patients) in the population of patients have achieved endoscopic improvement at Week 52.

In some aspects, the proportion of patients in the population of patients who have achieved endoscopic improvement at Week 52 is at least 5%, 10%, 15%, 20%, 25%, 30%, 35%, 40%, 45%, 50%, 55%, 60%, 65%, 70%, 75%, 80%, 85%, 80%, 95%, or 99% greater than the proportion of patients who have achieved endoscopic improvement at Week 52 in the reference population.

### Improvement in Signs and Symptoms of IBD

Following the first dosing regimen (e.g., induction dosing regimen) and/or the second dosing regimen (e.g., maintenance dosing regimen), the patient may experience an improvement in signs and symptoms of IBD that are maintained while the patient receives the second dosing regimen (e.g., maintenance dosing regimen).

In some aspects of the disclosure, the first dosing regimen (e.g., induction dosing regimen) and/or the second dosing regimen (e.g., maintenance dosing regimen) with the anti-TL1A antibody effectively improves signs and symptoms of IBD by at least 12 weeks after starting of treatment with the anti-TL1A antibody. These improvement in signs and symptoms of IBD may be characterized by an improvement in the Mayo endoscopic subscore. The reduction of the patient's Mayo endoscopic subscore may be by at least 1, 2, or 3 or more integers.

The improvement in signs and symptoms of IBD may be characterized by the patient having a Mayo endoscopic subscore of 0 or 1, 2, or 3. The improvement in signs and symptoms of IBD may be characterized by the patient having a total Mayo score of 0, 1, 2, or 3. The improvement in signs and symptoms of IBD may be characterized by the patient having a Robarts Histopathology Index (RHI) of less than 5. The improvement in signs and symptoms of IBD may be characterized by the patient having a Geboes Index of less than 3.2.

The improvement in signs and symptoms of IBD may be maintained during the second dosing regimen (e.g., maintenance dosing regimen) for at least 2, 3, 4, 6, or 12 months.

### F. Prior Treatment

In some aspects, the patient has previously been treated with a therapy for IBD (e.g., a therapy for UC) and has experienced inadequate response to the therapy, loss of response to the therapy, and/or intolerance of the therapy.

In some aspects, the therapy was a conventional therapy for UC, e.g., comprised administration of a steroid, an immunomodulator, or an oral aminosalicylate (e.g., the patient has experienced inadequate response to, loss of response to, and/or intolerance of a steroid, an immunomodulator, and/or an oral aminosalicylate).

In some aspects, the therapy was an advanced therapy for UC, e.g., comprised administration of an anti-tumor necrosis factor (TNF) agent, an anti-integrin agent, an anti-IL12/IL23 agent, a Janus kinase (JAK) inhibitor, or a sphingosine-1-phosphate (S1P) inhibitor (e.g., the patient has experienced inadequate response to, loss of response to, and/or intolerance of an anti-TNF agent, an anti-integrin agent, an anti-IL 12/IL23 agent, a JAK inhibitor, or a S1P inhibitor). Thus, in some aspects, the patient had received prior advanced therapy at baseline (e.g., before administration of the first dose of the induction phase). In other aspects, the patient had not received prior advanced therapy at baseline.

In some aspects, the patient was receiving a corticosteroid therapy at baseline (e.g., before administration of the first dose of the induction phase). In other aspects, the patient was not receiving corticosteroid therapy at baseline.

### G. Inflammatory Bowel Diseases

### Ulcerative Colitis

In some aspects the IBD is ulcerative colitis (UC).

In some aspects, the UC is moderately to severely active UC. In some aspects, the moderately to severely active UC is UC having a modified Mayo score (mMS) of between 5 points and 9 points. In some aspects, the moderately to severely active UC is UC having a mMS of between 5 points and 9 points and a Mayo endoscopic score(ES) of 2 or 3. Thus, in some aspects, a patient to be treated according to the methods provided herein has UC with a mMS of 5 to 6 or has UC with a mMS of 7 to 9.

In some aspects, a patient having “moderate to severe” UC has 6 to 10 urgent bowel movements per day that are sometimes bloody. In some aspects, a patient having “severe” UC has more than 10 urgent bloody bowel movements per day. Patients with moderately and severely active UC might also have pain in the belly, tiredness, and weight loss. In some aspects, the disclosure features a method of treating UC (e.g., moderately to severely active UC) in a patient, the method comprising administering to the patient an effective amount of an anti-TL1A antibody in a dosing regimen comprising a first phase (e.g., an induction phase) and a second phase (e.g., a maintenance phase), wherein (a) the first phase (e.g., induction phase) comprises intravenous administration of only four doses of the anti-TL1A antibody at a dose of about 500 mg, wherein (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises subcutaneous administration of the anti-TL1A antibody every four weeks at a dose of about 150 mg. In some aspects, the disclosure features a method of treating UC (e.g., moderately to severely active UC) in a patient, the method comprising administering to the patient an effective amount of an anti-TL1A antibody in a dosing regimen comprising a first phase (e.g., an induction phase) and a second phase (e.g., a maintenance phase), wherein (a) the first phase (e.g., induction phase) comprises intravenous administration of only four doses of the anti-TL1A antibody at a dose of about 500 mg, wherein (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises subcutaneous administration of the anti-TL1A antibody every four weeks at a dose of about 450 mg. In some aspects, the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

### Crohn's Disease

In some aspects, the IBD is Crohn's disease (CD).

In some aspects, the CD is moderately to severely active CD. In some aspects, a patient having moderately to severely active CD (a) has a Simple Endoscopic Score for Crohn's Disease (SES-CD) of equal to or greater than 6; or (b) has isolated ileal disease only, and has an SES-CD of equal to or greater than 4. In some aspects, a patient having moderately to severely active CD (a) has a SES-CD of equal to or greater than 6 or (b) has isolated ileal disease only, and has an SES-CD of equal to or greater than 4, and has a Crohn's disease activity index (CDAI) that is at least 220 and is no greater than 450. Thus, in some aspects, a patient to be treated according to the methods provided herein has CD with a CDAI score of ≥220 and ≤450 and a SES-CD of ≥6 (or ≥4 for isolated ileal disease only).

In some aspects, the disclosure features a method of treating CD (e.g., moderately to severely active CD) in a patient, the method comprising administering to the patient an effective amount of an anti-TL1A antibody in a dosing regimen comprising a first phase (e.g., an induction phase) and a second phase (e.g., a maintenance phase), wherein (a) the first phase (e.g., induction phase) comprises intravenous administration of only four doses of the anti-TL1A antibody at a dose of about 500 mg, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises subcutaneous administration of the anti-TL1A antibody every four weeks at a dose of about 150 mg. In some aspects, the disclosure features a method of treating CD (e.g., moderately to severely active CD) in a patient, the method comprising administering to the patient an effective amount of an anti-TL1A antibody in a dosing regimen comprising a first phase (e.g., an induction phase) and a second phase (e.g., a maintenance phase), wherein (a) the first phase (e.g., induction phase) comprises intravenous administration of only four doses of the anti-TL1A antibody at a dose of about 500 mg, wherein: (i) the second dose of the anti-TL1A antibody is administered about two weeks after the first dose; (ii) the third dose of the anti-TL1A antibody is administered about four weeks after the second dose; and (iii) the fourth dose of the anti-TL1A antibody is administered about four weeks after the third dose; and (b) the second phase (e.g., maintenance phase) comprises subcutaneous administration of the anti-TL1A antibody every four weeks at a dose of about 450 mg. In some aspects, the anti-TL1A antibody comprises the following CDRs: (a) a CDR-H1 comprising the amino acid sequence of SEQ ID NO: 3; (b) a CDR-H2 comprising the amino acid sequence of SEQ ID NO: 4; (c) a CDR-H3 comprising the amino acid sequence of SEQ ID NO: 5; (d) a CDR-L1 comprising the amino acid sequence of SEQ ID NO: 6; (e) a CDR-L2 comprising the amino acid sequence of SEQ ID NO: 7; and (f) a CDR-L3 comprising the amino acid sequence of SEQ ID NO: 8. In some aspects, the anti-TL1A antibody is afimkibart.

### H. Patient is a TNFSF15 Haplotype B Non-Carrier

In some aspects, any of the methods, uses, or compositions for use provided herein comprise determining the TNFSF15 haplotype of a patient (e.g., comprise determining whether a patient is (a) a TNFSF15 haplotype B carrier or (b) a TNFSF15 haplotype B non-carrier). The methods, uses, or compositions for use may further include making a treatment decision on the basis of the patient's TNFSF15 haplotype. In some aspects, a patient identified for treatment with a method provided herein is a haplotype B non-carrier for TNFSF15 (e.g., has been determined to be a haplotype B non-carrier for TNFSF15).

Haplotype B is defined as a TNFSF15 gene region that has the nucleotides T/C/A/T/C at position 15,524; position 9,706; position-358; position-638; and position-12,506, respectively, based on the reference sequence GenBank NM_005118.2, wherein the first nucleotide of the exon 1 start site is designated as position 1 (haplotype positions 26/31/35/36/41, respectively, as provided in Yamazaki et al., Human Molecular Genetics, 14 (22): 3499-3506, 2005), in the TNFSF15 gene region, which encodes the TL1A gene (Table 1). A subject is identified as haplotype B if the subject is heterozygous or homozygous for haplotype B, i.e., one or both copies of the gene region are haplotype B (i.e., one or both of the TNFSF15 gene regions have the nucleotides T/C/A/T/C at haplotype positions 15,524; 9,706; −358; −638; and −12,506 (26/31/35/36/41).

TABLE 1
TNFSF15 gene region SNPs of interest

| | | Position number | | | | | |
|---|---|---|---|---|---|---|---|
| | | relative to GenBank | | | | | |
| | | NM_005118.2, | | | | | |
| | | wherein the first | | | | | |
| | Position number | nucleotide of the | NIH | | | | |
| | according | exon 1 start site | Reference | Position on | | | |
| | to Yamazaki | is designated | SNP (rs) | human genome | Reference | | Haplotype B |
| SNP | et al., 2005 | as position 1 | identifier | build hg38 | nucleotide | SNP | nucleotide |
| SNP 1 | 26 | 15,524 | rs3810936 | chr9: 114790605 | T | C | T |
| SNP 2 | 31 | 9,706 | rs6478108 | chr9: 114796423 | C | G or T | C |
| SNP 3 | 35 | −358 | rs6478109 | chr9: 114806486 | A | G | A |
| SNP 4 | 36 | −638 | rs7848647 | chr9: 114806766 | T | C | T |
| SNP 5 | 41 | −12,506 | rs7869487 | chr9: 114818634 | C | T | C |

Haplotype positions 15,524; 9,706; −358; −638; and −12,506 (26, 31, 35, 36 and 41) correspond to the sites of the SNPs rs3810936, rs6478108, rs6478109, rs7848647, and rs7869487, respectively, as identified in the NIH NCBI dbSNP database (see Table 1). Haplotype B comprises the reference nucleotide at each of these positions: thus, a haplotype B TNFSF15 gene region can be identified by the absence of each of SNPs rs3810936, rs6478108, rs6478109, rs7848647, and rs7869487 (i.e., the presence of the reference allele at each of these sites).

Haplotype B non-carrier subjects are defined as subjects that do not carry a haplotype B TNFSF15 gene region. For example, a haplotype B non-carrier subject may be homozygous for a non-haplotype B allele, e.g., haplotype A (e.g., may have the nucleotides C/T/G/C/T at haplotype positions 26/31/35/36/41). See, e.g., Michelsen et al., PLOS One, 4 (3): e4719, 2009).

### I. Combination Therapies

Anti-TL1A antibodies of the invention can be administered alone or used in a combination therapy. For instance, the combination therapy may include administering an anti-TL1A antibody according to any of the methods provided herein and administering at least one additional therapeutic agent (e.g. one, two, three, four, five, or six additional therapeutic agents). In one aspect, the combination therapy comprises administering an antibody of the invention and administering at least one additional therapeutic agent. Exemplary therapeutic agents that may be administered in combination with an anti-TL1A antibody are provided below. Further therapeutic agents that may be administered in combination with an anti-TL1A antibody are provided, e.g., in WO 2015/073580, US20220390463 A1, WO/2021/260577, and US20230235070 A1, which are hereby incorporated by reference in their entirety.

Such combination therapies encompass combined administration (where two or more therapeutic agents are included in the same or separate pharmaceutical composition(s)), and separate administration, in which case administration of the anti-TL1A antibody can occur prior to, simultaneously, and/or following, administration of the additional therapeutic agent or agents. In one aspect, administration of the anti-TL1A antibody and administration of an additional therapeutic agent occur within about one, two, three, four, five, or six days, within about one, two or three weeks, or within about one month, of each other. In one aspect, the antibody and additional therapeutic agent are both administered to the patient on Day 1 of the treatment.

In some aspects, the disclosure provides a pharmaceutical composition that comprises any of the anti-TL1A antibodies provided herein and at least one additional therapeutic agent, e.g., as described below.

### Combination Therapies for Patients Having UC

### Aminosalicylates

In some aspects, the IBD is ulcerative colitis (UC) (e.g., moderately to severely active UC) and the anti-TL1A antibody is administered in combination with an aminosalicylate, e.g., a 5-aminosalicylic acid (5-ASA).

As a general principle, aminosalicylates may be administered as a first-line treatment for mild to moderate UC. In some aspects, an anti-TL1A antibody is administered to a patient who has previously been treated with an aminosalicylate (e.g., a patient who experienced inadequate response, loss of response, and/or intolerance to an aminosalicylate). In other aspects, an anti-TL1A antibody is administered in combination with an aminosalicylate, e.g., in a patient who has not previously been treated with an aminosalicylate, and/or who has not experienced loss of response and/or intolerance to an aminosalicylate.

Exemplary aminosalicylates that may be used in the invention include, but are not limited to, mesalamine (also known as mesalazine or 5-ASA) e.g., APRISOR, ASACOL®, ASACOL® HD, CANASA®, DELZICOL®, FIV-ASA, LIALDA™, OCTASA®, PENTASAR, ROWASA®, SFROWASA®, or SALOFALK®; sulfasalazine, e.g., AZULFIDINE®; olsalazine (e.g., DIPENTUM®); and balsalazide (e.g., COLAZAL® or GIAZOR). In some aspects, the aminosalicylate is administered orally (i.e., is an oral aminosalicylate, e.g., oral 5-ASA or oral prednisone). In other aspects, the aminosalicylate is administered topically and/or rectally.

### Steroids

In some aspects, the IBD is UC (e.g., moderately to severely active UC) and the anti-TL1A antibody is administered in combination with a steroid (e.g., a corticosteroid).

As a general principle, corticosteroids may be administered to treat moderate to severe flares of UC (e.g., may be for short-term use). In some aspects, an anti-TL1A antibody is administered to a patient who has previously been treated with a corticosteroid (e.g., a patient who experienced inadequate response, loss of response, and/or intolerance to a corticosteroid). In some aspects, an anti-TL1A antibody is administered to a patient who is corticosteroid-dependent (e.g., is unable to taper a corticosteroid below a threshold dose without experiencing active disease), and/or who was corticosteroid-dependent prior to treatment with the anti-TL1A antibody. In some aspects, an anti-TL1A antibody is administered in combination with a corticosteroid, e.g., in a patient who has not previously been treated with a corticosteroid and/or who has not experienced loss of response and/or intolerance to a corticosteroid.

Exemplary corticosteroids that may be used in the invention include, but are not limited to, prednisone (e.g., RAYOS® or DELTASONE®), prednisolone, methylprednisolone (e.g., MEDROL®), and budesonide (e.g., oral budesonide) (e.g., UCERIS® or ENTOCORT® EC). In some aspects, the corticosteroid is administered orally (e.g., is oral prednisone or oral budesonide). In some aspects, the corticosteroid is administered intravenously or rectally.

### Immunomodulators

In some aspects, the IBD is UC (e.g., moderately to severely active UC) and the anti-TL1A antibody is administered in combination with an immunomodulator.

As a general principle, immunomodulators may be administered to achieve long-term immune suppression and/or may be steroid-sparing. In some aspects, an anti-TL1A antibody is administered to a patient who has previously been treated with an immunomodulator (e.g., a patient who experienced inadequate response, loss of response, and/or intolerance to an immunomodulator). In other aspects, an anti-TL1A antibody is administered in combination with an immunomodulator, e.g., in a patient who has not previously been treated with an immunomodulator and/or who has not experienced loss of response and/or intolerance to an immunomodulator.

Exemplary immunomodulators that may be used in the invention include, but are not limited to, azathioprine (AZA) (e.g., IMURAN®) (e.g., oral AZA), 6-mercaptopurine (6-MP) (e.g., PURINETHOL® or PURIXAN®) (e.g., oral 6-MP), methotrexate (MTX) (e.g., intramuscular or subcutaneously administered MTX), B-cell-depleting agents (e.g., natalizumab or rituximab), lymphocyte-depleting agents (e.g., alemtuzumab), cyclosporine, tacrolimus, sirolimus, and mycophenolate mofetil.

### Biologic Therapies

In some aspects, the IBD is UC (e.g., moderately to severely active UC) and the anti-TL1A antibody is administered in combination with a biologic therapy.

As a general principle, biologic therapies target inflammation pathways, and may be administered for treatment of moderate to severe UC. In some aspects, an anti-TL1A antibody is administered to a patient who has previously been treated with a biologic therapy (e.g., a patient who experienced inadequate response, loss of response, and/or intolerance to a biologic therapy). In other aspects, an anti-TL1A antibody is administered in combination with a biologic therapy, e.g., in a patient who has not previously been treated with a biologic therapy, and/or who has not experienced loss of response and/or intolerance to a biologic therapy.

Exemplary biologic therapies that may be used in the invention include, but are not limited to, tumor necrosis factor (TNF) inhibitors, e.g., infliximab (e.g., REMICADE®, INFLECTRA®, AVSOLA®, or RENFLEXIS®), adalimumab (e.g., HUMIRA®), and golimumab (e.g., SIMPONI®); integrin inhibitors, e.g., vedolizumab (e.g., ENTYVIO®); IL-12 and/or IL-23 inhibitors (e.g., IL-12/IL-23 inhibitors), e.g., ustekinumab (e.g., STELARA®), mirikizumab (e.g., OMVOH®), brazikumab, guselkumab (e.g., TREMFYA®), and risankizumab (e.g., SKYRIZI®); and death receptor 3 (DR3) inhibitors, e.g., SL-325.

### Anti-Tumor Necrosis Factor Agents

In some aspects, the IBD is UC (e.g., moderately to severely active UC) and the anti-TL1A antibody is administered in combination with an anti-tumor necrosis factor (TNF) agent (e.g., a TNF inhibitor, blocker, or modulator).

In some aspects, an anti-TL1A antibody is administered to a patient who has previously been treated with an anti-TNF agent (e.g., a patient who experienced inadequate response, loss of response, and/or intolerance to an anti-TNF agent). In other aspects, an anti-TL1A antibody is administered in combination with an anti-TNF agent, e.g., in a patient who has not previously been treated with an anti-TNF agent, and/or who has not experienced loss of response and/or intolerance to an anti-TNF agent.

Exemplary anti-TNF agents that may be used in the invention include, but are not limited to, infliximab (e.g., REMICADE®, INFLECTRA®, AVSOLAR, or RENFLEXIS®), adalimumab (e.g., HUMIRA®), golimumab (e.g., SIMPONI®), pegipanermin (e.g., XPRO™ or XPRO1595™), balinatunfib (SAR441566), benpyrine, R-7050, and apremilast (e.g., OTEZLA®). In some aspects, an anti-TNF agent that may be used in the invention is a small molecule inhibitor of TNF. Small molecule inhibitors of TNF include, but are not limited to pegipanermin (e.g., XPRO™ or XPRO1595™), balinatunfib (SAR441566), benpyrine, R-7050, and apremilast (e.g., OTEZLA®).

### Anti-Integrin Agents

In some aspects, the IBD is UC (e.g., moderately to severely active UC) and the anti-TL1A antibody is administered in combination with an anti-integrin agent (e.g., an integrin inhibitor).

In some aspects, an anti-TL1A antibody is administered to a patient who has previously been treated with an anti-integrin agent (e.g., a patient who experienced inadequate response, loss of response, and/or intolerance to an anti-integrin agent). In other aspects, an anti-TL1A antibody is administered in combination with an anti-integrin agent, e.g., in a patient who has not previously been treated with an anti-integrin agent, and/or who has not experienced loss of response and/or intolerance to an anti-integrin agent.

Exemplary anti-integrin agents that may be used in the invention include, but are not limited to, vedolizumab (e.g., ENTYVIO®).

### Anti-IL-12/IL-23 Agents

In some aspects, the IBD is UC (e.g., moderately to severely active UC) and the anti-TL1A antibody is administered in combination with an anti-IL-12/IL-23 agent (e.g., an IL-12/IL-23 inhibitor). In some aspects, an anti-TL1A antibody is administered to a patient who has previously been treated with an anti-IL-12/IL-23 agent (e.g., a patient who experienced inadequate response, loss of response, and/or intolerance to an anti-IL-12/IL-23 agent). In other aspects, an anti-TL1A antibody is administered in combination with an anti-IL-12/IL-23 agent, e.g., in a patient who has not previously been treated with an anti-IL-12/IL-23 agent, and/or who has not experienced loss of response and/or intolerance to an anti-IL-12/IL-23 agent.

Exemplary anti-IL-12/IL-23 agents that may be used in the invention include, but are not limited to, ustekinumab (e.g., STELARA®), mirikizumab (e.g., OMVOH®), brazikumab, guselkumab (e.g., TREMFYA®), risankizumab (e.g., SKYRIZI®), lisofylline (LSF), and icotrokinra (JNJ-2113). In some aspects, an anti-IL-12/IL-23 agent that may be used in the invention is a small molecule inhibitor of IL-12/IL-23. Small molecule inhibitors of IL-12/IL-23 include, but are not limited to lisofylline. In some aspects, the anti-IL-12/IL-23 agent is a synthetic peptide, (e.g., icotrokinra, e.g., orally administered icotrokinra).

### Death Receptor 3 Agents

In some aspects, the IBD is UC (e.g., moderately to severely active UC) and the anti-TL1A antibody is administered in combination with an anti-death receptor 3 (DR3) agent (e.g., a DR3 inhibitor). DR3 is also known as TNFRSF25.

In some aspects, an anti-TL1A antibody is administered to a patient who has previously been treated with an anti-DR3 agent (e.g., a patient who experienced inadequate response, loss of response, and/or intolerance to an anti-DR3 agent). In other aspects, an anti-TL1A antibody is administered in combination with an anti-DR3 agent, e.g., in a patient who has not previously been treated with an anti-DR3 agent, and/or who has not experienced loss of response and/or intolerance to an anti-DR3 agent.

Exemplary anti-DR3 agents that may be used in the invention include, but are not limited to, SL-325. In some aspects, an anti-DR3 agent that may be used in the invention is an anti-DR3 antibody (e.g., SL-325). In some aspects, an anti-DR3 agent that may be used in the invention is a small molecule inhibitor of DR3.

### Janus Kinase Inhibitors

In some aspects, the IBD is UC (e.g., moderately to severely active UC) and the anti-TL1A antibody is administered in combination with a Janus kinase (JAK) inhibitor or JAK modulator.

As a general principle, JAK inhibitors may be administered for treatment of moderate to severe UC, and may be administered orally. In some aspects, an anti-TL1A antibody is administered to a patient who has previously been treated with a JAK inhibitor (e.g., a patient who experienced inadequate response, loss of response, and/or intolerance to a JAK inhibitor). In other aspects, an anti-TL1A antibody is administered in combination with a JAK inhibitor, e.g., in a patient who has not previously been treated with a JAK inhibitor, and/or who has not experienced loss of response and/or intolerance to a JAK inhibitor.

Exemplary JAK inhibitors that may be used in the invention include, but are not limited to, tofacitinib (e.g., XELJANZ®), upadacitinib (e.g., RINVOQ®), peficitinib (e.g., SMYRAF®), and filgotinib (e.g., JYSELECA®).

### Sphingosine-1-Phosphate Receptor Modulators

In some aspects, the IBD is UC (e.g., moderately to severely active UC) and the anti-TL1A antibody is administered in combination with a sphingosine-1-phosphate (S1P) receptor modulator. As a general principle, S1P receptor modulators may be administered as an oral therapy for moderate UC. In some aspects, an anti-TL1A antibody is administered to a patient who has previously been treated with a S1P receptor modulator (e.g., a patient who experienced inadequate response, loss of response, and/or intolerance to a S1P receptor modulator). In other aspects, an anti-TL1A antibody is administered in combination with a S1P receptor modulator, e.g., in a patient who has not previously been treated with a S1P receptor modulator, and/or who has not experienced loss of response and/or intolerance to a S1P receptor modulator.

Exemplary S1P receptor modulators that may be used in the invention include, but are not limited to, ozanimod (e.g., ZEPOSIA®), etrasimod (e.g., VELSIPITY®), and amiselimod.

miR-124 Regulators

In some aspects, the IBD is UC (e.g., moderately to severely active UC) and the anti-TL1A antibody is administered in combination with an agent that regulates (e.g., upregulates) miR-124, e.g., obefazimod (ABX464).

### Antibiotics

In some aspects, the IBD is UC (e.g., moderately to severely active UC) and the anti-TL1A antibody is administered in combination with an antibiotic (e.g., an orally or intravenously administered antibiotic).

As a general principle, antibiotics may be administered to treat complications of UC, e.g., pouchitis or secondary infections. Exemplary antibiotics that may be used in the invention include, but are not limited to, ciprofloxacin, metronidazole, azithromycin, and erythromycin.

### Supportive and Adjunctive Treatments

In some aspects, the IBD is UC (e.g., moderately to severely active UC) and the anti-TL1A antibody is administered in combination with a supportive or adjunctive treatment.

Exemplary supportive or adjunctive treatments that may be used in the invention include, but are not limited to, probiotics (e.g., VSL #3® or VISBIOME®), prebiotics and/or dietary fibers (e.g., pectin, inulin, psyllium, fructo-oligosaccharides, or lactulose), iron supplements for the treatment of anemia, antihistamines, and pain management agents (e.g., acetaminophen). In some aspects, acetaminophen is preferred over NSAIDs for pain management.

### Microbiome-Based Therapies

In some aspects, the IBD is UC (e.g., moderately to severely active UC) and the anti-TL1A antibody is administered in combination with a microbiome-based therapy.

Exemplary microbiome-based therapies that may be used in the invention include, but are not limited to, microbiome-modulating therapies (e.g., SER-287) and fecal microbiota transplant (FMT)-based treatment (e.g., RBX2660).

### Stem Cell and Gene Therapies

In some aspects, the IBD is UC (e.g., moderately to severely active UC) and the anti-TL1A antibody is administered in combination with a stem cell therapy or a gene therapy.

Exemplary stem cell or gene therapies that may be used in the invention include, but are not limited to, therapies for UC-related tissue repair (e.g., Mesenchymal Stem Cell (MSC) therapy), gene therapies targeting inflammation reduction, and therapies for autoimmune control (e.g., CAR-T cell therapy).

### Herbal Supplements and Botanical Remedies

In some aspects, the IBD is UC (e.g., moderately to severely active UC) and the anti-TL1A antibody is administered in combination with an herbal supplement and/or a botanical remedy (e.g., a plant, a plant preparation (e.g., a food product, tea, tincture, poultice, cream, oil, salve, balm, or lotion comprising one or more plant-derived components), or an extract or other natural product from a plant).

As a general principle, herbal supplements and botanical remedies with anti-inflammatory, antioxidant, antidiarrheal, antimicrobial, digestion-improving, and/or demulcent properties may be administered to manage, alleviate, and/or improve symptoms associated with UC.

Exemplary plants, plant preparations, plant extracts, and products that may be used in the invention include, but are not limited to, curcumin or a plant or plant preparation comprising the same (e.g., turmeric (Curcuma longa)), Aloe vera (e.g., Aloe vera juice or gel), chamomile (e.g., Matricaria chamomilla), wheatgrass, myrrh or a plant or plant preparation comprising the same (e.g., Commiphora myrrha), berberine or a plant or plant preparation comprising the same (e.g., goldenseal or barberry), and slippery elm (Ulmus rubra) or a preparation thereof (e.g., bark or leaves); and/or a traditional medicine (e.g., Chinese medicine). In some aspects, the herbal supplement or botanical remedy is administered orally (e.g., as an oral vitamin, food product, tea, or tincture). In other aspects, the herbal supplement or botanical remedy is administered topically (e.g., as a poultice, cream, oil, salve, balm, or lotion) and/or rectally (e.g., as an herbal enema).

### Vagus Nerve Stimulation

In some aspects, the IBD is UC (e.g., moderately to severely active UC) and the anti-TL1A antibody is administered in combination with a vagus nerve stimulation (VNS) therapy, e.g., a VNS device or technique.

Exemplary VNS therapies that may be used in the invention include, but are not limited to, implanted or transcutaneous neurostimulator devices (e.g., VNS THERAPY® system or GAMMACORE® device). VNS therapies further include non-invasive vagus nerve stimulation techniques (e.g., diaphragmatic breathing, progressive muscle relaxation, and massage).

### Combination Therapies for Patients Having Crohn's Disease

### Aminosalicylates

In some aspects, the IBD is Crohn's disease (CD) (e.g., moderately to severely active CD) and the anti-TL1A antibody is administered in combination with an aminosalicylate, e.g., a 5-aminosalicylic acid (5-ASA).

As a general principle, aminosalicylates may be administered for treatment of mild to moderate CD. In some aspects, an anti-TL1A antibody is administered to a patient who has previously been treated with an aminosalicylate (e.g., a patient who experienced inadequate response, loss of response, and/or intolerance to an aminosalicylate). In other aspects, an anti-TL1A antibody is administered in combination with an aminosalicylate, e.g., in a patient who has not previously been treated with an aminosalicylate and/or who has not experienced loss of response and/or intolerance to an aminosalicylate.

Exemplary aminosalicylates that may be used in the invention include, but are not limited to, mesalamine (also known as mesalazine or 5-ASA) e.g., APRISOR, ASACOL®, ASACOL® HD, CANASA®, DELZICOL®, FIV-ASA, LIALDA™, OCTASA®, PENTASAR, ROWASA®, SFROWASA®, or SALOFALK®; sulfasalazine, e.g., AZULFIDINE®; olsalazine (e.g., DIPENTUM®); and balsalazide (e.g., COLAZAL® or GIAZOR). In some aspects, the aminosalicylate is administered orally (i.e., is an oral aminosalicylate, e.g., oral 5-ASA or oral prednisone). In other aspects, the aminosalicylate is administered topically and/or rectally.

### Steroids

In some aspects, the IBD is CD (e.g., moderately to severely active CD) and the anti-TL1A antibody is administered in combination with a steroid (e.g., a corticosteroid).

As a general principle, corticosteroids may be administered to treat moderate to severe flares of CD (e.g., may be for short-term use). In some aspects, an anti-TL1A antibody is administered to a patient who has previously been treated with a corticosteroid (e.g., a patient who experienced inadequate response, loss of response, and/or intolerance to a corticosteroid). In some aspects, an anti-TL1A antibody is administered to a patient who is corticosteroid-dependent (e.g., is unable to taper a corticosteroid below a threshold dose without experiencing active disease), and/or who was corticosteroid-dependent prior to treatment with the anti-TL1A antibody. In some aspects, an anti-TL1A antibody is administered in combination with a corticosteroid, e.g., in a patient who has not previously been treated with a corticosteroid and/or who has not experienced loss of response and/or intolerance to a corticosteroid.

Exemplary corticosteroids that may be used in the invention include, but are not limited to, prednisone (e.g., RAYOS® or DELTASONE®), prednisolone, methylprednisolone (e.g., MEDROL®), and budesonide (e.g., oral budesonide) (e.g., UCERIS® or ENTOCORT® EC). In some aspects, the corticosteroid is administered orally (e.g., is oral prednisone or oral budesonide). In some aspects, the corticosteroid is administered intravenously or rectally.

### Immunomodulators

In some aspects, the IBD is CD (e.g., moderately to severely active CD) and the anti-TL1A antibody is administered in combination with an immunomodulator.

As a general principle, immunomodulators may be administered to achieve long-term immune suppression and/or may be steroid-sparing. In some aspects, an anti-TL1A antibody is administered to a patient who has previously been treated with an immunomodulator (e.g., a patient who experienced inadequate response, loss of response, and/or intolerance to an immunomodulator). In other aspects, an anti-TL1A antibody is administered in combination with an immunomodulator, e.g., in a patient who has not previously been treated with an immunomodulator and/or who has not experienced loss of response and/or intolerance to an immunomodulator.

Exemplary immunomodulators that may be used in the invention include, but are not limited to, azathioprine (AZA) (e.g., IMURAN®) (e.g., oral AZA), 6-mercaptopurine (6-MP) (e.g., PURINETHOL® or PURIXAN®) (e.g., oral 6-MP), methotrexate (MTX) (e.g., intramuscular or subcutaneously administered MTX), B-cell-depleting agents (e.g., natalizumab or rituximab), lymphocyte-depleting agents (e.g., alemtuzumab), cyclosporine, tacrolimus, sirolimus, and mycophenolate mofetil.

### Biologic Therapies

In some aspects, the IBD is CD (e.g., moderately to severely active CD) and the anti-TL1A antibody is administered in combination with a biologic therapy.

As a general principle, biologic therapies target inflammation pathways, and may be administered for treatment of moderate to severe CD. In some aspects, an anti-TL1A antibody is administered to a patient who has previously been treated with a biologic therapy (e.g., a patient who experienced inadequate response, loss of response, and/or intolerance to a biologic therapy). In other aspects, an anti-TL1A antibody is administered in combination with a biologic therapy, e.g., in a patient who has not previously been treated with a biologic therapy and/or who has not experienced loss of response and/or intolerance to a biologic therapy.

Exemplary biologic therapies that may be used in the invention include, but are not limited to, tumor necrosis factor (TNF) inhibitors, e.g., infliximab (e.g., REMICADE®, INFLECTRA®, AVSOLA®, or RENFLEXIS®), adalimumab (e.g., HUMIRA®), and certolizumab pegol (e.g., CIMZIA®); integrin inhibitors, e.g., vedolizumab (e.g., ENTYVIO®); IL-12 and/or IL-23 inhibitors (e.g., IL-12/IL-23 inhibitors), e.g., ustekinumab (e.g., STELARA®), mirikizumab (e.g., OMVOH®), brazikumab, guselkumab (e.g., TREMFYA®), risankizumab (e.g., SKYRIZI®), and tildrakizumab; and death receptor 3 (DR3) inhibitors, e.g., SL-325.

### Anti-Tumor Necrosis Factor Agents

In some aspects, the IBD is CD (e.g., moderately to severely active CD) and the anti-TL1A antibody is administered in combination with an anti-tumor necrosis factor (TNF) agent (e.g., a TNF inhibitor, blocker, or modulator).

In some aspects, an anti-TL1A antibody is administered to a patient who has previously been treated with an anti-TNF agent (e.g., a patient who experienced inadequate response, loss of response, and/or intolerance to an anti-TNF agent). In other aspects, an anti-TL1A antibody is administered in combination with an anti-TNF agent, e.g., in a patient who has not previously been treated with an anti-TNF agent, and/or who has not experienced loss of response and/or intolerance to an anti-TNF agent.

Exemplary anti-TNF agents that may be used in the invention include, but are not limited to, infliximab (e.g., REMICADE®, INFLECTRA®, AVSOLAR, or RENFLEXIS®), adalimumab (e.g., HUMIRA®), certolizumab pegol (e.g., CIMZIA®), pegipanermin (e.g., XPRO™ or XPRO1595™), balinatunfib (SAR441566), benpyrine, R-7050, and apremilast (e.g., OTEZLA®). In some aspects, an anti-TNF agent that may be used in the invention is a small molecule inhibitor of TNF. Small molecule inhibitors of TNF include, but are not limited to pegipanermin (e.g., XPRO™ or XPRO1595™), balinatunfib (SAR441566), benpyrine, R-7050, and apremilast (e.g., OTEZLA®).

### Anti-Integrin Agents

In some aspects, the IBD is UC (e.g., moderately to severely active UC) and the anti-TL1A antibody is administered in combination with an anti-integrin agent (e.g., an integrin inhibitor).

In some aspects, an anti-TL1A antibody is administered to a patient who has previously been treated with an anti-integrin agent (e.g., a patient who experienced inadequate response, loss of response, and/or intolerance to an anti-integrin agent). In other aspects, an anti-TL1A antibody is administered in combination with an anti-integrin agent, e.g., in a patient who has not previously been treated with an anti-integrin agent, and/or who has not experienced loss of response and/or intolerance to an anti-integrin agent.

Exemplary anti-integrin agents that may be used in the invention include, but are not limited to, vedolizumab (e.g., ENTYVIO®).

### Anti-IL-12/IL-23 Agents

In some aspects, the IBD is CD (e.g., moderately to severely active CD) and the anti-TL1A antibody is administered in combination with an anti-IL-12/IL-23 agent (e.g., an IL-12/IL-23 inhibitor).

In some aspects, an anti-TL1A antibody is administered to a patient who has previously been treated with an anti-IL-12/IL-23 agent (e.g., a patient who experienced inadequate response, loss of response, and/or intolerance to an anti-IL-12/IL-23 agent). In other aspects, an anti-TL1A antibody is administered in combination with an anti-IL-12/IL-23 agent, e.g., in a patient who has not previously been treated with an anti-IL-12/IL-23 agent, and/or who has not experienced loss of response and/or intolerance to an anti-IL-12/IL-23 agent.

Exemplary anti-IL-12/IL-23 agents that may be used in the invention include, but are not limited to, ustekinumab (e.g., STELARA®), mirikizumab (e.g., OMVOH®), brazikumab, guselkumab (e.g., TREMFYA®), risankizumab (e.g., SKYRIZI®), tildrakizumab, lisofylline (LSF), and icotrokinra (JNJ-2113). In some aspects, an anti-IL-12/IL-23 agent that may be used in the invention is a small molecule inhibitor of IL-12/IL-23. Small molecule inhibitors of IL-12/IL-23 include, but are not limited to lisofylline. In some aspects, the anti-IL-12/IL-23 agent is a synthetic peptide (e.g., icotrokinra, e.g., orally administered icotrokinra).

### Death Receptor 3 Agents

In some aspects, the IBD is CD (e.g., moderately to severely active CD) and the anti-TL1A antibody is administered in combination with an anti-death receptor 3 (DR3) agent (e.g., a DR3 inhibitor).

In some aspects, an anti-TL1A antibody is administered to a patient who has previously been treated with an anti-DR3 agent (e.g., a patient who experienced inadequate response, loss of response, and/or intolerance to an anti-DR3 agent). In other aspects, an anti-TL1A antibody is administered in combination with an anti-DR3 agent, e.g., in a patient who has not previously been treated with an anti-DR3 agent, and/or who has not experienced loss of response and/or intolerance to an anti-DR3 agent.

Exemplary anti-DR3 agents that may be used in the invention include, but are not limited to, SL-325. In some aspects, an anti-DR3 agent that may be used in the invention is an anti-DR3 antibody (e.g., SL-325). In some aspects, an anti-DR3 agent that may be used in the invention is a small molecule inhibitor of DR3.

### Janus Kinase Inhibitors

In some aspects, the IBD is CD (e.g., moderately to severely active CD) and the anti-TL1A antibody is administered in combination with a Janus kinase (JAK) inhibitor or JAK modulator.

As a general principle, JAK inhibitors may be administered for treatment of moderate to severe CD, and may be administered orally. In some aspects, an anti-TL1A antibody is administered to a patient who has previously been treated with a JAK inhibitor (e.g., a patient who experienced inadequate response, loss of response, and/or intolerance to a JAK inhibitor). In other aspects, an anti-TL1A antibody is administered in combination with a JAK inhibitor, e.g., in a patient who has not previously been treated with a JAK inhibitor and/or who has not experienced loss of response and/or intolerance to a JAK inhibitor.

Exemplary JAK inhibitors that may be used in the invention include, but are not limited to, tofacitinib (e.g., XELJANZ®), upadacitinib (e.g., RINVOQ®), peficitinib (e.g., SMYRAF®), and filgotinib (e.g., JYSELECA®).

### Sphingosine-1-Phosphate Receptor Modulators

In some aspects, the IBD is CD (e.g., moderately to severely active CD) and the anti-TL1A antibody is administered in combination with a sphingosine-1-phosphate (S1P) receptor modulator.

As a general principle, S1P receptor modulators may be administered as an oral therapy for moderate CD. In some aspects, an anti-TL1A antibody is administered to a patient who has previously been treated with a S1P receptor modulator (e.g., a patient who experienced inadequate response, loss of response, and/or intolerance to a S1P receptor modulator). In other aspects, an anti-TL1A antibody is administered in combination with a S1P receptor modulator, e.g., in a patient who has not previously been treated with a S1P receptor modulator and/or who has not experienced loss of response and/or intolerance to a S1P receptor modulator.

Exemplary S1P receptor modulators that may be used in the invention include, but are not limited to, ozanimod (e.g., ZEPOSIA®), etrasimod (e.g., VELSIPITY®), and amiselimod.

miR-124 Regulators

In some aspects, the IBD is UC (e.g., moderately to severely active UC) and the anti-TL1A antibody is administered in combination with an agent that regulates (e.g., upregulates) miR-124, e.g., obefazimod (ABX464).

### Antibiotics

In some aspects, the IBD is CD (e.g., moderately to severely active CD) and the anti-TL1A antibody is administered in combination with an antibiotic (e.g., an orally or intravenously administered antibiotic).

As a general principle, antibiotics may be administered to treat complications of CD, e.g., infections, abcesses, or fistulas. Exemplary antibiotics that may be used in the invention include, but are not limited to, ciprofloxacin, metronidazole, azithromycin, erythromycin, and rifaximin (e.g., rifamixin used for small intestinal bacterial overgrowth or complications).

### Supportive and Adjunctive Treatments

In some aspects, the IBD is CD (e.g., moderately to severely active CD) and the anti-TL1A antibody is administered in combination with a supportive or adjunctive treatment.

Exemplary supportive or adjunctive treatments that may be used in the invention include, but are not limited to, probiotics (e.g., agents that modulate the gut microbiome, e.g., VSL #3® or VISBIOME®), prebiotics and/or dietary fibers (e.g., pectin, inulin, psyllium, fructo-oligosaccharides, or lactulose), iron supplements for the treatment of anemia (e.g., anemia caused by chronic inflammation or blood loss), antihistamines, and pain management agents (e.g., acetaminophen). In some aspects, acetaminophen is preferred over NSAIDs for pain management, e.g., because NSAIDs may worsen CD symptoms.

### Microbiome-Based Therapies

In some aspects, the IBD is CD (e.g., moderately to severely active CD) and the anti-TL1A antibody is administered in combination with a microbiome-based therapy.

Exemplary microbiome-based therapies that may be used in the invention include, but are not limited to, microbiome-modulating therapies (e.g., SER-287) and fecal microbiota transplant (FMT)-based treatment (e.g., RBX2660).

### Stem Cell and Gene Therapies

In some aspects, the IBD is CD (e.g., moderately to severely active CD) and the anti-TL1A antibody is administered in combination with a stem cell therapy or a gene therapy.

Exemplary stem cell or gene therapies that may be used in the invention include, but are not limited to, therapies for regeneration and inflammation control in CD (e.g., Mesenchymal Stem Cell (MSC) therapy), gene therapies targeting inflammation reduction, and therapies for autoimmune control (e.g., CAR-T cell therapy).

### Herbal Supplements and Botanical Remedies

In some aspects, the IBD is CD (e.g., moderately to severely active CD) and the anti-TL1A antibody is administered in combination with an herbal supplement and/or a botanical remedy (e.g., a plant, a plant preparation (e.g., a food product, tea, tincture, poultice, cream, oil, salve, balm, or lotion comprising one or more plant-derived components), or an extract or other natural product from a plant).

As a general principle, herbal supplements and botanical remedies with anti-inflammatory, antioxidant, antidiarrheal, antimicrobial, digestion-improving, and/or demulcent properties may be administered to manage, alleviate, and/or improve symptoms associated with CD.

Exemplary plants, plant preparations, plant extracts, and products that may be used in the invention include, but are not limited to, curcumin or a plant or plant preparation comprising the same (e.g., turmeric (Curcuma longa)), boswellia (e.g., Boswellia serrata), chamomile (e.g., Matricaria chamomilla), myrrh or a plant or plant preparation comprising the same (e.g., Commiphora myrrha), berberine or a plant or plant preparation comprising the same (e.g., goldenseal or barberry), and slippery elm (Ulmus rubra) or a preparation thereof (e.g., bark or leaves); and/or a traditional medicine (e.g., Chinese medicine). In some aspects, the herbal supplement or botanical remedy is administered orally (e.g., as an oral vitamin, food product, tea, or tincture). In other aspects, the herbal supplement or botanical remedy is administered topically (e.g., as a poultice, cream, oil, salve, balm, or lotion) and/or rectally (e.g., as an herbal enema).

### Vagus Nerve Stimulation

In some aspects, the IBD is UC (e.g., moderately to severely active UC) and the anti-TL1A antibody is administered in combination with a vagus nerve stimulation (VNS) therapy, e.g., a VNS device or technique.

Exemplary VNS therapies that may be used in the invention include, but are not limited to, implanted or transcutaneous neurostimulator devices (e.g., VNS THERAPY® system or GAMMACORE® device). VNS therapies further include non-invasive vagus nerve stimulation techniques (e.g., diaphragmatic breathing, progressive muscle relaxation, and massage).

### III. Anti-TL1A Antibodies

The methods of the present disclosure include the administration of an anti-TL1A antibody. Exemplary anti-TL1A antibodies of the disclosure are set forth in Table 2.

TABLE 2
Sequences of Exemplary Antibodies of the Disclosure

| SEQ ID | | |
|---|---|---|
| number | Description | Amino Acid Sequence |
| 1 | Afimkibart | QVQLVQSGAEVKKPGASVKVSCKASGYDFTYYGISWVRQAPGQG |
| | heavy chain | LEWMGWISTYNGNTHYARMLQGRVTMTTDTSTRTAYMELRSLRS |
| | variable | DDTAVYYCARENYYGSGAYRGGMDVWGQGTTVTVSS |
| | domain (VH) | |
| 2 | Afimkibart light | EIVLTQSPATLSLSPGERATLSCRASQSVSSYLAWYQQKPGQAPR |
| | chain variable | LLIYDASNRATGIPARFSGSGSGTDFTLTISSLEPEDFAVYYCQQR |
| | domain (VL) | SNWPWTFGQGTKVEIK |
| 3 | Afimkibart | YYGIS |
| | CDR-H1 | |
| 4 | Afimkibart | WISTYNGNTHYARMLQG |
| | CDR-H2 | |
| 5 | Afimkibart | ENYYGSGAYRGGMDV |
| | CDR-H3 | |
| 6 | Afimkibart | RASQSVSSYLA |
| | CDR-L1 | |
| 7 | Afimkibart | DASNRAT |
| | CDR-L2 | |
| 8 | Afimkibart | QQRSNWPWT |
| | CDR-L3 | |
| 9 | Afimkibart | QVQLVQSGAEVKKPGASVKVSCKASGYDFTYYGISWVRQAPGQG |
| | heavy chain | LEWMGWISTYNGNTHYARMLQGRVTMTTDTSTRTAYMELRSLRS |
| | (HC) | DDTAVYYCARENYYGSGAYRGGMDVWGQGTTVTVSSASTKGPS |
| | | VFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKV |
| | | EPKSCDKTHTCPPCPAPEAAGAPSVFLFPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALPAPIEKTISKAKGQPREPQVYTL |
| | | PPSREEMTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPG |
| 10 | Afimkibart light | EIVLTQSPATLSLSPGERATLSCRASQSVSSYLAWYQQKPGQAPR |
| | chain (LC) | LLIYDASNRATGIPARFSGSGSGTDFTLTISSLEPEDFAVYYCQQR |
| | | SNWPWTFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| 11 | Afimkibart HC | QVQLVQSGAEVKKPGASVKVSCKASGYDFTYYGISWVRQAPGQG |
| | (with C-terminal | LEWMGWISTYNGNTHYARMLQGRVTMTTDTSTRTAYMELRSLRS |
| | lysine) | DDTAVYYCARENYYGSGAYRGGMDVWGQGTTVTVSSASTKGPS |
| | | VFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKKV |
| | | EPKSCDKTHTCPPCPAPEAAGAPSVFLFPPKPKDTLMISRTPEVTC |
| | | VVVDVSHEDPEVKFNWYVDGVEVHNAKTKPREEQYNSTYRVVSV |
| | | LTVLHQDWLNGKEYKCKVSNKALPAPIEKTISKAKGQPREPQVYTL |
| | | PPSREEMTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 12 | Afimkibart long | GYDFTYYGIS |
| | version of | |
| | CDR-H1 | |
| 13 | Afimkibart | QVQLVQSGAEVKKPGASVKVSCKASGYDFT |
| | heavy chain | |
| | framework | |
| | region 1 | |
| | (HFR1) | |
| 14 | Afimkibart | WVRQAPGQGLEWMG |
| | HFR2 | |
| 15 | Afimkibart | RVTMTTDTSTRTAYMELRSLRSDDTAVYYCAR |
| | HFR3 | |
| 16 | Afimkibart | WGQGTTVTVSS |
| | HFR4 | |
| 17 | Afimkibart light | EIVLTQSPATLSLSPGERATLSC |
| | chain | |
| | framework | |
| | region 1 (LFR1) | |
| 18 | Afimkibart | WYQQKPGQAPRLLIY |
| | LFR2 | |
| 19 | Afimkibart | GIPARFSGSGSGTDFTLTISSLEPEDFAVYYC |
| | LFR3 | |
| 20 | Afimkibart | FGQGTKVEIKRTV |
| | LFR4 | |
| 21 | Afimkibart short | QVQLVQSGAEVKKPGASVKVSCKAS |
| | version of | |
| | HFR1 | |

In some aspects of the disclosure, the anti-TL1A antibody comprises a CDR-H1 comprising the amino acid sequence of SEQ ID NO: 3, a CDR-H2 comprising the amino acid sequence of SEQ ID NO: 4, a CDR-H3 comprising the amino acid sequence of SEQ ID NO: 5, a CDR-L1 comprising the amino acid sequence of SEQ ID NO: 6, a CDR-L2 comprising the amino acid sequence of SEQ ID NO: 7, and a CDR-L3 comprising the amino acid sequence of SEQ ID NO: 8. In some aspects, the anti-TL1A antibody comprises one, two, three, four, five, six, seven, or all eight of the framework region sequences shown in SEQ ID NOs: 13-20.

In some aspects of the disclosure, the anti-TL1A antibody comprises a CDR-H1 comprising the amino acid sequence of SEQ ID NO: 12, a CDR-H2 comprising the amino acid sequence of SEQ ID NO: 4, a CDR-H3 comprising the amino acid sequence of SEQ ID NO: 5, a CDR-L1 comprising the amino acid sequence of SEQ ID NO: 6, a CDR-L2 comprising the amino acid sequence of SEQ ID NO: 7, and a CDR-L3 comprising the amino acid sequence of SEQ ID NO: 8. In some aspects, the anti-TL1A antibody comprises one, two, three, four, five, six, seven, or all eight of the framework region sequences shown in SEQ ID NOs: 14-21.

In some aspects of the disclosure, the anti-TL1A antibody comprises a heavy chain variable domain (VH) having at least 95% sequence identity to the amino acid sequence of SEQ ID NO: 1 (e.g., having at least 96%, 97%, 98%, or 99% identity to SEQ ID NO: 1) and/or comprises a light chain variable (VL) domain comprising an amino acid sequence having at least 95% sequence identity to the amino acid sequence of SEQ ID NO: 2 (e.g., having at least 96%, 97%, 98%, or 99% identity to SEQ ID NO: 2).

In some aspects of the disclosure, the anti-TL1A antibody comprises a VH comprising the amino acid sequence of SEQ ID NO: 1 and/or comprises a VL domain comprising the amino acid sequence of SEQ ID NO: 2.

In some aspects of the disclosure, the anti-TL1A antibody comprises a VH having the sequence shown in SEQ ID NO: 1 and a VL having the sequence shown in SEQ ID NO: 2.

In some aspects of the disclosure, the anti-TL1A antibody comprises (a) a heavy chain comprising an amino acid sequence having at least 95% sequence identity to the amino acid sequence of SEQ ID NO: 9 or SEQ ID NO: 11 (e.g., having at least 96%, 97%, 98%, or 99% identity to SEQ ID NO: 9 or SEQ ID NO: 11); and/or (b) a light chain comprising an amino acid sequence having at least 95% sequence identity to the amino acid sequence of SEQ ID NO: 10 (e.g., having at least 96%, 97%, 98%, or 99% identity to SEQ ID NO: 10).

In some aspects, the anti-TL1A antibody comprises (a) a heavy chain comprising the amino acid sequence of SEQ ID NO: 9; and/or (b) a light chain comprising the amino acid sequence of SEQ ID NO: 10. In some aspects, the anti-TL1A antibody comprises (a) a heavy chain comprising the amino acid sequence of SEQ ID NO: 9; and (b) a light chain comprising the amino acid sequence of SEQ ID NO: 10.

In some aspects of the disclosure, the anti-TL1A antibody comprises a heavy chain having the sequence shown in SEQ ID NO: 11. In some embodiments, the anti-TL1A antibody comprises a heavy chain having the sequence shown in SEQ ID NO: 11 and a light chain having the sequence shown in SEQ ID NO: 10, wherein the C-terminal lysine (K) of the heavy chain amino acid sequence of SEQ ID NO: 11 is optional. In some embodiments, the heavy chain does not have the C-terminal lysine (K). In some embodiments, the heavy chain has the sequence shown in SEQ ID NO: 9. In some embodiments, the anti-TL1A antibody comprises a heavy chain having the sequence shown in SEQ ID NO: 9 and a light chain having the sequence shown in SEQ ID NO: 10.

In some aspects of the disclosure, the anti-TL1A antibody is afimkibart (also known as RO7790121, RVT-3101, or PF-06480605).

Further exemplary anti-TL1A antibodies are set forth in Table 3 and below.

TABLE 3
Further Sequences of Exemplary Antibodies of the Disclosure

| SEQ ID | | |
|---|---|---|
| number | Description | Amino Acid Sequence |
| 22 | 1D1-1.27_VH | QVQLVQSGAEVKKPGASVKVSCKASGYDFTYYGISWVRQAPG |
| | | QGLEWMGWISTYNGNTHYARMLQGRVTMTTDTSTRTAYMELR |
| | | SLRSDDTAVYYCARENYYGSGSYRGGMDVWGQGTTVTVSS |
| 23 | 1D1-1.28_VH | QVQLVQSGAEVKKPGASVKVSCKASGYDFTYYGISWVRQAPG |
| | | QGLEWMGWISTYNGNKHYARMLQGRVTMTTDTSTRTAYMELR |
| | | SLRSDDTAVYYCARENYYGSGSYRGGMDVWGQGTTVTVSS |
| 24 | 1D1-1.29_VH | QVQLVQSGAEVKKPGASVKVSCKASGYDFTYYGISWVRQAPG |
| | | QGLEWMGWISTYNGGTHYARMLQGRVTMTTDTSTRTAYMELR |
| | | SLRSDDTAVYYCARENYYGSGSYRGGMDVWGQGTTVTVSS |
| 25 | 1D1-1.30_VH | QVQLVQSGAEVKKPGASVKVSCKASGYDFTYYGISWVRQAPG |
| | | QGLEWMGWISTYNGVTHYARMLQGRVTMTTDTSTRTAYMELR |
| | | SLRSDDTAVYYCARENYYGSGSYRGGMDVWGQGTTVTVSS |
| 26 | 1D1-1.32_VH | QVQLVQSGAEVKKPGASVKVSCKASGYDFTYYGISWVRQAPG |
| | | QGLEWMGWISTYNGGTHYARMLQGRVTMTTDTSTRTAYMELR |
| | | SLRSDDTAVYYCARENYYGSGAYRGGMDAWGQGTTVTVSS |
| 27 | 1D1-1.33_VH | QVQLVQSGAEVKKPGASVKVSCKASGYDFTYYGISWVRQAPG |
| | | QGLEWMGWISTYNGVTHYARMLQGRVTMTTDTSTRTAYMELR |
| | | SLRSDDTAVYYCARENYYGSGAYRGGMDAWGQGTTVTVSS |
| 28 | 1D1-1.34_VH | QVQLVQSGAEVKKPGASVKVSCKASGYDFTYYGISWVRQAPG |
| | | QGLEWMGWISTYNGKTHYARMHQGRVTMTTDTSTRTAYMELR |
| | | SLRSDDTAVYYCARENYYGSGAYRGGMDAWGQGTTVTVSS |
| 29 | 15A9_VH | QVQLVQSGAEVKKPGASLKVSCKASGYPFTNYGISWVRQAPGQ |
| | | GLEWMGWISTYNGNTHYAQKLQGRVTMTTDTSTTTAYMDLRSL |
| | | RSDDTAVYYCARENYYGSGSYRGGMDVWGQGTTVTVSS |
| 30 | 15C11_VH | QVQLVQSGAEVKKPGASVKVSCKASGYSFTTYGISWVRQAPGQ |
| | | GLEWMGWISTYNGNTHYAQKLQGRVTMTTDTSTRTAYMELRSL |
| | | RSDDTAVYYCARENYYGSGSYRGGMDVWGQGTTVTVSS |
| 31 | 7D4_VH | QVQLVQSGAEVKKPGASVKVSCKASGYTFTSYGINWVRQAPG |
| | | QGLEWMGWISTYNGNTNSAQKLQGRVTMTTDTSTSTAYMELR |
| | | SLRSDDTAVYYCARAHSSSWFDAFDIWGQGTMVTVSS |
| 32 | 26B11_VH | QVQLVESGGGVVQPGRSLRLSCAASGFTFSSFAMHWVRQAPG |
| | | KGLEWVALIPFDGSSNYYADSVKGRFTISRDNSKNTLYLQMNSL |
| | | RAEDTAVYYCARDRNYYGSGSFSFDAFDIWGQGTLVTVSS |
| 33 | 9B3_VH | QVQLVESGGGVVQPGRSLRLSCAASGFTFSNYALHWVRQAPG |
| | | KGLEWVALISYDGSDKYYADSVKGRFAISRDNSKNTLYLQMNSL |
| | | RAEDTAVYYCARDREYCTYSSCSYDAFDIWGQGTMVTVSS |
| 34 | 22F9_VH | QVQLVQSGAEVKKPGASVKVSCKASGYTFTSYAMHWVRQAPG |
| | | QRLEWMGWINAGNGNTKYSQKFQGRVTITRDTSASTAYMELSS |
| | | LRSEDTAVYYCARGYSSAWFDAFDIWGQGTMVTVSS |
| 35 | 7D4_VL | AIQLTQSPSSLSASVGDRVTITCRASQGISSALAWYQQKPGKAP |
| | | KLLIYDASSLESGVPSRFSGSGSGTDFTLTISSLQPEDFATYYCQ |
| | | QFNSYPLTFGGGTKVEIK |
| 36 | 26B11_VL | DIQMTQSPSSLSASVGDRVTITCRASQGISNWLAWYQQKPEKA |
| | | PKSLIYAASSLQSGVPSRFSGSGSGTDFTLTISSLQPEDFATYYC |
| | | QQYNSYPYTFGQGTKLEIK |
| 37 | 9B3_VL | DIQMTQSPSSLSASVGDRVTITCRASQGISSWLAWYQQKPEKA |
| | | PKSLIYAASSLQSGVPSRFSGSGSGTDFTLTISSLQPEDYATYYC |
| | | QQYNSYPYTFGQGTKLEIK |
| 38 | 22F9_VL | AIQLTQSPSSLSASVGDRVTITCRASQGISSALAWYQQKPGKAP |
| | | KLLIYDASSLESGVPSRFSGSGSGTDFTLTISSLQPEDFATYYCQ |
| | | QFNSYPLTFGGGTKVEIK |
| 39 | SEQ ID NO: 18 | EVQLLESGGGLVQPGKSLRLSCAVSGFTFSTYGMNWVRQAPG |
| | of WO | KGLEWVSSISGTGRTTYHADSVQGRFTVSRDNSKNILYLQMNSL |
| | 2012/064682 | RADDTAVYFCTKERGDYYYGVFDYWGQGTLVTVSS |
| 40 | SEQ ID NO: 26 | DIQMTQSPSTLSASVGDRVTITCRASQTISSWLAWYQQTPEKAP |
| | of WO | KLLIYAASNLQSGVPSRFSGSGSGTEFTLTISSLQPDDFATYYCQ |
| | 2012/064682 | QYHRSWTFGQGTKVEIT |
| 41 | C320-168 | QVQLVQSGAEVKKPGASVKVSCKASGYTFTSYDINWVRQAPGQ |
| | huIgG1-EFN of | GLEWMGWLNPNSGNTGYAQKFQGRVTMTRNTSISTAYMELSS |
| | WO | LRSEDTAVYYCAREVPETAAFEYWGQGTLVTVSSASTKGPSVF |
| | 2013/044298 | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPEAAGAPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPGK |
| 42 | C320-168 | QSVLTQPPSVSGAPGQRVTISCTSSSSDIGAGLGVHWYQQLPG |
| | huLambda of | TAPKLLIYGYYNRPSGVPDRFSGSKSGTSASLTITGLLPEDEGDY |
| | WO | YCQSYDGTLSALFGGGTKLTVLGQPKAAPSVTLFPPSSEELQAN |
| | 2013/044298 | KATLVCLISDFYPGAVTVAWKADSSPVKAGVETTTPSKQSNNKY |
| | | AASSYLSLTPEQWKSHRSYSCQVTHEGSTVEKTVAPTECS |
| 43 | VH C320-179 | QVQLVQSGAEVKKPGASVKVSCKASGYTFTSYDINWVRQAPGQ |
| | of WO | GLEWMGWLNPNSGNTGYAQKFQGRVTMTADRSTSTAYMELSS |
| | 2013/044298 | LRSEDTAVYYCAREVPETAAFEYWGQGTLVTVSS |
| 44 | VL C320-179 of | QSVLTQPPSVSGAPGQRVTISCTSSSSDIGAGLGVHWYQQLPG |
| | WO | TAPKLLIEGYYNRPSGVPDRFSGSKSGTSASLTITGLLPEDEGDY |
| | 2013/044298 | YCQSYDGTLSALFGGGTKLTVLG |
| 45 | Tulisokibart HC | QVQLVQSGAEVKKPGASVKVSCKASGFDIQDTYMHWVKQRPG |
| | | QGLEWMGRIDPASGHTKYDPKFQVRVTITRDTSTSTVYLELSSL |
| | | RSEDTAVYYCARSGGLPDVWGQGTTVTVSSASTKGPSVFPLAP |
| | | SSKSTSGGTAALGCLVKDYFPEPVTVSWNSGALTSGVHTFPAV |
| | | LQSSGLYSLSSVVTVPSSSLGTQTYICNVNHKPSNTKVDKKVEP |
| | | KSCDKTHTCPPCPAPEAAGGPSVFLFPPKPKDTLMISRTPEVTC |
| | | VVVDVSHEDPEVKFNWYVDGVEVHNAKTKPREEQYNSTYRVV |
| | | SVLTVLHQDWLNGKEYKCKVSNKALAAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPGK |
| 46 | Tulisokibart | QVQLVQSGAEVKKPGASVKVSCKASGFDIQ |
| | HFR1 | |
| 47 | Tulisokibart | DTYMH |
| | CDR-H1 | |
| 48 | Tulisokibart | WVKQRPGQGLEWMG |
| | HFR2 | |
| 49 | Tulisokibart | RIDPASGHTKYDPKFQV |
| | CDR-H2 | |
| 50 | Tulisokibart | RVTITRDTSTSTVYLELSSLRSEDTAVYYCAR |
| | HFR3 | |
| 51 | Tulisokibart | SGGLPDV |
| | CDR-H3 | |
| 52 | Tulisokibart | WGQGTTVTVSS |
| | HFR4 | |
| 53 | Tulisokibart HC | ASTKGPSVFPLAPSSKSTSGGTAALGCLVKDYFPEPVTVSWNS |
| | Tail | GALTSGVHTFPAVLQSSGLYSLSSVVTVPSSSLGTQTYICNVNH |
| | | KPSNTKVDKKVEPKSCDKTHTCPPCPAPEAAGGPSVFLFPPKP |
| | | KDTLMISRTPEVTCVVVDVSHEDPEVKFNWYVDGVEVHNAKTK |
| | | PREEQYNSTYRVVSVLTVLHQDWLNGKEYKCKVSNKALAAPIEK |
| | | TISKAKGQPREPQVYTLPPSRDELTKNQVSLTCLVKGFYPSDIAV |
| | | EWESNGQPENNYKTTPPVLDSDGSFFLYSKLTVDKSRWQQGN |
| | | VFSCSVMHEALHNHYTQKSLSLSPGK |
| 54 | Tulisokibart LC | EIVLTQSPGTLSLSPGERATLSCRASSSVSYMYWYQQKPGQAP |
| | | RPLIYATSNLASGIPDRFSGSGSGTDFTLTISRLEPEDFAVYYCQ |
| | | QWEGNPRTFGGGTKLEIKRTVAAPSVFIFPPSDEQLKSGTASVV |
| | | CLLNNFYPREAKVQWKVDNALQSGNSQESVTEQDSKDSTYSLS |
| | | STLTLSKADYEKHKVYACEVTHQGLSSPVTKSFNRGEC |
| 55 | Tulisokibart | EIVLTQSPGTLSLSPGERATLSC |
| | LFR1 | |
| 56 | Tulisokibart | RASSSVSYMY |
| | CDR-L1 | |
| 57 | Tulisokibart | WYQQKPGQAPRPLIY |
| | LFR2 | |
| 58 | Tulisokibart | ATSNLAS |
| | CDR-L2 | |
| 59 | Tulisokibart | GIPDRFSGSGSGTDFTLTISRLEPEDFAVYYC |
| | LFR3 | |
| 60 | Tulisokibart | QQWEGNPRT |
| | CDR-L3 | |
| 61 | Tulisokibart | FGGGTKLEIKRTV |
| | LFR4 | |
| 62 | Tulisokibart LC | AAPSVFIFPPSDEQLKSGTASVVCLLNNFYPREAKVQWKVDNAL |
| | Tail | QSGNSQESVTEQDSKDSTYSLSSTLTLSKADYEKHKVYACEVT |
| | | HQGLSSPVTKSFNRGEC |
| 63 | Tulisokibart VH | QVQLVQSGAEVKKPGASVKVSCKASGFDIQDTYMHWVKQRPG |
| | | QGLEWMGRIDPASGHTKYDPKFQVRVTITRDTSTSTVYLELSSL |
| | | RSEDTAVYYCARSGGLPDVWGQGTTVTVSS |
| 64 | Tulisokibart VL | EIVLTQSPGTLSLSPGERATLSCRASSSVSYMYWYQQKPGQAP |
| | | RPLIYATSNLASGIPDRFSGSGSGTDFTLTISRLEPEDFAVYYCQ |
| | | QWEGNPRTFGGGTKLEIKRTV |
| 65 | SEQ ID NO: 15 | GYTFTSYDIN |
| | of U.S. Pat. | |
| | No. 10,138,296 | |
| | (CDR-H1 of | |
| | variant 320- | |
| | 587) | |
| 66 | SEQ ID NO: 21 | WLNPNSGYTG |
| | of U.S. Pat. | |
| | No. 10,138,296 | |
| | (CDR-H2 of | |
| | variant 320- | |
| | 587) | |
| 67 | SEQ ID NO: 17 | EVPETAAFEY |
| | of U.S. Pat. | |
| | No. 10,138,296 | |
| | (CDR-H3 of | |
| | variant 320- | |
| | 587) | |
| 68 | SEQ ID NO: 18 | TSSSSDIGAGLGVH |
| | of U.S. Pat. | |
| | No. 10,138,296 | |
| | (CDR-L1 of | |
| | variant 320- | |
| | 587) | |
| 69 | SEQ ID NO: 19 | GYYNRPS |
| | of U.S. Pat. | |
| | No. 10,138,296 | |
| | (CDR-L2 of | |
| | variant 320- | |
| | 587) | |
| 70 | SEQ ID NO: 22 | QSWDGTLSAL |
| | of U.S. Pat. | |
| | No. 10,138,296 | |
| | (CDR-L3 of | |
| | variant 320- | |
| | 587) | |
| 71 | SEQ ID NO: 3 | QVQLVQSGAEVKKPGASVKVSCKASGYTFTSYDINWVRQAPGQ |
| | of U.S. Pat. | GLEWMGWLNPNSGYTGYAQKFQGRVTMTADRSTSTAYMELSS |
| | No. 10,138,296 | LRSEDTAVYYCAREVPETAAFEYWGQGTLVTVSS |
| | (VH of variant | |
| | 320-587) | |
| 72 | SEQ ID NO: 4 | QSVLTQPPSVSGAPGQRVTISCTSSSSDIGAGLGVHWYQQLPG |
| | of U.S. Pat. | TAPKLLIEGYYNRPSGVPDRFSGSKSGTSASLTITGLLPEDEGDY |
| | No. 10,138,296 | YCQSWDGTLSALFGGGTKLTVLG |
| | (VL of variant | |
| | 320-587) | |
| 73 | SEQ ID NO: 60 | QVQLVQSGAEVKKPGASVKVSCKASGYTFTSYDINWVRQAPGQ |
| | of U.S. Pat. | GLEWMGWLNPNSGYTGYAQKFQGRVTMTADRSTSTAYMELSS |
| | No. 10,138,296 | LRSEDTAVYYCAREVPETAAFEYWGQGTLVTVSSASTKGPSVF |
| | (HC) | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | KVEPKSCDKTHTCPPCPAPELLGGPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 74 | SEQ ID NO: 61 | QSVLTQPPSVSGAPGQRVTISCTSSSSDIGAGLGVHWYQQLPG |
| | of U.S. Pat. | TAPKLLIEGYYNRPSGVPDRFSGSKSGTSASLTITGLLPEDEGDY |
| | No. 10,138,296 | YCQSWDGTLSALFGGGTKLTVLGQPKAAPSVTLFPPSSEELQA |
| | (LC) | NKATLVCLISDFYPGAVTVAWKADSSPVKAGVETTTPSKQSNNK |
| | | YAASSYLSLTPEQWKSHRSYSCQVTHEGSTVEKTVAPTECS |
| 75 | SEQ ID NOS: | QVQLVQSGAEVKKPGASVKVSCKASGYTFTSYDINWVRQAPGQ |
| | 60 and 62 of | GLEWMGWLNPNSGYTGYAQKFQGRVTMTADRSTSTAYMELSS |
| | U.S. Pat. No. | LRSEDTAVYYCAREVPETAAFEYWGQGTLVTVSSASTKGPSVF |
| | 10,138,296 | PLAPSSKSTSGGTAALGCLVKDYFPEPVTVSWNSGALTSGVHT |
| | (HC with | FPAVLQSSGLYSLSSVVTVPSSSLGTQTYICNVNHKPSNTKVDK |
| | L234A/L235A/ | KVEPKSCDKTHTCPPCPAPEAAGAPSVFLFPPKPKDTLMISRTP |
| | G237A) | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMHE |
| | | ALHNHYTQKSLSLSPG |
| 76 | SEQ ID NOS: | QVQLVQSGAEVKKPGASVKVSCKASGYTFTSYDINWVRQAPGQ |
| | 60 and 64 of | GLEWMGWLNPNSGYTGYAQKFQGRVTMTADRSTSTAYMELSS |
| | U.S. Pat. No. | LRSEDTAVYYCAREVPETAAFEYWGQGTLVTVSSASTKGPSVF |
| | 10,138,296 | PLAPSSKSTSGGTAALGCLVKDYFPEPVTVSWNSGALTSGVHT |
| | (HC with YTE) | FPAVLQSSGLYSLSSVVTVPSSSLGTQTYICNVNHKPSNTKVDK |
| | | KVEPKSCDKTHTCPPCPAPELLGGPSVFLFPPKPKDTLYITREPE |
| | | VTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 77 | SEQ ID NOS: | QVQLVQSGAEVKKPGASVKVSCKASGYTFTSYDINWVRQAPGQ |
| | 60 and 66 of | GLEWMGWLNPNSGYTGYAQKFQGRVTMTADRSTSTAYMELSS |
| | U.S. Pat. No. | LRSEDTAVYYCAREVPETAAFEYWGQGTLVTVSSASTKGPSVF |
| | 10,138,296 | PLAPSSKSTSGGTAALGCLVKDYFPEPVTVSWNSGALTSGVHT |
| | (HC with | FPAVLQSSGLYSLSSVVTVPSSSLGTQTYICNVNHKPSNTKVDK |
| | L235A/G237A) | KVEPKSCDKTHTCPPCPAPELAGAPSVFLFPPKPKDTLMISRTP |
| | | EVTCVVVDVSHEDPEVKFNWYVDGVEVHNAKTKPREEQYNSTY |
| | | RVVSVLTVLHQDWLNGKEYKCKVSNKALPAPIEKTISKAKGQPR |
| | | EPQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |

In some aspects of the disclosure, the anti-TL1A antibody comprises a VH encoded by the nucleic acid sequence of the insert of the vector deposited as 1 D1 1.31 VH having ATCC accession number PTA-120639 and a VL encoded by the nucleic acid sequence of the insert of the vector deposited as 1 D1 1.31 VL having ATCC accession number PTA-120640.

In some aspects of the disclosure, the anti-TL1A antibody competes for binding with an anti-TL1A antibody comprising a variable heavy chain region having the sequence shown in SEQ ID NO: 1 and a variable light chain region having the sequence shown in SEQ ID NO: 2.

In some aspects of the disclosure, the anti-TL1A antibody competes for binding with an antibody comprising a VH encoded by the nucleic acid sequence of the insert of the vector deposited as 1D1 1.31 VH having ATCC accession number PTA-120639 and a VL encoded by the nucleic acid sequence of the insert of the vector deposited as 1D1 1.31 VL having ATCC accession number PTA-120640.

In some aspects of the disclosure, the anti-TL1A antibody comprises sequence pairs selected from the group consisting of SEQ ID NOs: 2 and 22; SEQ ID NOs: 2 and 23; SEQ ID NOs: 2 and 24; SEQ ID NOs: 2 and 25; SEQ ID NOs: 2 and 26; SEQ ID NOs: 2 and 27; SEQ ID NOs: 2 and 28; SEQ ID NOS: 2 and 29; SEQ ID NOs: 2 and 30; SEQ ID NOs: 31 and 35; SEQ ID NOs: 32 and 36; SEQ ID NOs: 33 and 37; SEQ ID NOs: 34 and 38; SEQ ID NOs: 39 and 40; SEQ ID NOs: 41 and 42; SEQ ID NOs: 43 and 44; SEQ ID NOs: 45 and 54; SEQ ID NOs: 63 and 64; SEQ ID NOs: 71 and 72; SEQ ID NOs: 73 and 74; SEQ ID NOs: 75 and 74; SEQ ID NOs: 76 and 74; and SEQ ID NOs: 77 and 74.

In some aspects of the disclosure, the anti-TL1A antibody comprises a CDR-H1 having the sequence shown in SEQ ID NO: 47, a CDR-H2 having the sequence shown in SEQ ID NO: 49, a CDR-H3 having the sequence shown in SEQ ID NO: 51, a CDR-L1 having the sequence shown in SEQ ID NO: 56, a CDR-L2 having the sequence shown in SEQ ID NO: 58, and a CDR-L3 having the sequence shown in SEQ ID NO: 60.

In some aspects of the disclosure, the anti-TL1A antibody comprises heavy chain framework regions as shown in SEQ ID NOs: 46, 48, 50, and 52 and/or comprises light chain framework regions as shown in SEQ ID NOs: 55, 57, 59, and 61.

In some aspects of the disclosure, the anti-TL1A antibody comprises a heavy chain variable region having the sequence shown in SEQ ID NO: 63 and a light chain variable region having the sequence shown in SEQ ID NO: 64.

In some aspects of the disclosure, the anti-TL1A antibody comprises a heavy chain tail sequence as provided in SEQ ID NO: 53. In some aspects of the disclosure, the anti-TL1A antibody comprises a light chain tail sequence as provided in SEQ ID NO: 62.

In some aspects of the disclosure, the anti-TL1A antibody comprises a heavy chain having the sequence shown in SEQ ID NO: 45 and/or a light chain having the sequence shown in SEQ ID NO: 54.

In some aspects of the disclosure, the anti-TL1A antibody comprises a heavy chain having the sequence shown in SEQ ID NO: 45 and a light chain having the sequence shown in SEQ ID NO: 54.

In some aspects of the disclosure, the anti-TL1A antibody is tulisokibart.

In some aspects, the antibody used in any of the methods, compositions, uses, and compositions for use provided herein is an anti-TL1A antibody provided in Table 2A of U.S. Pat. No. 11,136,386, which is incorporated herein by reference in its entirety.

In some aspects of any of the methods, compositions, uses, and compositions for use provided herein, the anti-TL1A antibody is an anti-TL1A antibody provided in U.S. Pat. Nos. 10,322,174, 10,689,439, 11,292,848, 10,138,296, 10,822,422, and 11,220,549, which are incorporated herein by reference in their entirety. In some embodiments, the anti-TL1A antibody comprises the CDR sequences of the 320-179 clone provided in U.S. Pat. No. 10,689,439. In some embodiments, the anti-TL1A antibody is the 320-179 clone provided in U.S. Pat. No. 10,689,439.

In some aspects of the disclosure, the anti-TL1A antibody comprises a CDR-H1 having the sequence shown in SEQ ID NO: 65, a CDR-H2 having the sequence shown in SEQ ID NO: 66, a CDR-H3 having the sequence shown in SEQ ID NO:67, a CDR-L1 having the sequence shown in SEQ ID NO: 68, a CDR-L2 having the sequence shown in SEQ ID NO: 69, and a CDR-L3 having the sequence shown in SEQ ID NO: 70.

In some aspects of the disclosure, the anti-TL1A antibody comprises a heavy chain variable region having the sequence shown in SEQ ID NO: 71 and a light chain variable region having the sequence shown in SEQ ID NO: 72.

In some aspects of the disclosure, the anti-TL1A antibody comprises a heavy chain having the sequence shown in SEQ ID NO: 73, 75, 76, or 77 and/or a light chain having the sequence shown in SEQ ID NO: 74. In some aspects of the disclosure, the anti-TL1A antibody comprises a heavy chain having the sequence shown in SEQ ID NO: 73, 75, 76, or 77 and a light chain having the sequence shown in SEQ ID NO: 74. In some aspects of the disclosure, the anti-TL1A antibody comprises a heavy chain having the sequence shown in SEQ ID NO: 73 and a light chain having the sequence shown in SEQ ID NO: 74. In some aspects of the disclosure, the anti-TL1A antibody comprises a heavy chain having the sequence shown in SEQ ID NO: 75 and a light chain having the sequence shown in SEQ ID NO: 74. In some aspects of the disclosure, the anti-TL1A antibody comprises a heavy chain having the sequence shown in SEQ ID NO: 76 and a light chain having the sequence shown in SEQ ID NO: 74. In some aspects of the disclosure, the anti-TL1A antibody comprises a heavy chain having the sequence shown in SEQ ID NO: 77 and a light chain having the sequence shown in SEQ ID NO: 74.

In some aspects of the disclosure, the anti-TL1A antibody is TEV-48574.

In some aspects of the disclosure, the anti-TL1A antibody is C03V. C03V has been described, for example, in Clarke A W, Poulton L, Shim D, Mabon D, Butt D, Pollard M, Pande V, Husten J, Lyons J, Tian C, Doyle A G. An anti-TL1A antibody for the treatment of asthma and inflammatory bowel disease. MAbs. 2018 May/June; 10 (4): 664-677. doi: 10.1080/19420862.2018.1440164. Epub 2018 Mar. 5. PMID: 29436901; PMCID: PMC5973687.

In some aspects of the disclosure, the anti-TL1A antibody is SPY002. SPY002 has been described, for example, in Zhu, E., et al. “P911 Development and Characterization of SPY002, a Novel Extended Half-life Monoclonal Antibody Drug Candidate Targeting TL1A for the Treatment of IBD.” Journal of Crohn's and Colitis 18.Supplement_1 (2024): i1666-i1666.

In some aspects of the disclosure, the anti-TL1A antibody is FG-M701.

### IV. Articles of Manufacture and Kits

In another aspect of the invention, an article of manufacture containing materials useful for the treatment, prevention and/or diagnosis of the disorders described above (e.g., inflammatory bowel disease (IBD), e.g., ulcerative colitis (UC) or Crohn's disease (CD)) is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition (e.g., a composition comprising an anti-TL1A antibody) which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention (e.g., an anti-TL1A antibody, e.g., afimkibart). The label or package insert indicates that the composition is used for treating the condition of choice (e.g., IBD, e.g., UC or CD). Moreover, the article of manufacture may comprise (a) a first container with a composition contained therein, wherein the composition comprises an antibody of the invention; and (b) a second container with a composition contained therein, wherein the composition comprises a further therapeutic agent. The article of manufacture in this aspect of the invention may further comprise a package insert indicating that the compositions can be used to treat a particular condition (e.g., IBD, e.g., UC or CD). Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The disclosure also provides kits comprising any or all of the anti-TL1A antibodies described herein. Kits of the disclosure include one or more containers comprising an anti-TL1A antibody described herein and instructions for use in accordance with any of the methods of the disclosure described herein. Generally, these instructions comprise a description of administration of the anti-TL1A antibody for the above-described therapeutic treatments. In some aspects, kits are provided for producing a single-dose administration unit. In certain aspects, the kit can contain both a first container having a dried protein and a second container having an aqueous formulation. In certain aspects, kits containing single and multi-chambered pre-filled syringes (e.g., liquid syringes and lyosyringes) are included.

The instructions relating to the use of an anti-TL1A antibody generally include information as to dosage, dosing schedule, and route of administration for the intended treatment. The containers may be unit doses, bulk packages (e.g., multi dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-TL1A antibody. The container may further comprise a second pharmaceutically active agent.

Kits may optionally provide additional components such as buffers and interpretive information. Normally, the kit comprises a container and a label or package insert(s) on or associated with the container.

### V. Compositions and Formulations

### A. Compositions

In a further aspect, provided are pharmaceutical compositions comprising an effective amount of an anti-TL1A antibody as described herein, and such pharmaceutical compositions for use in any of the methods of treatment provided herein. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions (formulations) of an anti-TL1A antibody as described herein can be prepared by combining the antibody with pharmaceutically acceptable carriers or excipients known to the skilled person. See, for example Remington's Pharmaceutical Sciences 16th edition, Osol, A. Ed. (1980) and Falconer R. J., Biotechnology Advances 37:107412 (2019). Exemplary pharmaceutical compositions of an anti-TL1A antibody as described herein may be lyophilized, aqueous, frozen, etc.

Pharmaceutically acceptable carriers are generally non-toxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredient as necessary for the particular indication being treated, preferably those with complementary activities that do not adversely affect each other. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

The anti-TL1A antibody, and compositions thereof, can also be used in conjunction with, or administered separately, simultaneously, or sequentially with other agents that serve to enhance and/or complement the effectiveness of the agents.

### B. Formulations

Therapeutic formulations of the anti-TL1A antibody used in accordance with the present disclosure are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes that may contain the anti-TL1A antibody are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic anti-TL1A antibody compositions are generally placed into a container having a sterile access port, for example, an intravenous solution bag or vial having a stopper pierceable by a hypodermic injection needle.

The compositions according to the present disclosure may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion.

Suitable emulsions will typically contain up to 20% oil, for example, between 5 and 20%. The fat emulsion can comprise fat droplets between 0.1 and 1.0 pm, particularly 0.1 and 0.5 μm, and have a pH in the range of 5.5 to 8.0.

The emulsion compositions can be those prepared by mixing an anti-TL1A antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some aspects, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

In aspects that refer to a method of treating IBD as described herein, such aspects are also further aspects of an anti-TL1A antibody for use in that treatment, or alternatively of the use of an anti-TL1A antibody in the manufacture of a medicament for use in that treatment.

### VII. EXAMPLES

### Example 1. Phase III, Multicenter, Double-Blind, Placebo Controlled Studies to Assess the Efficacy and Safety of Induction Therapy with Afimkibart in Patients with Moderately to Severely Active Ulcerative Colitis

The GA45329 study provided in these Examples is a Phase III, multicenter, double-blind, placebo-controlled treat-through study designed to assess the efficacy and safety of induction and maintenance therapy with afimkibart (formerly PF 06480605, RVT-3101, or RO7790121) in patients with moderately to severely active ulcerative colitis (UC). Afimkibart is a fully human immunoglobulin G1 (IgG1) monoclonal antibody (mAb) against tumor necrosis factor-like ligand 1A (TL1A). TL1A plays a central role in the regulation of gut mucosal immunity and participates in immunological and fibrosis pathways involved in inflammatory bowel disease (IBD) pathogenesis by binding its receptor, death receptor 3 (DR3) (Shih et al., Clin Pharmacol Drug Dev, 7:1492-1503, 2014; Xu and Huang, Front Immunol, 13:891328, 2022). Binding of afimkibart to TL1A prevents the binding and signaling of TL1A to death receptor 3 on immune cells, leading to anti-inflammatory and anti-fibrotic effects.

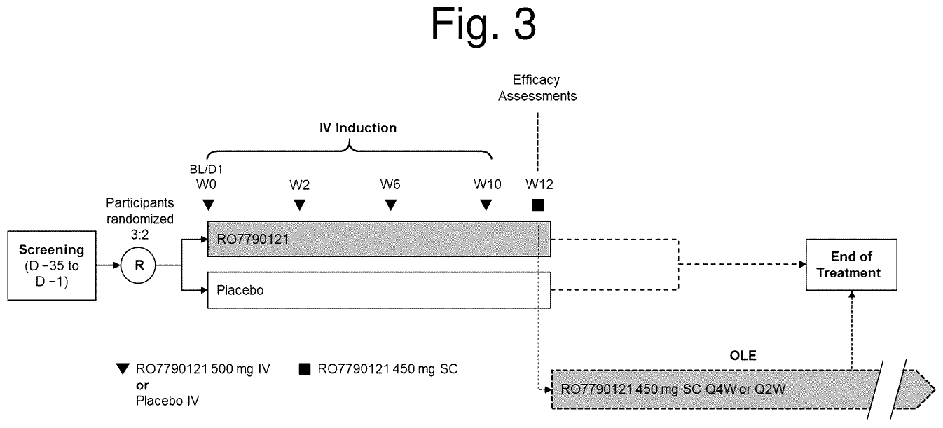

The GA45330 study provided in these Examples is a Phase 3, multicenter, double-blind, placebo-controlled study designed to assess the efficacy, safety, pharmacokinetics, and pharmacodynamics of induction therapy with afimkibart compared with placebo in patients with moderately to severely active UC. A study schema for the GA45330 study is provided in FIG. 3.

Therapeutic options have expanded substantially over the past decade, with biologics (e.g., anti-tumor necrosis factor (TNF), anti-IL-12/23, and anti-integrin molecule mAbs) and small molecule treatments (e.g., Janus kinase (JAK) inhibitors and sphingosine 1 phosphate (S1P) receptor modulators) now available in addition to conventional therapies. However, a high unmet medical need remains for treatments with better benefit-risk profiles that attenuate inflammation and clinical sequelae and provide sustained control to improve the long-term prognosis of patients with UC.

### A. Co-Primary Objectives and Corresponding Estimands

The co-primary objectives of the GA45329 trial are to evaluate the efficacy of afimkibart in inducing and maintaining clinical remission compared with placebo (Table 4).

The primary objective of the GA45330 trial is to evaluate the efficacy of afimkibart in inducing clinical remission compared with placebo (Table 5).

Clinical remission is defined on the basis of the modified Mayo Score (mMS), described in Example 5. Statistical inference supporting this evaluation targets estimands representing the effect of assignment to the treatment conditions (afimkibart vs. placebo; Example 4) on a specified outcome (endpoint) in the population of patients with moderately to severely active UC, as identified by key trial inclusion and exclusion criteria (Example 3).

TABLE 4
Co-Primary Objectives and Endpoints of the GA45329 Trial

| Co-Primary Objectives | Endpoints |
|---|---|
| To evaluate the efficacy | Clinical remission, defined as modified Mayo |
| of afimkibart compared | Score (mMS) ≤2 with stool frequency subscore |
| with placebo in inducing | (SFS) = 0 or 1, rectal bleeding subscore |
| remission. | (RBS) = 0, and endoscopic subscore (ES) = |
| | 0 or 1, at Week 12. |
| To evaluate the efficacy | Clinical remission, as defined above, at Week |
| of afimkibart compared | 52. |
| with placebo in | |
| maintaining remission. | |

TABLE 5
Primary Objective and Endpoint of the GA45330 Trial

| Primary Objective | Endpoints |
|---|---|
| To evaluate the efficacy of | Clinical remission, defined as mMS ≤2 |
| afimkibart compared with | with SFS = 0 or 1, RBS = 0, and |
| placebo in inducing remission. | ES = 0 or 1, at Week 12. |

As noted by the International Council for Harmonisation (ICH) E9 (R1) addendum on estimands and sensitivity analysis in clinical trials (ICH 2020) to the guideline on statistical principles for clinical trials, the availability or interpretation of endpoint measurements may be affected by the occurrence of intercurrent events (ICEs; ICH 2020) arising between randomization and endpoint assessment. Strategies to address anticipated ICEs are summarized in Table 6.

TABLE 6
Strategies for Anticipated Intercurrent Events

| Anticipated Intercurrent Event (ICE) | Strategy |
|---|---|
| Treatment discontinuation | Composite: ICE is considered indicative of |
| Increase from baseline or initiation of | treatment failure, and the affected endpoint |
| permitted or prohibited concomitant | measurement will be imputed to a value that is |
| medications to treat UC (Example 3), due | deemed unfavorable. |
| to lack of efficacy. | For the primary or co-primary endpoints, this value |
| UC-related surgery. | corresponds to not achieving clinical remission. |
| Decrease in permitted concomitant | Treatment policy: Ignore ICE in statistical inference. |
| medications for UC. | |

Under these strategies, the estimand supporting the first co-primary objective of the GA45329 trial and the primary objective of the GA45330 trial amounts to a treatment effect summarized by the difference between two proportions:
the percentage of patients from the target population in clinical remission upon a successful course of induction therapy, after assignment to afimkibart; versus
this same percentage, had these patients been instead assigned to placebo.

In the GA45329 trial, the estimand for the co-primary objective in maintenance is defined similarly.

Primary analysis of the GA45329 study data will yield estimates for these induction and maintenance treatment effects. Should both estimates favor afimkibart over placebo and be deemed statistically significant, the study will be considered positive.

Primary analysis of the GA45330 study data will yield an estimate for the induction treatment effect. Should the estimate favor afimkibart over placebo and be deemed statistically significant, the study will be considered positive.

Details on statistical hypothesis testing are described in Example 6.

### B. Secondary Objectives and Corresponding Endpoints

Efficacy is further considered in secondary objectives, described below (see Tables 7-10). As with the primary or co-primary objectives, the secondary efficacy objectives have corresponding estimands of interest representing an effect comparing the same treatment conditions (afimkibart vs. placebo; Example 4) on a specified endpoint, within the same overall population of patients (Example 3), and using the same strategies for anticipated intercurrent events (Table 6). These treatment effects are defined in the same manner as those for the primary or co-primary endpoints, with effects on binary efficacy endpoints summarized by an afimkibart versus placebo difference between proportions. For other efficacy endpoints, the effect is generally a difference in a summary outcome measure, such as a mean score or mean change from baseline score. Further details on the assessment and evaluation of different efficacy endpoint types are provided in Examples 5 and 6, respectively.

TABLE 7
Secondary Objectives and Endpoints of the GA45329 trial

| Key Secondary Objectives | Corresponding Endpoints |
|---|---|
| To evaluate the efficacy of | Partial modified Mayo Score (pmMS), defined as stool frequency |
| afimkibart compared with | subscore (SFS) + rectal bleeding subscore (RBS), from baseline |
| placebo in inducing | to Week 2. |
| response. | Endoscopic improvement, defined as endoscopic subscore (ES) = 0 |
| | or 1, at Week 12. |
| | Endoscopic remission, defined as ES = 0, at Week 12. |
| | Clinical response, defined as a decrease in modified Mayo Score; |
| | (mMS) of at least 2 points and 30% from baseline and either a |
| | decrease in RBS ≥1 or RBS = 0 or 1, at Week 12. |
| | Histologic improvement, defined as Geboes Grading Scale score |
| | (Geboes) ≤3.1 at Week 12. |
| | Histologic remission, defined as Geboes <2B at Week 12. |
| | Histologic-endoscopic mucosal improvement, defined as |
| | Geboes ≤3.1 and ES = 0 or 1, at Week 12. |
| | Histologic-endoscopic remission, defined as Geboes <2B and |
| | ES = 0 or 1, at Week 12. |
| To evaluate the efficacy of | Maintenance of remission, defined as clinical remission at both |
| afimkibart compared with | Weeks 12 and 52. |
| placebo in maintaining | Corticosteroid-free remission, defined as clinical remission at |
| response. | Week 52 and no use of corticosteroids for UC at least 8 weeks |
| | prior to Week 52. |
| | Endoscopic improvement, as defined above, at Week 52. |
| | Endoscopic remission, as defined above, at Week 52. |
| | Histologic improvement, as defined above, at Week 52. |
| | Histologic remission, as defined above, at Week 52. |
| | Histologic-endoscopic mucosal improvement, as defined above, at |
| | Week 52. |
| | Histologic-endoscopic remission, as defined above, at Week 52. |
| To evaluate the efficacy of | Among TL1A biomarker-defined subgroups of participants: |
| afimkibart compared with | Clinical remission at Week 12. |
| placebo in TL1A biomarker- | Clinical remission at Week 52. |
| defined subpopulations of | Endoscopic improvement at Week 12. |
| participants. | Endoscopic improvement at Week 52. |
| To evaluate the efficacy of | Bowel urgency from baseline through Week 52. |
| afimkibart compared with | Abdominal pain from baseline through Week 52. |
| placebo in terms of | Fatigue, as measured by Functional Assessment of Chronic Illness |
| ulcerative colitis (UC)- | Therapy-Fatigue Scale (FACIT-F), from baseline to Week 12 |
| related symptoms and | and Week 52. |
| health-related quality of life. | Inflammatory Bowel Disease Questionnaire (IBDQ) from baseline to |
| | Week 12 and Week 52. |

TABLE 8
Secondary Objectives and Endpoints of the GA45330 trial

| Key Secondary Objectives | Corresponding Endpoints |
|---|---|
| To evaluate the efficacy of | pmMS, defined as SFS + RBS, from baseline to Week 2. |
| afimkibart compared with | Endoscopic improvement, defined as ES = 0 or 1, at Week 12. |
| placebo in inducing | Endoscopic remission, defined as ES = 0, at Week 12. |
| response. | Clinical response, defined as a decrease in mMS of at least |
| | 2 points and 30% from baseline and either a decrease in |
| | RBS ≥1 or RBS = 0 or 1, at Week 12. |
| | Histologic improvement, defined as Geboes Grading Scale score |
| | (Geboes) ≤3.1 at Week 12. |
| | Histologic remission, defined as Geboes <2B at Week 12. |
| | Histologic-endoscopic mucosal improvement, defined as |
| | Geboes ≤3.1 and ES = 0 or 1, at Week 12. |
| | Histologic-endoscopic remission, defined as Geboes <2B and |
| | ES = 0 or 1, at Week 12. |
| To evaluate the efficacy of | Among TL1A biomarker-defined subgroups of participants: |
| afimkibart compared with | Clinical remission at Week 12. |
| placebo in TL1A biomarker- | Endoscopic improvement at Week 12. |
| defined subpopulations of | |
| participants. | |
| To evaluate the efficacy of | Bowel urgency from baseline through Week 12. |
| afimkibart compared with | Abdominal pain from baseline through Week 12. |
| placebo in terms of | Fatigue, as measured by Functional Assessment of Chronic Illness |
| ulcerative colitis (UC)- | Therapy-Fatigue Scale (FACIT-F), from baseline to Week 12. |
| related symptoms and | Inflammatory Bowel Disease Questionnaire (IBDQ) from baseline to |
| health-related quality of life. | Week 12. |

The TL1A biomarker is TNFSF15 haplotype B carrier status (haplotype B carrier vs. haplotype B non-carrier). TNFSF15 haplotype B non-carrier patients may potentially obtain additional benefit from afimkibart therapy. See Section II (H), above.

Other secondary objectives of the studies (Tables 9 and 10) also consider both efficacy and safety. Safety endpoints refer to a summary outcome measure, rather than a participant-level outcome variable.

TABLE 9
Other Secondary Objectives and Endpoints of the GA45329 trial

| Other Secondary Objectives | Corresponding Endpoints |
|---|---|
| To evaluate the efficacy of | Overall change in UC symptoms, as measured by Patient Global |
| afimkibart compared with placebo | Impression of Change (PGIC), from baseline to Week 2, Week 12, |
| in terms of the participant's global | and Week 52. |
| impressions. | Overall severity in UC symptoms, as measured by Patient Global |
| | Impression of Severity (PGIS), from baseline to Week 2, Week 12, |
| | and Week 52. |
| To evaluate the safety of | Incidence and severity of the following: |
| afimkibart compared with placebo. | Adverse events. |
| | Serious adverse events. |
| | Adverse events leading to study treatment discontinuation. |
| | Adverse events of special interest. |

TABLE 10
Other Secondary Objectives and Endpoints of the GA45330 trial

| Other Secondary | |
|---|---|
| Objectives | Corresponding Endpoints |
| To evaluate the efficacy | Overall change in UC symptoms, as measured |
| of afimkibart compared | by Patient Global Impression of Change |
| with placebo in terms of | (PGIC), from baseline to Week 2 and Week 12. |
| the participant's global | Overall severity in UC symptoms, as measured |
| impressions. | by Patient Global Impression of Severity |
| | (PGIS), from baseline to Week 2 and Week 12. |
| To evaluate the safety | Incidence and severity of the following: |
| of afimkibart compared | Adverse events. |
| with placebo. | Serious adverse events. |
| | Adverse events leading to study treatment |
| | discontinuation. |
| | Adverse events of special interest. |

### C. Exploratory Objectives and Endpoints

Exploratory objectives and corresponding endpoints are described below (Table 11). Estimands for exploratory efficacy objectives are defined in a similar manner to those supporting primary and secondary objectives. As with safety domains, exploratory objectives beyond efficacy may have endpoints that describe a summary outcome measure rather than a participant-level outcome variable.

TABLE 11
Exploratory Objectives and Endpoints

| Exploratory Objectives | Corresponding Endpoints |
|---|---|
| To characterize the | Pre-dose and peak concentration of afimkibart in serum at |
| pharmacokinetics of afimkibart. | specified timepoints. |
| To evaluate the immunogenicity of | Prevalence of anti-drug antibodies (ADAs) and neutralizing |
| afimkibart. | antibodies (NAbs) at baseline and incidence of ADAs and |
| | NAbs in serum at specified timepoints. |
| | Association of ADA and NAb status with efficacy, safety, or |
| | pharmacokinetic (PK) endpoints. |
| To evaluate the pharmacodynamics | Total soluble tumor necrosis factor-like ligand 1A (TL1A) |
| of afimkibart. | concentration in serum at specified timepoints. |
| | Association of these levels with PK, immunogenicity, and |
| | disease activity. |
| To evaluate the health utility of | EuroQol 5-Dimension 5-Level (EQ-5D-5L) index-based and |
| participants treated with afimkibart | visual analog scale (VAS) scores from baseline to Week 12 |
| compared with placebo. | (GA45330 trial) or to Week 12 and Week 52 (GA45329 trial). |
| | WPAI:UC = Work Productivity and Activity Impairment |
| | Questionnaire:Ulcerative Colitis (WPAI:UC) scores from |
| | baseline to Week 12 (GA45330 trial) or to Week 12 and |
| | Week 52 (GA45329 trial). |
| To evaluate biomarkers in | Fecal calprotectin at specified timepoints. |
| participants treated with afimkibart | C-reactive protein at specified timepoints. |
| compared with placebo. | Pathway and pathophysiology biomarker levels from baseline |
| | through Week 12 (GA45330 trial) or Week 52 (GA45329 |
| | trial), and associations of these levels amongst each other |
| | and with PK, pharmacodynamics (PD), and disease activity. |
| To evaluate automated endoscopy | Artificial intelligence (AI)-based assessment of endoscopic |
| assessments in participants treated | activity from baseline to Week 12 (GA45330 trial) or Week |
| with afimkibart or placebo. | 12 and Week 52 (GA45329 trial). |
| | AI-based spatial measurements of mucosal features, from |
| | baseline to Week 12 (GA45330 trial) or Week 12 and |
| | Week 52 (GA45329 trial). |
| | Association of these assessments with treatment arm and with |
| | disease activity. |

### Example 2. UC Study Design

A. Overall design

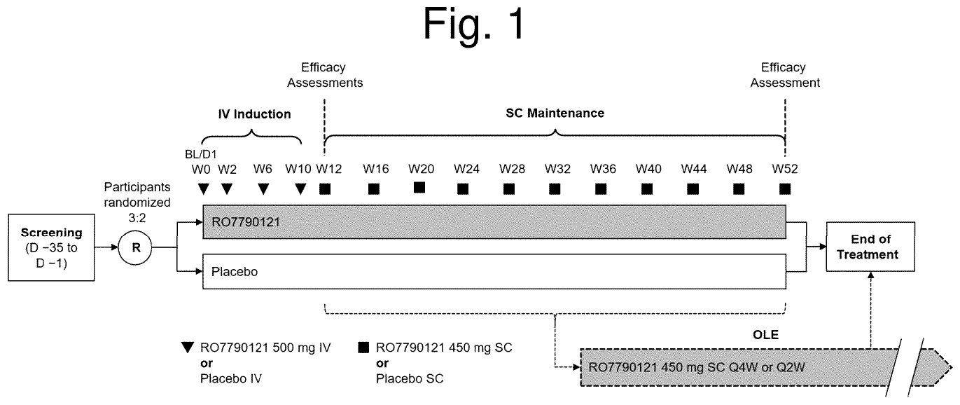

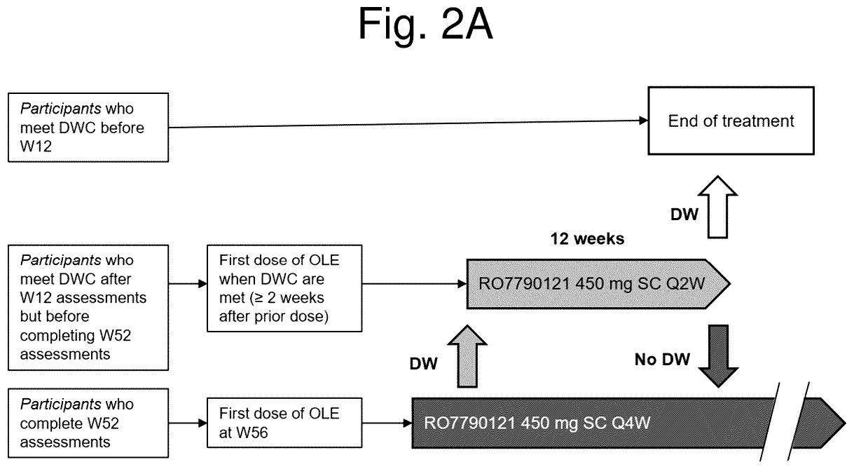

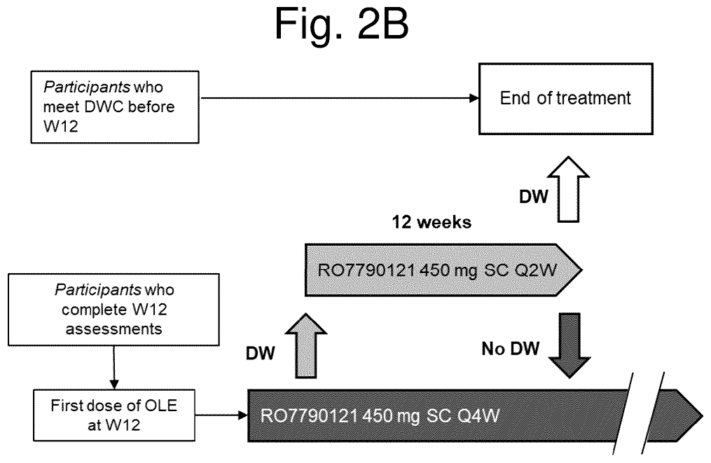

The GA45329 study is a Phase III, multicenter, randomized, double-blind, placebo-controlled, treat-through study to evaluate the efficacy and safety of afimkibart in patients with moderately to severely active UC. Study schemata for the GA45329 study are provided in FIGS. 1 and 2A. The GA45329 study includes an induction phase, a maintenance phase, and an optional open-label extension (OLE) phase. The GA45330 study (FIGS. 2B and 3) includes only the induction phase and the optional OLE phase.

The study population of both studies includes participants with moderately to severely active UC who have failed (1) prior conventional (aminosalicylates, corticosteroids and/or immunosuppressants) (termed “conventional therapy failure” in this protocol), OR (2) prior advanced therapy, which includes biologics or targeted small molecules, e.g., anti-TNF, anti-IL12/23, anti-integrin, S1P receptor modulators, JAK inhibitors, etc. (termed “advanced therapy failure” in this protocol).

Approximately 400 total participants in the GA45329 study and 350 total participants in the GA45330 study are enrolled across global investigational sites, with balanced representation of participants who have demonstrated conventional or advanced therapy failure. Enrollment of participants who have failed three or more advanced therapies (i.e., three therapies, not three classes of therapies) is capped to at most 30% of the total population.

The GA45329 study has a treat-through design that consists of a screening period of up to 35 days (+7 days) to determine eligibility; a 12-week induction treatment phase; a 40-week maintenance treatment phase; an optional open-label extension (OLE) treatment phase; and a safety follow-up period of 12 weeks (consisting of two visits, one at 6 weeks and one at 12 weeks) following the final dose of study treatment. The GA45330 omits the maintenance treatment phase. Entry criteria are based on confirmation of moderately to severely active UC during screening, defined as a modified Mayo Score (mMS) of 5 to 9, including an endoscopic subscore(ES) ≥2, as confirmed by a centrally-read endoscopy (flexible sigmoidoscopy or colonoscopy).

Eligible study participants in the GA45329 study are randomly assigned to one of the following treatment arms:
Afimkibart: 500 mg intravenously (IV) at Weeks 0, 2, 6, and 10, followed by 450 mg subcutaneously (SC) every 4 weeks (Q4W) from Week 12 through Week 52.
Placebo: placebo IV at Weeks 0, 2, 6, and 10, followed by placebo SC Q4W from Week 12 through Week 52.

Eligible study participants in the GA45330 study are randomly assigned to one of the following treatment arms:
Afimkibart: 500 mg intravenously (IV) at Weeks 0, 2, 6, and 10.
Placebo: placebo IV at Weeks 0, 2, 6, and 10.

Randomization is performed according to a 3:2 allocation ratio and stratified by: prior advanced therapy at baseline (yes/no); baseline corticosteroid use (yes/no); and disease severity (mMS of 5 to 6 and mMS of 7 to 9).

The induction phase (dosing during Weeks 0-10) evaluates the induction of clinical remission, measured at Week 12. After completion of the induction phase, participants in the GA45329 study continue with the SC administration of afimkibart or matching placebo during the maintenance phase (Weeks 12-52), where the durability of clinical response and remission are examined. From Week 12 onwards, participants who have completed Week 12 (in either the GA45329 or GA45330 study) are eligible to enter the OLE phase, provided that their condition meets the disease worsening criteria. Participants who do not complete the 12-week induction period for any reason are withdrawn from blinded treatment and proceed into the mandatory treatment discontinuation/early withdrawal visit followed by the post-treatment safety follow-up visits.

Disease worsening continues to be monitored by the investigator after Week 12. Participants who complete all trial procedures, including study treatment administration at Week 52 (in the GA45329 study), or who meet disease worsening criteria any time during the maintenance phase, have the option to continue to the OLE phase and receive active treatment. Participants in the GA45329 study who discontinue the maintenance phase before Week 52 and do not enter the OLE phase proceed to the mandatory treatment discontinuation/early withdrawal visit followed by the post-treatment safety follow-up visits.

The aim of the studies is to assess the efficacy and safety of afimkibart compared to placebo. Efficacy is assessed by primary or co-primary endpoints of clinical remission (defined based on mMS) at Week 12 (induction) (in both the GA45329 and GA45330 studies) and at Week 52 (maintenance) in the GA45329 study.

The primary or co-primary, secondary, exploratory, safety, PK, PD, immunogenicity and any other objectives and corresponding endpoints of this study are described in Example 1. Participants undergo periodic efficacy and safety assessments.

The primary or co-primary endpoints and certain secondary efficacy endpoints are based on components of the mMS: endoscopic subscore(ES), stool frequency subscore (SFS), and rectal bleeding subscore (RBS). These subscores are evaluated from centrally read endoscopic findings and participant-reported stool frequency and rectal bleeding ratings. These participant-reported UC symptoms, as well as bowel urgency and abdominal pain, are collected daily using an electronic Diary (eDiary) during the screening and induction phases. During the maintenance phase of the GA45329 study, these patient-reported outcomes (PROs) are collected by eDiary daily. During the OLE phase of either study, participants complete eDiary PROs for at least 7 days prior to each Q3M (every three months) study visit during the first year, and prior to annual study visits thereafter. If disease worsening criteria assessment is required in the OLE phase, eDiary PROs are collected for at least 7 days prior to the study visit at which the assessment is being conducted and at which dosing frequency may be adjusted.

### B. Open-Label Extension Phase

All participants, irrespective of whether they were previously randomized to receive afimkibart or placebo during the double-blind phase, have the opportunity to participate in the optional OLE phase of the study with access to afimkibart and monitoring, provided they meet specified eligibility criteria. A schema for the OLE phase of the GA45329 study is provided in FIG. 2A. A schema for the OLE phase of the GA45330 study is provided in FIG. 2B.

i. Open-Label Extension Phase Eligibility Criteria

Participants are eligible to enter the OLE once they have provided consent to participate, and, per investigator assessment, participant safety would not be at risk by continuing participation in the optional OLE phase of the study.

Participants may be eligible to enter OLE at the following time points:
At any time after completion of the 12-week induction phase and upon completion of the Week 12 assessments and first dose of maintenance, if the participant meets disease worsening criteria (below). Participants are not eligible for the OLE phase before Week 12.
In the GA45329 study, after completion of the 40-week maintenance phase and upon completion of the Week 52 assessments.
   ii. Disease Worsening Criteria

Starting after the Week 12 assessments (and the first dose of maintenance therapy in the GA45329 study), participants who, based on assessment by the investigator, have not improved or have worsened compared with baseline (Day 1 of Week 0), may be eligible to enroll in the OLE, provided they meet both of the following criteria:
ES ≥2, based on central reading.
An endoscopy assessment is required to confirm OLE eligibility for participants discontinuing the double-blind maintenance phase prior to Week 52 and should be performed upon the appearance of UC symptoms. However, the procedure need not be repeated if performed within the last 6 weeks.
Rectal bleeding rating ≥2 on at least three days within the past seven days, excluding any day coinciding with bowel preparation and endoscopy.

Fulfillment of these criteria is captured by efficacy assessments, namely the endoscopy and rectal bleeding assessments under the Mayo Score (Example 5). In the evaluation of disease worsening or potential differential diagnoses, the presence of enteric pathogens is to be ruled out by stool culture/sensitivity ova and parasite evaluation and should include C. difficile testing. If CMV infection is suspected, tissue biopsy is to be performed.

Only during the OLE phase, for participants who experience symptoms of disease worsening, the investigator may use their discretion in the need for unscheduled endoscopies to determine changes to the OLE dose schedule. Should the investigator decide to forgo such an endoscopy in the assessment of disease worsening in the OLE, it is permissible to apply the symptomatic criteria (rectal bleeding rating) in conjunction with an alternative objective criterion of fecal calprotectin ≥150 μg/g per the American Gastroenterological Association Guidelines (Singh et al., Gastroenterology, 164 (3): 344-372, 2023).

iii. Open-Label Extension Phase Dose Schedule

After Week 12 (e.g., during the maintenance phase of the GA45329 study), if the participant's condition meets the disease worsening criteria provided above, the participant may enter the OLE every 2 weeks (Q2W) dose intensification schedule (450 mg SC Q2W) for 12 weeks or be withdrawn from the study as assessed by the investigator. Participants who follow the Q2W dose schedule have the opportunity to de-escalate to the 450 mg SC Q4W dose schedule after 12 weeks or be withdrawn from the study as assessed by the investigator (FIGS. 2A and 2B).

Participants who complete the 52-week study in the GA45329 study or the 12-week study in the GA45330 study and roll over into the OLE phase continue with the Q4W dose schedule (450 mg SC Q4W). The first OLE visit is at Week 56 in the GA45329 study. Participants continue in the OLE phase on the Q4W dose regimen until the end of the study. However, if at any point during the OLE phase, the participant's condition meets the disease worsening criteria, the option for dose intensification to 450 mg SC Q2W or withdrawal from the study is assessed by the investigator. Participants who follow the 450 mg SC Q2W dose schedule may de-escalate to the 450 mg SC Q4W dose schedule after 12 weeks or be withdrawn from the study as assessed by the investigator. Doses of afimkibart should be administered at least 7 days apart. There is a maximum of 2 dose intensifications to Q2W allowed in the OLE phase of the studies.

The OLE phase dosing schedule is shown in Table 12.

TABLE 12
Open-Label Extension Phase Dosing Schedule

| | | | First Dose in the |
|---|---|---|---|
| Scenario | OLE Rollover? | OLE Dosage | OLE Phase |
| Study completion at W 52 | Yes | Q4W | W 56 (GA45329 |
| (GA45329 study) or W 12 | | | study) |
| (GA45330 study) | | | W 12 (GA45330 |
| | | | study) |
| DWC met before W 12 | No OLE rollover | NA | NA |
| | End of treatment | | |
| | Study | | |
| | discontinuation | | |
| | (Tx Disc/Early With/ | | |
| | SFU at W 6, W 12) | | |
| DWC met >W 12 to <W 52 | Yes | Q2W | When DWC is met |
| (GA45329 study) | | (dose intensification) | (≥2 weeks after |
| | | for 12 weeks then | prior dose) |
| | | de-escalate to Q4W^{a} | |
| DWC met during OLE | NA | Q2W | NA |
| | | (dose intensification) | |
| | | for 12 weeks then | |
| | | de-escalate to Q4W^{a} | |

DWC = disease worsening criteria; Early With = early withdrawal; FU = follow-up; NA = not applicable; OLE = open-label extension phase; Q2W = every 2 weeks; Q4W = every 4 weeks; SFU = safety follow-up; Tx Disc = treatment discontinuation; W = Week.
Note:
There will be a maximum of two dose intensifications (Q4W dosing→Q2W dosing) allowed in the OLE phase of the study.
^{a}Decision to de-escalate to Q4W or discontinue the participant from the study is by investigator assessment.

### C. End of Study Definition and Duration of Participation

A participant is considered to have completed the study if he or she has completed all phases of the study, including the last visit.

The end of the GA45329 or GA45330 study is defined as the date of the last visit of the last participant in the study or the date at which the last data point required for statistical analysis, safety follow-up, or statistical analysis follow-up is received from the last participant, whichever occurs later.

The end of the GA45329 study is expected to occur approximately 70 weeks after the last participant is enrolled.

In the GA45329 study, the total maximum duration of study participation for an individual is expected to be approximately 70 weeks without OLE participation. With OLE participation, treatment may continue for approximately 5 years after the last participant is enrolled in the study or until afimkibart is commercially available in that region, whichever is earlier.

The end of the GA45330 study is expected to occur approximately 30 weeks after the last participant is enrolled.

In the GA45330 study, the total maximum duration of study participation for an individual is expected to be approximately 30 weeks without OLE participation. With OLE participation, treatment may continue for approximately 5 years after the last participant is enrolled in the study or until afimkibart is commercially available in that region, whichever is earlier.

### Example 3. UC Study Populations

Approximately 400 participants with UC are enrolled during the global enrollment phase of the GA45329 study. Approximately 350 participants with UC are enrolled during the global enrollment phase of the GA45330 study. Inclusion and exclusion criteria relevant to both studies are provided below.

### A. Inclusion Criteria

Participants are eligible to be included in the GA45329 study and/or the GA45330 study only if all of the following criteria apply:

i. General Inclusion Criteria.
Age ≥18 to ≤80 years. Patients aged ≥16 to <18 years who are at Tanner Stage 5 (the final adult form) may be eligible to participate in the study where locally permissible (e.g., if permitted by local guidelines and regulations). See Marshall and Tanner, Arch Dis Childh., 44 (291), 1969 and Marshall and Tanner, Arch Dis Childh., 45 (13), 1970.
Body weight ≥40 kg.
   ii. Ulcerative Colitis-Specific Inclusion Criteria
Confirmed diagnosis of ulcerative colitis (UC) with supportive clinical, endoscopic, and histopathological evidence.
Active UC confirmed by endoscopy (flexible sigmoidoscopy or colonoscopy) extending ≥15 cm from the anal verge. Participants with proctitis only (with a minimum extent of inflammation 5 cm proximal to the anal verge) at baseline are capped at 10% of the total enrollment.
Moderately to severely active UC, defined as an mMS of 5 to 9 points, including a Mayo endoscopic score(ES) of 2 or 3, confirmed through centrally read endoscopy performed either (i) during the screening period or (ii) before the screening period (independently of the study; “standard of care (SOC) endoscopy”), within 4 weeks of randomization, and in patients who already have an established UC diagnosis. If performed before screening, the SOC endoscopy must have a video recording available and in a format that is suitable for central reading. Participants must not have had any new medications started in between an SOC endoscopy and randomization. All UC concomitant medications must have been stabilized prior to SOC endoscopy.
Screening for colorectal cancer (CRC) (performed according to local standards) for all participants. For patients with extensive colitis or pancolitis of greater than 8 years' duration or patients with left-sided colitis of greater than 12 years' duration, a surveillance colonoscopy must be performed within 12 months prior to screening. For all other patients, must be up-to-date with CRC surveillance (according to CRC risks and local standards). Any adenomatous polyps must be completely removed according to routine practice prior to their first dose of study drug.
   iii. Reproductive Inclusion Criteria.
For female participants of childbearing potential: agreement to remain abstinent (refrain from heterosexual intercourse) or use adequate contraception and agreement to refrain from egg donation or undergo fertility treatment during the treatment period and for 95 days after the final dose of afimkibart.
For male participants: agreement to remain abstinent (refrain from heterosexual intercourse) or use a condom, and agree to refrain from donating sperm.
   iv. Prior Medications Inclusion Criteria

Participants enrolled in the GA45329 or GA45330 study must have had at least one of the following treatments in the past with inadequate response, loss of response, and/or intolerance.

Inadequate response is defined as having signs and symptoms of persistently active disease despite completing at least the approved dosing regimen in the product label.

Intolerance may include, but is not limited to, infusion-related reactions, injection site reactions, rash, serum sickness, hepatic abnormalities, demyelination, congestive heart failure, and infections. There is no minimum requirement for dose or duration if a potential participant was determined to be intolerant to prior treatment.

Loss of response is defined as the recurrence of signs and symptoms of active disease during approved treatment following prior clinical benefit (discontinuation despite clinical benefit does not qualify as having failed or being intolerant to UC Advanced therapy).

The medication used to qualify the participant for entry into this category must be approved for the treatment of UC, including biosimilars. Participants previously exposed to investigational therapies for the treatment of UC must still meet the inclusion criteria “Conventional Therapy Failure” or “Advanced Therapy Failure.”

### Conventional Therapy Failure

Steroids (e.g., systemic prednisone, oral budesonide).
The following definitions are used as guidelines for the use of corticosteroids in the trials:
   Corticosteroid refractory: Persistent active disease despite treatment with at least one 4-week induction regimen, including a starting dose of ≥20 mg of oral prednisone (or equivalent) for at least 2 weeks or IV prednisone for ≥5 days, or persistently active disease after at least 4 weeks of oral budesonide given 9 mg/day.
   Corticosteroid dependent: (i) Unable to reduce steroids below the equivalent of prednisolone 10 mg/day within 3 months of starting steroids, without recurrent active disease, or (ii) relapse within 3 months of stopping steroids.
   Corticosteroid intolerant: History of intolerance to corticosteroids (including but not limited to Cushing syndrome, osteopenia/osteoporosis, hyperglycemia, insomnia, infection).
At least 12 weeks of an immunomodulator, which can include:
   ≥1.5 mg/kg/day of oral azathioprine (AZA) (or as per local standard of care (SOC)).
   ≥0.75 mg/kg/day of oral 6-mercaptopurine (6-MP) (or as per local SOC).
   ≥15 mg/week of intramuscular or SC methotrexate (MTX).
   Persistent signs and symptoms of active disease despite a 6-TG level of ≥230 pmol/8×10⁸ RBCs during at least one 12-week regimen of oral AZA or 6-MP at a stable or increasing dose.
   History of intolerance to AZA, 6-MP, or MTX (including, but not limited to, nausea/vomiting, abdominal pain, pancreatitis, LFT abnormalities, lymphopenia, TPMT genetic mutation, infection).
At least 4 weeks of an oral aminosalicylate, which can include a minimum dose of the following:
   2.4 g/day of mesalamine (or as per local SOC)·
   4.0 g/day of sulfasalazine (or as per local SOC)
   1.0 g/day of olsalazine (or as per local SOC)
   6.75 g/day of balsalazide (or as per local SOC)

The conventional therapy failure population also includes patients who have received advanced therapy (biologics or small molecules) in the past but stopped therapy based on reasons other than failure (e.g., change in reimbursement coverage, well-controlled disease).

### Advanced Therapy Failure.

Anti-tumor necrosis factor (TNF) agents, including and not limited to the following:
   At least one 6-week induction regimen of infliximab (≥5 mg/kg IV at 0, 2, and 6 weeks or per local label) or equivalent biosimilar.
   At least one 8-week induction regimen of adalimumab (one 160 mg SC dose followed by one 80 mg SC dose (or one 80 mg SC dose in countries where this dosing regimen is allowed) followed by one 40 mg SC dose at least 2 weeks apart or per local label) or equivalent biosimilar.
   At least one 2-week induction regimen of golimumab (one 200 mg SC dose followed by one 100 mg SC dose at least 2 weeks apart or per local label).
Anti-integrins, including and not limited to the following:
   At least one 6-week induction regimen of vedolizumab (300 mg IV at 0, 2, and 6 weeks or per local label).
Anti-IL12/IL23, including and not limited to the following:
   At least one 8-week induction regimen of ustekinumab (a single IV dose using weight-based dosing (260 mg for participants with body weight≤55 kg; 390 mg for participants with body weight >55 kg to ≤85 kg; 520 mg for participants with body weight >85 kg or per local label) (single weight-based dose)) or equivalent biosimilar.
   At least one 8-week regimen of mirikizumab (300 mg IV at Weeks 0, 4, and 8 or per local label).
   At least one 8-week induction regimen of risankizumab (1200 mg IV at Weeks 0, 4, and 8, or per local label).
   At least one 8-week induction regimen of guselkumab (200 mg IV at Weeks 0, 4, and 8, or per local label).
Janus kinase (JAK) inhibitors, including and not limited to the following:
   At least one 8-week induction course of upadacitinib (45 mg orally daily or per local label).
   At least one 8-week induction course of tofacitinib (10 mg orally twice daily of the immediate-release tablet or 22 mg orally daily of the extended-release tablet or per local label).
Sphingosine-1-phosphate (S1P) receptor modulators, including and not limited to the following:
   At least one 10-week induction course of ozanimod (0.92 mg orally daily).
   At least one 12-week induction course of etrasimod (2 mg orally daily).

### B. Exclusion Criteria

Potential participants are excluded from the studies if any of the following criteria apply:

i. Inflammatory Bowel Disease Exclusion Criteria.
Severe UC as evidenced by any of the following:
   Hospitalization for the treatment of UC≤2 weeks prior to screening or, in the physician's judgement, is likely to require hospitalization for medical care or surgical intervention of any kind for UC (e.g., colectomy) during the study.
   Current evidence of fulminant colitis, toxic megacolon, or recent history (within 6 months) of toxic megacolon, or bowel perforation.
   Prior extensive colonic resection, subtotal, or total colectomy, or planned surgery for UC during the study.
Current diagnosis of Crohn's disease (CD), abdominal/intrabdominal/perianal fistula and/or abscess, indeterminant colitis, IBD-unclassified, microscopic colitis, ischemic colitis, infectious colitis, radiation colitis, or active diverticular disease.
Presence of an ostomy or ileoanal pouch.
Current diagnosis or suspicion of primary sclerosing cholangitis.
   ii. Medical History Exclusion Criteria
Lack of peripheral venous access.
Any major surgery within 6 weeks prior to screening or a major surgery planned during the study.
Significant uncontrolled medical comorbidity (such as cardiac, pulmonary, renal, hepatic, or gastrointestinal disorders (excluding UC)), psychiatric, or other condition that, in the opinion of the investigator, would confound the study results, compromise patient safety, or interfere with the potential participant's provision of informed consent or compliance with trial procedures.
Pregnancy or breastfeeding, or intention of becoming pregnant during the study or within 95 days after the final dose of afimkibart.
Any condition that would preclude endoscopic evaluation.
Past or current evidence of definite low-grade or high-grade colonic dysplasia or adenomas or neoplasia not completely removed.
History of malignancy within 5 years prior to screening visit, with the exception of malignancies adequately treated with resection for non-metastatic basal cell or squamous cell cancer or in situ cervical cancer.
History of alcohol, drug, or chemical abuse <1 year prior to screening.
History of blood transfusion within 30 days prior to screening.
   iii. Infection or Infection Risk Exclusion Criteria.
Any clinically significant infection <4 weeks prior to randomization that has not resolved, and/or that required hospitalization and/or IV antibiotics. Any clinically significant infection that was opportunistic in nature is not permitted within 3 months prior to randomization.
Evidence of or treatment for Clostridioides difficile (C. difficile; formerly known as Clostridium difficile) as assessed by detection of C. difficile toxin within 30 days prior to randomization (Week 0, Day 1) or other enteric pathogens (as assessed by stool culture/sensitivity and ova and parasite evaluation) within 30 days prior to randomization (Week 0, Day 1).
Any diagnosis of cytomegalovirus (CMV) colitis in the past 30 days (including diagnosis during screening). Laboratory confirmation of CMV from a colon biopsy sample is required during screening evaluation only if clinical suspicion is high and to determine the need for CMV treatment.
Confirmation of HIV infection (e.g., positive HIV test) at screening.
Positive test results for hepatitis B infection at screening, defined as meeting either of the following criteria: (i) Positive hepatitis B surface antigen (HbsAg) test at screening, and (ii) Quantitative HBV DNA above the lower limit of quantification in patients with a negative hepatitis B surface antibody (HbsAb) test and positive total hepatitis B core antibody (HbcAb) test.
Positive hepatitis C virus (HCV) antibody test at screening.
Positive for tuberculosis (TB) during screening or within 3 months prior to screening, defined as a positive QUANTIFERON® TB-Gold Test (QFT) or, if QFT is not available, a positive purified protein derivative (PPD) skin test according to local guidelines or regulations or other locally approved TB enzyme-linked immunosorbent assay (ELISA) tests (e.g., T-SPOT®), with the following exceptions: (i) Potential participants with a history of Bacillus Calmette-Guerin (BCG) vaccination who have a positive PPD skin test will not be excluded if they have a negative QFT at screening; and (ii) Potential participants who have a positive or indeterminate QFT and those with no history of BCG vaccination who have a positive PPD skin test will not be excluded if they meet all of the following criteria: No symptoms that are consistent with TB, documented history of a completed course of adequate prophylaxis (completed treated for latent TB) per local standard of care prior to randomization (Week 0, Day 1), no known exposure to a case of active TB after most recent prophylaxis, and no evidence of active TB on chest X-ray performed during screening or within 3 months prior to screening.
History of organ transplant.
Acquired or congenital immunodeficiency.
   iv. Laboratory Results Exclusion Criteria.
Clinically significant abnormality on laboratory tests during screening (hematology, serum chemistry, and urinalysis) that, in the opinion of the investigator, may pose an additional risk in administering study treatment to the potential participant.
ALT, AST, or ALP ≥2.5× upper limit of normal (ULN), total bilirubin ≥1.5×ULN, or presence of abnormalities in synthetic liver function tests judged to be clinically significant by the investigator. Patients with an established diagnosis of Gilbert's syndrome (i.e., with required source documentation showing unconjugated hyperbilirubinemia with no evidence of hemolysis) with total bilirubin levels <3× ULN can be included.
ANC <1.5×10⁹/L (1500/μL) with one exception: Participants with documented benign ethnic neutropenia (BEN): ANC <1.3×10⁹/L (1300/μL).
Platelet count <100,000 μL.
Hemoglobin <8 g/dL.
Absolute lymphocyte count <500/μL.
Estimated glomerular filtration rate (eGFR)<30 mL/min/1.73 m².
   v. Prohibited Medications Exclusion Criteria

Prohibited medications are any of the following, related to previous or current treatment:
Use of approved UC treatments including approved oral small molecule (e.g., S1P receptor modulator, JAK inhibitor) treatments within 2 weeks prior to screening endoscopy (or SOC endoscopy), or approved biologic agents within 8 weeks or 5 half-lives prior to randomization (Week 0, Day 1), whichever is longer. If there is proper documentation of undetectable drug level measured by a commercially available assay for any of the approved biologics, there is no minimum washout prior to randomization (Week 0, Day 1).
Use of any investigational or experimental therapy within approximately 30 days for non-biologic therapy or 8 weeks for biologic therapy OR 5 half-lives (whichever is longer) prior to randomization (Week 0, Day 1).
Any immunosuppressive therapy including B-cell depleting agents (e.g., natalizumab, rituximab) or other lymphocyte depleting agents (e.g., alemtuzumab), or any other immunosuppressive agents (e.g., those where live vaccines are contraindicated) within 1 year prior to screening or intent to receive during the study.
Oral prednisone >20 mg/day within 2 weeks prior to screening endoscopy (or SOC endoscopy) or not on stable dose of ≤20 mg prednisone for ≥2 weeks prior to screening endoscopy (or SOC endoscopy) or intent to receive during the study.
Treatment with IV corticosteroids ≤2 weeks prior to screening endoscopy (or SOC endoscopy) or intent to receive during the study, with the exception of a single administration of IV steroid for potential infusion-related reaction (IRR) management.
Presence of conditions other than UC (e.g., uncontrolled asthma) that could require treatment with >20 mg/day of prednisone (or equivalent) during the course of the study.
Treatment with corticosteroid enemas or suppositories and/or topical (rectal) 5-aminosalicyclic acid (5-ASA) preparations ≤2 weeks prior to screening endoscopy (or SOC endoscopy) or intent to receive during the study.
Treatment with topical rectal traditional medicine (e.g., Chinese medicine), herbal enemas, or suppositories ≤2 weeks prior to screening endoscopy (or SOC endoscopy) or intent to receive during the study.
Treatment with approved oral traditional medicine (e.g., Chinese medicine) ≤4 weeks prior to screening endoscopy (or SOC endoscopy) or intent to receive during the study.
Transplant/stem cell therapy at any time prior to randomization (Week 0, Day 1) or intent to receive during the study.
Treatment ≤16 weeks prior to randomization (Week 0, Day 1) with cyclosporine, tacrolimus, sirolimus, or mycophenolate mofetil, or intent to receive during the study.
Apheresis ≤2 weeks prior to screening endoscopy (or SOC endoscopy) or intent to receive during the study.
Receipt of fecal microbial transplantation within 4 weeks prior to randomization (Week 0, Day 1).
Known exposure to anti-TL1A (afimkibart (RVT-3101)/PF-06480605) or any type of anti-TL1A therapy.
Receipt of a live or attenuated vaccine $4 weeks prior to screening endoscopy (or SOC endoscopy) or intent to receive during the study; use of non-live (inactivated) vaccines are allowed.
Chronic (e.g., >7 days) nonsteroidal anti-inflammatory drug (NSAID) use; occasional use of NSAIDs and acetaminophen is permitted (e.g., for headache, arthritis, myalgias, or menstrual cramps). Aspirin ≤325 mg/day is permitted.
Treatment with immunoglobulin or blood products within 4 weeks prior to screening, or any condition that is likely to require such treatment during the course of the study.
IV antibiotics ≤4 weeks prior to randomization (Week 0, Day 1).
Previous severe allergic reaction (NCI CTCAE v5.0 Grade 3 or higher) or anaphylactic reaction to biologic agents or to any excipients of the study drug.

### Example 4. UC Study Treatment, Other Treatments Relevant to Study Design, and Concomitant Therapy

In the GA45329 and GA45330 studies, afimkibart (500 mg) or placebo is administered intravenously (IV) at Weeks 0, 2, 6, and 10 (induction phase). In the GA45329 study, afimkibart (450 mg) or placebo is then administered subcutaneously (SC) every 4 weeks (Q4W) from Week 12 through Week 52 (maintenance phase). The maintenance phase is omitted in the GA45330 study. In the open-label extension phase of both studies, afimkibart is administered either 450 mg SC Q4W or 450 mg SC every 2 weeks (Q2W). Modification of the study drug dose is not permitted during the double-blind phases of the studies. However, dose intensification or de-escalation (either from Q4W to Q2W or from Q2W to Q4W, respectively) may be permitted during the OLE phase. Any other dosing frequencies (e.g., weekly dosing) are not permitted.

Table 13 provides a description of assigned study treatments for the GA45329 and GA45330 studies.

TABLE 13
Study Treatment Description

| | Afimkibart | Placebo |
|---|---|---|
| Use | Experimental | Placebo comparator |
| Drug form | Solution for infusion/injection | Solution for infusion/injection |
| Unit dose | 225 mg/1.5 mL (150 mg/mL) | Not applicable |
| strength | | |
| Dosage levels | Induction: 500 mg IV at Weeks 0, 2, 6, and 10 | Not applicable |
| for GA45329 | Maintenance: 450 mg SC every 4 weeks (Q4W) | |
| study | Open-Label Extension (OLE): 450 mg SC every 2 | |
| | weeks (Q2W) or Q4W | |
| Dosage levels | Induction: 500 mg IV at Weeks 0, 2, 6, and 10 | Not applicable |
| for GA45330 | OLE: 450 mg SC Q2W or Q4W | |
| study | | |
| Route of | Intravenous infusion or subcutaneous injection | Intravenous infusion or |
| administration | | subcutaneous injection |

Intravenous administration of afimkibart is performed in a monitored setting where there is immediate access to trained personnel and adequate equipment and medicine to manage potentially serious reactions.

At applicable sites, SC study treatment in the OLE phase may be administered by a trained nursing professional at the participant's home or another suitable location (mobile nursing (MN) visits). The MN administration is available after the first Q3M visit in Year 1 of the OLE phase.

The IV infusion is delivered over 60 (±10) minutes. The post-dose observation period for IV infusions is 60 minutes.

For participants who experience an infusion-related reaction (IRR), subsequent infusions may be given in accordance with institutional/local clinical guidelines or IRR management guidance including, e.g., slowing the infusion rate, interrupting infusion, and/or administering premedication (which may include acetaminophen (or paracetamol), an antihistamine (e.g., diphenhydramine) and/or a single dose of IV corticosteroids).

The post dose observation period for SC injection is 30 minutes, with the exception of the first 12 weeks of the OLE phase, which have a post-dose observation period of at least 60 minutes. Visits in the first 12 weeks of the OLE are not eligible for MN, i.e., the first 3 doses for participants following the Q4W dose regimen and the first 6 doses for participants following the Q2W dose regimen are not eligible for MN.

### A. Treatment Assignment

GA45329 and GA45330 are randomized, double-blind studies. In each study, participants are randomly assigned to one of two treatment arms: afimkibart or placebo. Randomization occurs in a 3:2 ratio through use of a permuted-block randomization method to ensure a balanced assignment to each treatment arm. Randomization is stratified by advanced UC therapy experience (naïve or prior exposure to biologic, S1P receptor modulator, or JAK inhibitor), baseline corticosteroid use, and baseline disease activity (moderate, defined by mMS 5 or 6, or severe, defined by mMS 7 to 9).

### B. Dose Modification

Modification of the study drug dose is not permitted during the double-blind phase or phases of the studies. However, dose intensification or de-escalation (either from Q4W to Q2W or from Q2W to Q4W, respectively) may be permitted during the OLE phase. Any other dosing frequencies (e.g., weekly dosing) are not permitted.

### C. Concomitant Therapy

Permitted and prohibited concomitant therapies for UC are outlined in Table 14. Permitted and prohibited concomitant therapies not related to UC are outlined in Table 15. Concomitant medication restrictions apply to all phases of the studies, including the OLE phase. In general, investigators may manage a participant's care (including preexisting conditions) through use of supportive therapies, as clinically indicated and per local standard practice, with the exception of prohibited therapies defined in Tables 14 and 15.

TABLE 14
Permitted and Prohibited Concomitant Therapies for Ulcerative Colitis

| Therapy | Permitted | Prohibited |
|---|---|---|
| Anti- | Oral 5-ASA if on stable dose | Oral 5-ASA initiation or dose change |
| inflammatories | for ≥2 weeks prior to screening | throughout the duration of the study. |
| | endoscopy (or SOC | Topical (rectal) 5-ASA ≤2 weeks prior to |
| | endoscopy); stable dose | screening endoscopy (or SOC |
| | throughout the study is | endoscopy) and throughout the |
| | permitted unless toxicity | duration of the study. |
| | where dose should be | IV corticosteroids and rectal |
| | discontinued. | corticosteroids (i.e., enemas or |
| | Oral prednisone ≤20 mg/day (or | suppositories) ≤2 weeks prior to |
| | dose equivalent of other oral | screening endoscopy (or SOC |
| | corticosteroids, e.g., oral | endoscopy) and for the duration of the |
| | budesonide ≤6 mg/day or | study, with the exception of a single |
| | oral budesonide MMX ≤9 | administration of IV steroid for |
| | mg/day) if on stable dose 2 | potential IRR management. |
| | weeks prior to screening | Initiation of oral corticosteroids from |
| | endoscopy (or SOC | randomization (Week 0, Day 1) and for |
| | endoscopy). Stable dose | the duration of the study. |
| | must continue through the | |
| | induction phase. Oral | |
| | corticosteroid tapering must | |
| | be initiated at Week 12. | |
| | Oral prednisone ≥20 mg/day is | |
| | allowed at screening if | |
| | tapered down to a stable | |
| | dose of ≤20 mg/day by 2 | |
| | weeks prior to screening | |
| | endoscopy (or SOC | |
| | endoscopy). | |
| Immuno- | AZA, 6-MP, or MTX if on stable | Cyclosporine, tacrolimus, sirolimus or |
| suppressants | dose 8 weeks prior to | mycophenolate mofetil ≤16 weeks |
| | randomization (Week 0, Day | prior to randomization (Week 0, Day 1) |
| | 1) and continue stable | and throughout the duration of the |
| | dosing throughout the | study. |
| | duration of the study; | |
| | discontinuation of | |
| | concomitant medication | |
| | permitted if the dose is | |
| | reduced or discontinued due | |
| | to toxicity. | |
| Advanced | N/A. | Small molecules (S1P receptor |
| Therapies | | modulators and JAK inhibitors) ≤2 |
| | | weeks prior to screening |
| | | endoscopy (or SOC endoscopy) and |
| | | throughout duration of the study. |
| | | Biologics (anti-TNF, anti-integrin, |
| | | anti-interleukin including biosimilars) |
| | | within 8 weeks or 5 half-lives prior to |
| | | randomization (Week 0, Day 1), |
| | | whichever is longer, and throughout |
| | | the duration of the study. Note: If |
| | | there is proper documentation of |
| | | undetectable drug level measured by a |
| | | commercially available assay for any |
| | | of the approved biologics there is no |
| | | minimum washout prior to |
| | | randomization (Week 0, Day 1). |
| | | Use of any investigational or experimental |
| | | therapy within approximately 30 days |
| | | for non-biologic therapy or 8 weeks for |
| | | biologic therapy OR 5 half-lives |
| | | (whichever is longer) prior to |
| | | randomization (Week 0, Day 1). |
| Other | Oral probiotics if dose stable ≥2 | Initiation of UC-related antibiotics (oral or |
| Therapies | weeks prior to baseline | IV) throughout the duration of the |
| | and continue stable dosing | study. |
| | throughout the duration of | Anti-TL1A agents at any time prior to or |
| | the study. | during the study (except afimkibart |
| | UC-related antibiotics if stable ≥2 | administered as study treatment in this |
| | weeks prior to screening | study). |
| | endoscopy (or SOC | Continued tube feeding, exclusive enteral |
| | endoscopy) and through | nutrition or defined formula diets, |
| | randomization (Week 0, Day | and/or parenteral alimentation/nutrition |
| | 1). | as treatment for UC ≤2 weeks prior to |
| | | screening endoscopy (SOC |
| | | endoscopy) or during the study. |

5-ASA = 5-aminosalicylic acid; 6-MP = 6-mercaptopurine; AZA = azathioprine; IRR = infusion-related reaction; JAK = Janus kinase; MMX = multi matrix; MTX = methotrexate; NA = not applicable; S1P = sphingosine-1-phosphate; SOC = standard of care; TL1A = tumor necrosis factor-like ligand 1A; TNF = tumor necrosis factor; UC = ulcerative colitis.

TABLE 15
Permitted and Prohibited Concomitant Therapies Not Related to Ulcerative Colitis

| Permitted | Prohibited |
|---|---|
| Occasional use of NSAIDs, acetaminophen, | Any investigational or experimental therapy |
| and aspirin ≤325 mg/day. | within 30 days for non-biologic therapy or |
| Non-live (inactivated) vaccines. | 8 weeks for biologic therapy or 5 half-lives |
| CYP substrates with a narrow therapeutic | prior to randomization (Week 0, Day 1), |
| window including but not | whichever is longer. |
| limited to aminoglycosides, ciclosporin, | Note: If there is proper documentation of |
| carbamazepine, digoxin, digitoxin, | undetectable drug level measured by a |
| flecainide, lithium, phenytoin, | commercially available assay for any of the |
| phenobarbital, rifampicin, theophylline, | approved biologics, there is no minimum |
| and warfarin. | washout prior to randomization (Week 0, |
| Monitoring the effect of drug | Day 1) and throughout the study. |
| concentration of the | Any immunosuppressive therapy including B- |
| concomitant treatment (on initiation | cell depleting agents (e.g., natalizumab, |
| or discontinuation of afimkibart) is | rituximab) or other lymphocyte depleting |
| strongly recommended. | agents (e.g., alemtuzumab), or any other |
| | immunosuppressive agents (e.g., those |
| | where live vaccines are contraindicated) |
| | within 1 year prior to screening and |
| | throughout the study. |
| | Topical rectal traditional medicine (e.g., |
| | Chinese medicine), herbal enemas, or |
| | suppositories ≤2 weeks prior to screening |
| | endoscopy (or SOC endoscopy) and |
| | throughout the study. |
| | Treatment with approved oral traditional |
| | medicine (e.g., Chinese medicine) ≤4 |
| | weeks prior to screening endoscopy (or |
| | SOC endoscopy) and throughout the study. |
| | Fecal microbial transplantation within 4 weeks |
| | prior to randomization (Week 0, Day 1). |
| | Live or attenuated vaccines within 4 weeks |
| | prior to screening endoscopy (or SOC |
| | endoscopy) and throughout the study. |
| | IV antibiotics ≤4 weeks prior to randomization |
| | (Week 0, Day 1). |
| | Apheresis ≤2 weeks prior to screening |
| | endoscopy (or SOC endoscopy) and |
| | throughout the study. |
| | Immunoglobulin or blood products within |
| | 4 weeks prior to screening endoscopy (or |
| | SOC endoscopy), or any condition that is |
| | likely to require such treatment during the |
| | course of the study. |
| | Transplant/stem cell therapy at any time prior |
| | to randomization (Week 0, Day 1) and |
| | throughout the study. |
| | Chronic (e.g., >7 days) use of NSAIDs, |
| | acetaminophen and/or aspirin >325 |
| | mg/day during the study. |

CYP = cytochrome P450; NSAID = non-steroid anti-inflammatory drug; SOC = standard of care.

### D. Corticosteroid Tapering

During the 12-week induction phase of both studies, participants are to maintain their stable baseline corticosteroid dose.

In the GA45329 study, following their Week 12 assessment, participants begin corticosteroid tapering during the maintenance phase.

In the GA45330 study, following their Week 12 assessment, and at the investigator's discretion, participants begin corticosteroid tapering within the first 12 weeks of entering the open-label extension phase.

The recommended tapering schedule for oral corticosteroids is shown in Table 16.

TABLE 16
Tapering Schedule for Oral Corticosteroids

| Corticosteroid | Dose | | Tapering Rate |
|---|---|---|---|
| Oral prednisone or | >10 | mg/day | Taper daily dose by 5 mg/week until receiving 10 |
| equivalent | | | mg/day, and then continue tapering at 2.5 |
| | | | mg/week until 0 mg/day. |
| | ≤10 | mg/day | Taper daily dose by 2.5 mg/week until 0 mg/day. |
| Oral budesonide | ≤6 | mg/day | Taper tablets to 3 mg/day for 2 weeks and then |
| | | | discontinue. |
| Oral budesonide | ≤9 | mg/day | Taper tablets to 9 mg every other day for |
| MMX | | | 2 weeks, followed by 9 mg every third day for 2 |
| | | | weeks, and then discontinue. |

For participants who cannot tolerate the corticosteroid taper without recurrence of UC clinical symptoms or steroid withdrawal, the corticosteroid dose may be increased (up to the dose at trial entry if required) during the study, but tapering should begin again within 2 weeks.

### Example 5. UC Study Assessments and Procedures

### A. Efficacy Assessments

In the GA45329 and GA45330 studies, patient-reported outcome (PRO) and clinician-reported outcome (ClinRO) instruments are completed to assess the treatment benefit of afimkibart. In addition, PRO instruments will enable the capture of each participant's direct experience with afimkibart. PRO data are collected through the instruments summarized in Table 17 and described below.

In the GA45329 study, Week 12 efficacy assessments are to be completed prior to the first Week 12 dose of SC study treatment in the maintenance phase.

In the GA45330 study, Week 12 efficacy assessments are to be completed prior to the first Week 12 dose of SC study treatment in the OLE phase.

TABLE 17
Patient-Reported Outcome Instruments

| PRO Instrument | Recall Period | |
|---|---|---|
| Stool frequency | 24 | hours |
| Rectal bleeding | | |
| Bowel urgency | | |
| Abdominal pain | | |
| Functional Assessment of Chronic Illness | 7 | days |
| Therapy-Fatigue (FACIT-F) | | |
| Inflammatory Bowel Disease Questionnaire (IBDQ) | 2 | weeks |
| Patient Global Impression of Change (PGIC) | Current versus | |
| | baseline | |
| Patient Global Impression of Severity (PGIS) | 7 | days |
| EuroQol 5-Dimension 5-Level (EQ-5D-5L)^{a} | Current | |
| Work Productivity and Activity Impairment | 7 | days |
| Questionnaire:Ulcerative Colitis (WPAI:UC) | | |

^{a}This patient-reported instrument is used to evaluate health economics rather than efficacy.

i. Mayo Score

Variants of the Mayo Score are evaluated at specified timepoints as a composite based on up to four assessments, described in further detail below: stool frequency subscore (SFS), rectal bleeding subscore (RFS), physician's global assessment (PGA), and endoscopic subscore(ES). Each of these assessments has scoring that ranges from 0 to 3, with higher values indicating greater severity.

The composite score variants are defined as follows.
Mayo Score (MS): Sum of all four subscores, with range 0-12.
Modified Mayo Score (mMS): Sum of SFS, RBS, and ES, with range 0-9.
Partial Mayo Score (pMS): Sum of SFS, RBS and PGA, with range 0-9.
Partial modified Mayo Score (pmMS): Sum of SFS and RBS, with range 0-6.

Throughout, ES is defined to align with the modified version of the scoring system, where the presence of any friability should correspond to a subscore of at least 2.

ia. Stool Frequency and Rectal Bleeding

Stool frequency and rectal bleeding are single-item self-reported assessments, both with a 24-hour recall period and scored grading on a 4-point Likert scale. Stool frequency assesses the number of trips to the toilet with a bowel movement or passage of blood and/or mucus, relative to the patient's normal frequency. Rectal bleeding assesses the most severe amount of blood passed per rectum among these trips to the toilet.

The “normal” reference for stool frequency corresponds to the number of stools in a 24-hour period when in remission or (if the disease has never entered remission) prior to initial onset of UC signs and symptoms leading to UC diagnosis. This number is recorded for each participant in screening.

The corresponding subscores (SFS and RBS, respectively) are calculated as an average over 7 days prior to the relevant timepoint.

SFS is quantified as follows:

With normal defined at baseline, with respect to each individual participant:
0=Normal number of stools for this patient.
1=1-2 more stools than normal.
2=3-4 more stools than normal.
3=5 or more stools than normal.

RBS is quantified as follows:

Representing the worst rectal bleeding observed by the participant that day:
0=No blood seen or no bowel movement.
1=Stool with streaks of blood.
2=Stool with more than streaks of blood.
3=Blood alone passed.
   Ib. Physician's Global Assessment

The subscore reported for PGA should reflect the clinician's assessment of the participant's current overall status, acknowledging the other criteria including the patient's daily abdominal discomfort, general sense of well-being, physical examination findings, and performance status (Schroeder et al., NEJM, 317 (26): 1625-1629, 1987).

PGA is quantified as follows:
0=Normal.
1=Mild.
2=Moderate.
3=Severe.
   ic. Endoscopy

All participants undergo either a colonoscopy or flexible sigmoidoscopy at specified visits or as part of SOC (if done before the screening period, independently of the study). At screening, requirements for the extent and timing of the endoscopy assessment are determined by UC-specific study inclusion criteria.

ES should reflect the worst appearance of the mucosa on flexible sigmoidoscopy or colonoscopy, on a 4-point scale. Consistent with regulatory guidance, this scale excludes mild friability from the features characteristic of ES=1; any friability apparent on the video recording should be reported as ES ≥2. ES evaluated by blinded central reading is used to determine inclusion criteria, efficacy endpoints, and disease worsening criteria.

Biopsies are collected during withdrawal of the endoscope from the bowel.

ES is quantified as follows:
0=Normal appearance of mucosa.
1=Mild disease (erythema, decreased vascular pattern, no friability).
2=Moderate disease (marked erythema, absent vascular pattern, friability, erosions).
3=Severe disease (spontaneous bleeding, ulceration).
   ii. Geboes Grading Scale

The Geboes Grading Scale is a seven-item classification system to evaluate histological activity in UC, with items graded from least to most severe features of inflammation. (Geboes et al., Gut, 47:404-409, 2000). Each grade is assigned a subgrade ranging from 0 to 3 or 4, with higher values associated with greater severity of the corresponding features.

Grading is carried out by central reading, using slide images processed from colonic biopsies taken during endoscopy at specified visits.

iii. Bowel Urgency

Bowel urgency is a single-item self-reported assessment of sudden or immediate need to have a bowel movement in the past 24 hours. The item response is reported on a 4-point Likert scale, from “None” to “Severe.”

iv. Abdominal Pain

Abdominal pain is a single-item self-reported assessment of severity in abdominal pain in the past 24 hours. The item response is reported on a 4-point Likert scale, from “None” to “Severe.”

v. Functional Assessment of Chronic Illness Therapy-Fatigue

The Functional Assessment of Chronic Illness-Fatigue (FACIT-F; Version 4) is a 13-item self-reported assessment of fatigue. FACIT-F has been validated for use in a variety of conditions, including anemia and IBD (Yellen et al., J Pain Symptom Manage, 13:63-74, 1997; Cella et al., J Rheum, 32:811-819, 2005; Lai et al., J Rheumatol, 38:672-679, 2011; Tinsley et al., Aliment Pharmacol Ther, 34:1328-1336, 2011; Acaster et al., Health Qual Life Outcomes, 13:60, 2015). Each item response option indicates the degree to which a given statement describing the level or impact of fatigue applies in the past 7 days. Response options are graded on a 5-point Likert-type scale, from “Not at all” to “Very much.”

vi. Inflammatory Bowel Disease Questionnaire

The Inflammatory Bowel Disease Questionnaire (IBDQ) is a validated self-reported 32-item assessment of health-related quality of life in patients with IBD (Guyatt et al., Gastroenterology, 96:80410, 1989; Irvine, J Pediatr Gastroenterol Nutr, 28: S23-S27, 1999). The IBDQ covers four domains: bowel symptoms (10 questions); systemic symptoms including sleep disorders and fatigue (5 questions); emotional function such as depression, aggression, and irritation (12 questions); and social function, meaning the ability to participate in social activities and work (5 questions). Each question has a recall period of the past 2 weeks. Response options are graded on a 7-point Likert-type scale.

vii. Patient Global Impression of Change

The Patient Global Impression of Change (PGIC) is a single-item self-reported assessment of the change in overall UC symptoms, from the start of the study to current status. The item response is reported on a 5-point Likert-type scale, from “Much worse” to “Much better.”

viii. Patient Global Impression of Severity

The Patient Global Impression of Severity (PGIS) is a single-item self-reported assessment of the severity of overall UC symptoms. The item response is reported on a 6-point Likert scale, from “None” to “Very severe.”

### B. Biomarker Assessments

The following biomarker samples are collected, as applicable, from screened patients and participants at all pre- and post-treatment timepoints at all sites:
Blood samples for determination of the TL1A biomarker status are collected from all screened patients, and nucleic acids are used to assess predictive biomarkers and may be used to support potential development of diagnostic and other biomarker assays.
For PD biomarker assessments, peripheral blood is collected and processed to assess total STL1A levels.
Non-invasive biomarker measurements of hsCRP and fecal calprotectin are conducted in peripheral blood and stool samples, respectively.
Exploratory peripheral blood, serum, and stool are collected to measure other biomarkers of the TL1A pathway and pathophysiology biomarkers. Methods may include but will not be limited to RNA-sequencing, immunoassays, mass spectrometry, and PCR.
Colonic biopsy tissues are collected at designated endoscopy visits, including if the endoscopy was performed before screening as part of SOC. These colonic biopsies are used for exploratory biomarker determination, which may include but will not be limited to the assessment of local PD biomarkers, TL1A pathway biomarkers, drug exposure, and pathophysiology biomarkers related to inflammation and histology. These assessments may include RNA sequencing, immunoassays, IHC, mass spectrometry, PCR, and spatial imaging methods.

Exploratory biomarker research may include, but will not be limited to, cytokines/chemokines, target and pathway proteins or genes, inflammatory genes or proteins, microbiome and products thereof in blood, serum or plasma, stool, and mucosal tissues. Genomic research may include exploration of germline variants. Genomic profiling may include whole genome sequencing (WGS) or whole exome sequencing (WES) of blood samples.

Biomarkers are assessed at baseline and at subsequent timepoints following administration of afimkibart or matching placebo. Biomarker levels at baseline or over time may be compared with efficacy, other biomarkers, imaging, or safety measurements to assess prognostic or predictive properties. Biomarkers may also be analyzed over time as absolute values and/or percent change relative to baseline over time, and may be compared with efficacy, PK, other biomarkers, or safety measurements to determine PD properties. Exploratory biomarker analyses may include prognostic, predictive, and PD biomarker analyses from DNA/RNA-based assays. After PK or immunogenicity analyses have been completed, any remaining plasma and/or serum may be used for exploratory biomarker research described above.

For determining variants in the TL1A biomarker, a mandatory blood sample for DNA isolation is collected from consenting participants at screening.

### C. Work Productivity and Activity Impairment Questionnaire: Ulcerative Colitis

The Work Productivity and Activity Impairment Questionnaire: Ulcerative Colitis Questionnaire (WPAI: UC) v2.0 is an adaptation of the WPAI: Specific Health Profile, a generic patient-reported measure of absenteeism, presenteeism, work productivity and activity impairment over the past seven days (Reilly et al., Pharmacoeconomics, 4:353-365, 1993). The first question (Q1) asks if the individual is currently employed (yes/no). If the individual indicates “yes”, then he/she is asked to indicate the number of hours missed from work due to his/her UC (Q2), the number of hours missed for other reasons (Q3), and the number of hours worked (Q4). If the answer to Q1 is “yes” and the answer to Q4 is >0, then the individual is asked to indicate the impact of UC on work productivity on a 0-10 numeric rating scale (NRS). All individuals will then be asked to indicate the impact of UC on regular daily activities on a 0-10 NRS (Q6). Four scores (absenteeism, presenteeism, work productivity, and activity impairment) are obtained from the scale and are expressed as impairment percentages, with higher numbers indicating greater impairment and less productivity (i.e., worse outcomes).

### D. EuroQol 5-Dimension 5-Level

The EuroQol 5-Dimension 5-Level (EQ-5D-5L) is a validated self-reported health status questionnaire that is used to calculate a health status utility score for use in health economic analyses (EuroQol Group, Health Policy, 16:199-208, 1990; Brooks, Health Policy, 37:53-72, 1996; Herdman et al., Qual Life Res, 20:1727-1736, 2011; Janssen et al., Qual Life Res, 22:1717-1727, 2013). There are two components to the EQ-5D-5L: a five-item health state profile that assesses mobility, self-care, usual activities, pain/discomfort, and anxiety/depression, as well as a Visual Analog Scale (VAS) that measures health state. The EQ-5D-5L is designed to capture a participant's current health status. Published weighting systems allow for creation of a single composite score of the participant's health status. The EQ 5D-5L is used in the study for informing pharmacoeconomic evaluations.

### Example 6. UC Study Statistical Considerations

This Example provides a summary of the statistical aspects of the GA45329 and GA45330 study designs and the general approach to analysis of endpoints supporting the study objectives.

Analysis of GA45329 study data is anchored upon the following milestones:
Primary completion, given by the date at which the last participant in the maintenance treatment phase completes all Week 52 primary endpoint assessments.
Study completion, given by the date of the last-participant, last-visit across all phases of the study, including open-label extension (OLE).

Analysis of GA45330 study data is anchored upon the following milestones:
Primary completion, given by the date at which the last participant in the induction treatment phase completes all Week 12 primary endpoint assessments.
Study completion, given by the date of the last-participant, last-visit across all phases of the study, including OLE.

### A. Statistical Hypotheses

### GA45329 Study

Under the co-primary objectives for the GA45329 study, it is hypothesized that afimkibart is superior to placebo in inducing and maintaining clinical remission. In statistical testing the relevant null and alternative hypotheses are formulated as follows:

H₀: p_{afimkibart}−p_{placebo}=0 versus H_{A}: p_{afimkibart}−p_{placebo}≠0,

where p_{afimkibart}−p_{placebo} represents the treatment effect (estimand) of interest, the difference in the proportion of patients achieving clinical remission at a particular timepoint (Week 12 or 52) after assignment to afimkibart versus placebo, as described in Example 1.

The GA45329 study is deemed positive if both null hypotheses based on clinical remission at Week 12 and at Week 52 are rejected in favor of afimkibart. Each of these two-sided tests is carried out at the 5% significance level. Should the primary results be positive, testing will proceed to a specified subset of key secondary endpoints (Example 1). All tests are prioritized into a hierarchy of families and evaluated successively, by parallel gatekeeping via the truncated Hochberg multiple testing procedure with truncation fraction 0.5. Such a multiple testing strategy ensures overall type 1 error control at 5% (Dmitrienko et al., Biom J, 50:667-677, 2008).

Tests on secondary endpoints consider null and alternative hypotheses like those defined for the co-primary endpoints above. Inference on primary and secondary endpoints is generally based on a covariate-adjusted standardized estimator of the marginal treatment effect, which is asymptotically normal and yields Wald-type tests using the point and variance estimates (Rosenblum and van der Laan, Int J Biostat, 1 (6), Article 13, 2010).

### GA45330 Study

Under the primary objective for the GA45330 study, it is hypothesized that afimkibart is superior to placebo in inducing clinical remission. In statistical testing the relevant null and alternative hypotheses are formulated as follows:

H₀: p_{afimkibart}−p_{placebo}=0 versus H_{A}: p_{afimkibart}−p_{placebo}≠0,

where p_{afimkibart}−p_{placebo} represents the treatment effect (estimand) of interest, the difference in the proportion of patients achieving clinical remission at Week 12 after assignment to afimkibart versus placebo, as described in Example 1.

The GA45330 study is deemed positive if the above null hypothesis is rejected in favor of afimkibart.

This two-sided test is carried out at the 5% significance level. Should the primary result be positive, testing will proceed to a specified subset of key secondary endpoints (Example 1). All tests are prioritized into a hierarchy of families and evaluated successively, by parallel gatekeeping via the truncated Hochberg multiple testing procedure with truncation fraction 0.5. Such a multiple testing strategy ensures overall type 1 error control at 5% (Dmitrienko et al., Biom J, 50:667-677, 2008).

Tests on secondary endpoints consider null and alternative hypotheses like those defined for the primary endpoint above. Inference on primary and secondary endpoints is generally based on a covariate-adjusted standardized estimator of the marginal treatment effect, which is asymptotically normal and yields Wald-type tests using the point and variance estimates (Rosenblum and van der Laan, Int J Biostat, 1 (6), Article 13, 2010).

i. Sample Size Determination

### GA45329 Study

In the GA45329 study, a total of approximately 400 participants are enrolled and randomly assigned to either afimkibart or placebo under a 3:2 randomization ratio. This will allocate approximately 240 participants to the afimkibart arm and 160 to the placebo arm. Marginal power to reject one of the null hypotheses in primary analysis at the significance level of 5% depends (in part) on the clinical remission rate under placebo treatment. In more recent Phase III UC trials, placebo induction of clinical remission is estimated to be 10% (95% CI: 9% to 13%) but this rate depends on a variety of factors, such as the proportion of participants with prior advanced therapy (Sedano et al., J Crohns Colitis, 16:224-243, 2022).

The placebo rate of clinical remission in maintenance further depends on the maintenance treatment period design: randomized withdrawal or treat-through. Imputation of treat-through placebo rates from randomized withdrawal trials yields estimates ranging from 5%-6% and 12%-17% in patients with and without prior biologic failure, respectively (Welty et al. 2020). Direct estimates under a placebo-controlled treat-through trial enrolling both advanced therapy naïve and experienced participants are available from ULTRA 2 (Sandborn et al. Gastroenterology, 142:257-265.e3, 2012, primary analysis set excluding participants from noncompliant sites) and more recently ELEVATE UC 52 (Sandborn et al., The Lancet, 401:1159-1171, 2023, primary analysis set excluding participants with baseline mMS <5). The observed Week 52 clinical remission rates in the placebo arms of these studies were 9% (95% CI: 6% to 13%) and 7% (95% CI: 4% to 12%), respectively.

Safety objectives further require a placebo arm large enough to ensure that there are placebo-treated participants through the 52-week treatment period. In ULTRA 2 and ELEVATE UC 52, 56/246 (23%) and at least 39/135 (29%) of placebo-treated patients completed 52 weeks of treatment, respectively. Rather than assuming the placebo treatment completion rate directly, the anticipated number of placebo-treated completers can be estimated from assumed maintenance clinical remission rates in the placebo arm, both overall and among treatment completers. In ULTRA 2 and ELEVATE UC 52, the clinical remission rates at Week 52 among treatment completers in the placebo arm were 38% and 23%, respectively.

Under a range of values for these rates, Table 18 gives key operating characteristics for the proposed sample size. Throughout, the treatment effect is fixed at 15%, a value considered clinically meaningful and achievable given the Phase 2 efficacy findings on afimkibart in UC.

TABLE 18
Operating Characteristics for the Proposed Study Design

| | | Anticipated number in |
|---|---|---|
| Afimkibart vs. placebo | Marginal | placebo arm completing |
| treatment effect | power | treatment at Week 52 |
| 30 vs. 15% | 94% | 60-96 |
| 25 vs. 10% | 97% | 40-64 |
| 21 vs. 6% | 99% | 24-38 |

Note:
Marginal power is evaluated using Fisher's exact test, assuming the given effect on either co-primary endpoint, at Week 12 or Week 52. Lower (upper) range value for the anticipated number of participants in the placebo arm who complete treatment at Week 52 is given by the planned placebo arm size of 160, multiplied by the placebo remission rate assuming the given effect at Week 52, and divided by 40% (25%).

Table 18 considers marginal power to detect the given treatment effect on any one of the co-primary endpoints. The corresponding joint power (on both co-primary endpoints) is at least 0.942=88%.

### GA45330 Study

In the GA45330 study, a total of approximately 350 participants are enrolled and randomly assigned to either afimkibart or placebo under a 3:2 randomization ratio. This allocates approximately 210 participants to the afimkibart arm and 140 to the placebo arm. Power to reject the null hypothesis in primary analysis at the significance level of 5% depends (in part) on the clinical remission rate under placebo treatment. In more recent Phase III UC trials, placebo induction of clinical remission is estimated to be 10% (95% CI: 9% to 13%) but this rate depends on a variety of factors, such as the proportion of participants with prior advanced therapy (Sedano et al., J Crohns Colitis, 16:224-243, 2022).

Under a range of values for the placebo rate, Table 19 gives power for the proposed sample size. Throughout, the treatment effect is fixed at 15%, a value considered clinically meaningful and achievable given the Phase II efficacy findings on afimkibart in UC.

TABLE 19
Operating Characteristics for the Proposed Study Design

| Afimkibart vs. placebo | |
|---|---|
| treatment effect | Power |
| 30 vs. 15% | 90% |
| 25 vs. 10% | 95% |
| 21 vs. 6% | 98% |

Note:
Power is evaluated using Fisher's exact test.

### B. Analysis Sets

In both studies, each planned analysis incorporates data from a particular set of participants. These participant analysis sets are broadly defined in Table 20. In general, the relevant data for a given participant in an analysis set may consider all measurements for parameters under analysis, from baseline up to the timing of the endpoint in question, such as Week 12 for induction endpoints and, in the GA45329 study, Week 52 for maintenance endpoints.

TABLE 20
Participant Analysis Sets

| Analysis Set | Description |
|---|---|
| Full | All enrolled participants |
| Efficacy | All randomized participants who were exposed to study treatment, grouped |
| | by assigned treatment arm. |
| Biomarker-evaluable | All randomized participants who were exposed to study treatment and have |
| | TL1A biomarker status measured at screening, grouped by assigned |
| | treatment arm. |
| Safety | All enrolled participants who were exposed to study treatment, grouped by |
| | actual treatment arm (i.e., treatment arm most representative of treatment |
| | received). |
| PK-evaluable | All enrolled participants who received at least one dose of afimkibart and |
| | have at least one concentration value measured, grouped by actual |
| | treatment arm. |
| ADA-evaluable | All enrolled participants who received at least one dose of afimkibart and |
| | have at least one post-baseline anti- afimkibart antibody determination, |
| | grouped by actual treatment arm. |
| PD-evaluable | All enrolled participants who received at least one dose of afimkibart and |
| | have at least one total soluble TL1A concentration value measured, |
| | grouped by actual treatment arm. |

ADA = anti-drug antibodies; PD = pharmacodynamic; PK = pharmacokinetic.

Baseline for a given parameter is generally defined as the last value measured prior to first dose of study drug. Post-baseline measurements assessed outside the planned schedule, at unscheduled or early termination visits, are allocated to a scheduled visit according to prespecified visit windows.

### C. Statistical Analyses

i. General Considerations

All endpoints supporting efficacy, safety, PK, PD, and immunogenicity objectives (Example 1) will be evaluated in the corresponding analysis set (Table 20). To aid in endpoint comparison between afimkibart versus placebo, treatment group summaries or differences thereof with 95% confidence intervals may be generated. In the analysis of efficacy endpoints, these differences will generally correspond to estimates for treatment effects. For other endpoints, analysis may be limited to descriptive treatment group summaries.

### Example 7. Phase III, Multicenter, Double-Blind, Placebo Controlled Studies to Assess the Efficacy and Safety of Therapy with Afimkibart in Patients with Moderately to Severely Active Crohn's Disease

Crohn's disease (CD) is a chronic, progressive inflammatory disease of the gastrointestinal tract characterized by periods of relapse and remission, which can ultimately lead to bowel damage and disability. Most patients present with an inflammatory phenotype, but over time, uncontrolled inflammation can lead to complications such as fibrotic strictures, fistula formation, or intestinal neoplasia. Half of all patients with CD develop intestinal complications within 20 years of diagnosis leading to surgical intervention.

The current goals of CD treatment are to induce and maintain clinical and endoscopic remission, halt the progressive course of disease, and prevent long-term complications. Current available therapies such as corticosteroids, immunosuppressants, and advanced therapies, including biological agents, are effective in many patients; however, the long-term efficacy rates remain unsatisfactory, with up to 30% of patients not exhibiting an initial response to treatment, and up to 50% of patients losing response over time. Furthermore, currently available advanced therapies are associated with various risks or adverse drug reactions such as serious infections, cardiovascular events, thrombosis, and malignancies.

Despite available treatments, disease progression and complications result in an estimated 50%-80% of CD patients requiring surgery in their lifetime. While timely surgery is appropriate to avoid complications, surgery is not curative and carries risks of postoperative complications with associated risks of mortality. Furthermore, postoperative recurrence is common, depending on patient risk factors, such that approximately 50% of patients have endoscopic recurrence within one year of ileocolic resection. Repeated surgical procedures can result in short bowel syndrome with chronic malabsorption and potential dependence on total parenteral nutrition. Therefore, an unmet need exists for more safe and effective treatments for CD.

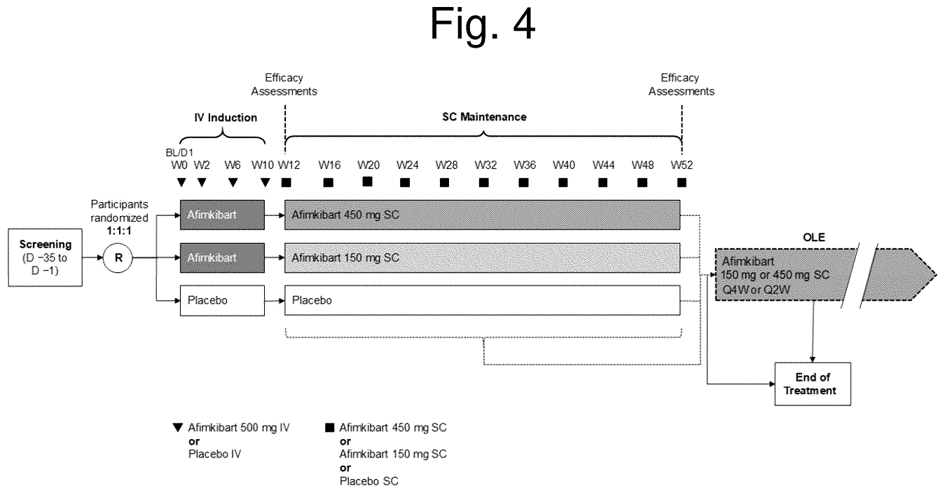

The GA45331 study provided in these Examples is a Phase 3, multicenter, double-blind, placebo-controlled, treat-through study designed to assess the efficacy, safety, pharmacokinetics, and pharmacodynamics of induction and maintenance therapy with afimkibart (formerly PF 06480605, RVT-3101, or RO7790121) compared with placebo in patients with moderately to severely active Crohn's disease (CD). A study schema for the GA45331 study is provided in FIG. 4. The maintenance and open-label extension (OLE) phases may comprise SC administration of afimkibart at a dose of 150 mg or 450 mg.

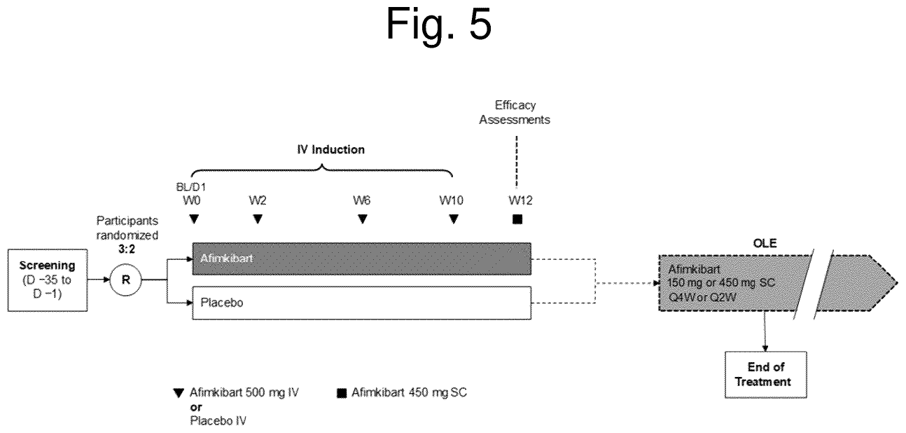

The GA45332 study provided in these Examples is a Phase 3, multicenter, double-blind, placebo-controlled study designed to assess the efficacy, safety, pharmacokinetics, and pharmacodynamics of induction therapy with afimkibart compared with placebo in patients with moderately to severely active CD. A study schema for the GA45331 study is provided in FIG. 5. The OLE phase may comprise SC administration of afimkibart at a dose of 150 mg or 450 mg.

Therapeutic options for CD have expanded substantially over the past decade, with biologics (e.g., anti-tumor necrosis factor (TNF), anti-IL-12/IL-23, and anti-integrin molecule mAbs) and small-molecule treatment (e.g., the Janus kinase (JAK) inhibitor upadacitinib) now being available in addition to conventional therapies. However, a high unmet medical need remains for treatments with better benefit-risk profiles that attenuate inflammation and clinical sequelae and provide sustained control to improve the long-term prognosis of patients with CD.

Early intervention and monitoring are crucial to prevent CD-related complications, with further evidence supporting that early initiation of advanced therapy leads to better outcomes for patients with CD (Khanna et al., Lancet, 386:1825-1834, 2015; Colombel et al., Lancet, 390:2779-2789, 2017; Ungaro et al., Aliment Pharmacol Ther, 51:831-842, 2020; Ben-Horin et al., Gastroenterology, 162:482-494, 2022; Noor et al., Lancet Gastroenterol Hepatol, 9:415-427, 2024). Accordingly, the GA45331 and GA45332 study designs aim to enroll a representative CD population to ensure that the study findings are applicable to the broader CD population in real-world clinical practice. This is achieved by including patients with moderately to severely active CD who have had inadequate response, loss of response, and/or intolerance to prior conventional therapy or advanced therapy.

### A. Primary Objectives and Corresponding Estimands

The primary objective of the GA45331 study is to evaluate the efficacy of afimkibart in maintaining clinical remission and endoscopic response compared with placebo (Table 21).

The primary objective of the GA45332 study is to evaluate the efficacy of afimkibart in inducing clinical remission and endoscopic response compared with placebo (Table 22).

TABLE 21
Primary objective of the GA45331 study

| Primary Objective | Endpoints |
|---|---|
| To evaluate the efficacy of afimkibart | Clinical remission, defined as Crohn's disease activity index |
| compared with placebo in maintaining | (CDAI) <150, at Week 52. |
| response. | Endoscopic response, defined as decrease in Simple |
| | Endoscopic Score for Crohn's disease (SES-CD) from |
| | baseline ≥50%, at Week 52. |

TABLE 22
Primary objective of the GA45332 study

| Primary Objective | Endpoints |
|---|---|
| To evaluate the efficacy of afimkibart | Clinical remission, defined as Crohn's disease activity index |
| compared with placebo in inducing | (CDAI) <150, at Week 12. |
| response. | Endoscopic response, defined as decrease in Simple |
| | Endoscopic Score for Crohn's disease (SES-CD) from |
| | baseline ≥50%, at Week 12. |

Clinical remission is defined on the basis of the Crohn's disease activity index (CDAI), and endoscopic response is defined on the basis of the Simple Endoscopic Score for Crohn's disease (SES-CD).

Statistical inference supporting this evaluation will target estimands representing the effect of assignment to the treatment conditions (an afimkibart dosing regimen vs. placebo) on a specified outcome (endpoint) in the population of patients with moderately to severely active CD, as identified by key trial inclusion and exclusion criteria.

As noted by the International Council for Harmonisation (ICH) E9 (R1) addendum on estimands and sensitivity analysis in clinical trials (ICH 2020) to the guideline on statistical principles for clinical trials, the availability or interpretation of endpoint measurements may be affected by the occurrence of intercurrent events (ICEs; ICH 2020) arising between randomization and endpoint assessment. Strategies to address anticipated ICEs are summarized in Table 23.

TABLE 23
Strategies for Anticipated Intercurrent Events

| Anticipated ICE | Strategy |
|---|---|
| Discontinuation of blinded treatment. | Composite: ICE is considered indicative of |
| Increase from baseline or initiation of | treatment failure, and the affected endpoint |
| permitted or prohibited concomitant | measurement will be imputed to a value that |
| medications to treat CD, due to lack of | is deemed unfavorable. |
| efficacy. | For the primary endpoint, this value |
| CD-related surgery with the exception of seton | corresponds to not achieving remission or |
| management for perianal fistula. | response. |
| Decrease in permitted concomitant | Treatment policy: Ignore ICE in statistical |
| medications for CD. | inference. |

CD = Crohn's disease; ICE = intercurrent event.

Under these strategies, the estimand considered in the evaluation of clinical remission at Week 52 (GA45331 study) or the estimand supporting the first co-primary endpoint (GA45332 study) amounts to a treatment effect summarized by the difference between two proportions:
the percentage of patients from the target population in clinical remission upon a successful 52-week course of therapy (GA45331 study) or a successful 12-week course of induction therapy (GA45332 study), after assignment to a particular dosing regimen of afimkibart; versus
this same percentage, had these patients been instead assigned to placebo.

The estimand for endoscopic response at Week 52 (GA45331 study) or Week 12 (GA45332 study) is defined similarly.

Primary analysis of the GA45331 study data will yield estimates for these treatment effects. Should all estimates favor at least one dosing regimen of afimkibart over placebo and be deemed statistically significant, the study will be considered positive.

Primary analysis of the GA45332 study data will yield estimates for the induction treatment effects. Should both estimates favor at least one dosing regimen of afimkibart over placebo and be deemed statistically significant, the study will be considered positive.

Efficacy is further considered in secondary objectives, provided in Table 24 (for the GA45331 study) and Table 25 (for the GA45332 study). As with the co-primary objectives, the secondary efficacy objectives have corresponding estimands of interest representing an effect comparing the same treatment conditions (afimkibart vs. placebo) on a specified endpoint, within the same overall population of patients, and using the same strategies for anticipated ICEs (Table 23).

These treatment effects are defined in the same manner as those for the primary endpoints, with effects on binary efficacy endpoints summarized by an afimkibart vs. placebo difference between proportions. For other efficacy endpoints, the effect is, generally, a difference in a summary outcome measure such as a mean score or mean change from baseline score.

TABLE 24
Key secondary objectives of the GA45331 study

| Key Secondary Objectives | Endpoints |
|---|---|
| To evaluate the efficacy of afimkibart | Clinical remission, defined as CDAI <150, at Week 12. |
| compared with placebo in inducing | Endoscopic response, defined as decrease in SES- |
| response. | CD from baseline ≥50%, at Week 12. |
| | Symptomatic remission, defined as average of |
| | daily number of liquid or very soft stools in the past |
| | week (SF) ≤2.8 and average of daily abdominal |
| | pain scores in the past week (APS) ≤1 with |
| | neither score greater than baseline, at Week 12. |
| | Endoscopic remission, defined as SES-CD = 0 to |
| | 4 with decrease from baseline ≥2 and no |
| | subscore >1, at Week 12. |
| | Ulcer-free endoscopy, defined as SES-CD ulcerated |
| | surface subscore of 0, at Week 12. |
| | SF, from baseline through Week 12. |
| | APS, from baseline through Week 12. |
| To evaluate the efficacy of afimkibart | Endoscopic remission, as defined above, at Week |
| compared with placebo in maintaining | 52. |
| response. | Symptomatic remission, as defined above, at |
| | Week 52. |
| | Corticosteroid-free clinical remission, defined as |
| | clinical remission at Week 52 and no use of |
| | corticosteroids for CD at least 8 weeks prior to Week |
| | 52. |
| | Maintenance of clinical remission, defined as clinical |
| | remission at both Weeks 12 and 52. |
| | Maintenance of endoscopic response, defined as |
| | endoscopic response at both Weeks 12 and 52. |
| | Clinical remission and endoscopic remission, |
| | as defined above, at Week 52. |
| | Ulcer-free endoscopy, as defined above, at |
| | Week 52. |
| To evaluate the efficacy of afimkibart | Bowel urgency, from baseline through Week 12 and |
| compared with placebo in terms of CD- | Week 52. |
| related symptoms and health-related | Fatigue, as measured by Functional Assessment of |
| quality of life. | Chronic Illness Therapy-Fatigue Scale (FACIT-F), |
| | from baseline to Week 12 and Week 52. |
| | Inflammatory Bowel Disease Questionnaire (IBDQ), |
| | from baseline to Week 12 and Week 52. |
| To evaluate the efficacy of afimkibart | Among TL1A biomarker-defined subgroups of |
| compared with placebo in TL1A | participants: |
| biomarker-defined subpopulations of | Clinical remission at Week 12. |
| participants. | Clinical remission at Week 52. |
| | Endoscopic response at Week 12. |
| | Endoscopic response at Week 52. |

TABLE 25
Key secondary objectives of the GA45332 study

| Key Secondary Objectives | Endpoints |
|---|---|
| To evaluate the efficacy of afimkibart | Symptomatic remission, as defined in Table 24, at |
| compared with placebo in inducing | Week 12. |
| response. | Endoscopic remission, as defined in Table 24, at |
| | Week 12. |
| | Ulcer-free endoscopy, as defined in Table 24, at |
| | Week 12. |
| | Stool frequency (SF), from baseline through Week 12. |
| | Abdominal pain score (APS), from baseline through |
| | Week 12. |
| To evaluate the efficacy of afimkibart | Bowel urgency, from baseline through Week 12. |
| compared with placebo in terms of CD- | Fatigue, as measured by FACIT-F, from |
| related symptoms and health-related | baseline to Week 12. |
| quality of life. | IBDQ score, from baseline to Week 12. |
| To evaluate the efficacy of afimkibart | Among TL1A biomarker-defined subgroups of |
| compared with placebo in TL1A | participants: |
| biomarker-defined subpopulations of | Clinical remission at Week 12. |
| participants. | Endoscopic response at Week 12. |

The TL1A biomarker is TNFSF15 haplotype B carrier status (haplotype B carrier vs. haplotype B non-carrier). See Example 1, above.

Other secondary objectives of the studies (Table 26) also consider both efficacy and safety. Safety endpoints refer to a summary outcome measure, rather than a participant-level outcome variable.

TABLE 26
Other secondary objectives of the GA45331 and GA45332 studies

| Other Secondary Objectives | Endpoints |
|---|---|
| To evaluate the efficacy of afimkibart | Clinical response, defined as a decrease in CDAI |
| compared with placebo in inducing (both | from baseline ≥100, at Week 12. |
| studies) and/or maintaining (GA45331 | Symptomatic response, defined as decrease in |
| study) response. | SF and APS ≥30% with neither greater than |
| | baseline, at Week 12. |
| To evaluate the efficacy of afimkibart | Overall change in CD symptoms, as measured by |
| compared with placebo in terms of the | Patient Global Impression of Change (PGIC), |
| participant's global impressions and | from baseline to Weeks 2, 6, and 12 (GA45332 |
| general well-being. | study) or Weeks 2, 6, 12, and 52 (GA45331 |
| | study). |
| | Overall severity in CD symptoms, as measured by |
| | Patient Global Impression of Severity (PGIS), |
| | from baseline to Weeks 2, 6, and 12 (GA45332 |
| | study) or Weeks 2, 6, 12, and 52 (GA45331 |
| | study). |
| | General well-being, from baseline through Week 12 |
| | (GA45332 study) or Week 52 (GA45331 study). |
| To evaluate the safety of afimkibart | Incidence and severity of the following: |
| compared with placebo. | Adverse events. |
| | Serious adverse events. |
| | Adverse events leading to study treatment |
| | discontinuation. |
| | Adverse events of special interest. |
| To evaluate the persistence of fistulas of | Presence of draining fistulas from baseline through |
| participants treated with afimkibart | Week 12 (GA45332 study) or Week 12 and Week |
| compared to placebo. | 52 (GA45331 study). |

Exploratory objectives and corresponding endpoints for the GA45331 and GA45332 studies are described below. Estimands for exploratory efficacy objectives are defined in a similar manner to those supporting primary and secondary objectives. As with safety domains, exploratory objectives beyond efficacy may have endpoints that describe a summary outcome measure rather than a participant-level outcome variable.

TABLE 27
Exploratory objectives of the GA45331 and GA45332 studies

| Exploratory Objectives | Endpoints |
|---|---|
| To evaluate the efficacy of | Change in CDAI from baseline through Week 12 (GA45332 |
| afimkibart compared with | study) or Week 52 (GA45331 study). |
| placebo in inducing and/or | Change in SES-CD from baseline to Week 12 (GA45332 |
| maintaining response. | study) or Week 12 and Week 52 (GA45331 study). |
| | Change in Geboes Grading Scale from baseline to Week 12 |
| | (GA45332 study) or Week 12 and Week 52 (GA45331 study). |
| To characterize the | Pre-dose and peak concentration of afimkibart in serum at |
| pharmacokinetics of afimkibart. | specified timepoints. |
| To evaluate potential effects of | ADA and NAb detection in serum at specified timepoints. |
| immunogenicity of afimkibart. | Association of ADA and NAb status (or titers) with efficacy, |
| | safety, PK, or PD endpoints. |
| To evaluate the | Total soluble TL1A concentration in serum at specified |
| pharmacodynamics of | timepoints. |
| afimkibart. | Association of these levels with PK, immunogenicity, and |
| | disease activity. |
| To evaluate the health utility of | EQ-5D-5L index-based and visual analog scale (VAS) scores |
| participants treated with | from baseline to Week 12 (GA45332 study) or Week 12 and |
| afimkibart compared with | Week 52 (GA45331 study). |
| placebo. | WPAI:CD score from baseline to Week 12 (GA45332 study) or |
| | Week 12 and Week 52 (GA45331 study). |
| To evaluate biomarkers in | Fecal calprotectin at specified timepoints. |
| participants treated with | C-reactive protein at specified timepoints. |
| afimkibart compared with | Pathway and pathophysiology biomarker levels from baseline |
| placebo. | through Week 12 (GA45332 study) or Week 52 (GA45331 |
| | study), and associations of these levels amongst each other and |
| | with PK, PD, and disease activity. |
| To evaluate automated | Artificial intelligence (AI)-based assessment of endoscopic |
| endoscopy assessments in | activity from baseline to Week 12 (GA45332 study) or Week 12 |
| participants treated with | and Week 52 (GA45331 study). |
| afimkibart or placebo. | AI-based spatial measurements of mucosal features, from |
| | baseline to Week 12 (GA45332 study) or Week 12 and Week 52 |
| | (GA45331 study). |
| | Association of these assessments with treatment arm and with |
| | disease activity. |

### Example 8. CD Study Design

### A. Overall Design

The GA45331 and GA45332 study populations include participants with moderately to severely active CD for whom at least one of the following prior therapy types have failed:
Conventional therapy (aminosalicylates, corticosteroids, and/or immunosuppressants).
Advanced therapy (includes biologics or targeted small molecules, e.g., an anti-TNF agent, an anti-IL-12/IL-23 agent, an anti-integrin agent, a JAK inhibitor, etc.).

Approximately 600 participants are enrolled in the GA45331 study across global investigational sites. Approximately 425 participants are enrolled in the GA45332 study across global investigational sites. Each study has balanced representation of participants who have demonstrated conventional or advanced therapy failure. Enrollment of participants who have failed three or more advanced therapies (i.e., number of therapies, not classes of therapies) is capped to at most 30%.

The GA45331 study has a treat-through design that consists of a screening period of up to 35 days (+7 days) to determine eligibility; a 12-week induction treatment phase; a 40-week maintenance treatment phase; an optional open-label extension (OLE) treatment phase; and a safety follow-up period of 12 weeks (consisting of two visits, one at 6 weeks and one at 12 weeks) following the final dose of study treatment.

The GA45332 study has a design that consists of a screening period of up to 35 days (+7 days) to determine eligibility; a 12-week induction treatment phase; an optional open-label extension (OLE) treatment phase; and a safety follow-up period of 12 weeks (consisting of two visits, one at 6 weeks and one at 12 weeks) following the final dose of study treatment.

Entry criteria for both studies are based on confirmation of moderately to severely active CD during screening, defined as a CDAI score of ≥220 and ≤450, and SES-CD of ≥6 (or ≥4 for isolated ileal disease only) at baseline.

In the GA45331 study, eligible study participants are randomly assigned to one of the following treatment arms:
Afimkibart:
   (1) 500 mg intravenously (IV) at Weeks 0, 2, 6, and 10, followed by 450 mg subcutaneously (SC) Q4W from Week 12 through Week 52, or
   (2) 500 mg IV at Weeks 0, 2, 6, and 10, followed by 150 mg SC Q4W from Week 12 through Week 52.
Placebo: placebo IV at Weeks 0, 2, 6, and 10, followed by placebo SC Q4W from Week 12 through Week 52.

In the GA45332 study, eligible study participants are randomly assigned to one of the following treatment arms:
Afimkibart: 500 mg intravenously (IV) at Weeks 0, 2, 6, and 10.
Placebo: placebo IV at Weeks 0, 2, 6, and 10.

In the GA45331 study, randomization is performed according to a 1:1:1 allocation ratio. In the GA45332 study, randomization is performed according to a 3:2 allocation ratio. Randomization is stratified by:
(a) Prior advanced therapy use (yes/no),
(b) Baseline corticosteroid use (yes/no),
(c) Baseline endoscopic activity (moderate (SES-CD≤15) or severe (SES-CD>15)); and
(d) Baseline Crohn's disease activity index (CDAI)≤330 (yes/no).

In the GA45331 study, the induction phase (IV dosing of afimkibart or matching placebo at Weeks 0-10) evaluates the induction of clinical remission and endoscopic response at Week 12. After completion of the induction phase, participants continue with the administration of afimkibart or matching placebo during the maintenance phase (SC dosing at Weeks 12-52), in which the durability of the clinical remission and endoscopic response are examined. All participants who have completed Week 12, including those randomized to the placebo arm, are eligible to enter the optional open-label extension (OLE) phase and receive active treatment with 150 mg or 450 mg SC administration of afimkibart (at the dose administered during the maintenance phase) provided that they meet all OLE phase eligibility criteria. Disease worsening continues to be monitored by the investigator after Week 12. Participants who complete all trial procedures, including study treatment administration at Week 52, or who meet the disease worsening criteria at any time during the maintenance phase will have the option to continue to the OLE phase and receive active treatment.

In the GA45332 study, the induction phase (IV dosing of afimkibart or matching placebo at Weeks 0-10) evaluates the induction of clinical remission and endoscopic response at Week 12. Participants who have completed Week 12 assessments are eligible to enter the optional OLE phase and receive active treatment with 150 mg or 450 mg SC administration of afimkibart provided that they meet all OLE eligibility criteria. Disease worsening criteria are assessed during the OLE. Participants who do not complete the 12-week induction phase for any reason are not eligible for the OLE.

Participants are assessed for efficacy by using a Crohn's disease activity index (CDAI) score and undergo a full endoscopy (ileocolonoscopy) with biopsy to determine Simple Endoscopic Score for Crohn's disease (SES-CD) at screening, Week 12, and Week 52 (for the GA45331 study) or at screening and Week 12 (for the GA45332 study). The CDAI score is a measure of CD disease activity and is a composite of eight assessments: number of liquid or soft stools (SF), abdominal pain (APS), general well-being, presence of complications, use of anti-diarrheals (e.g., LOMOTIL® (diphenoxylate/atropine) or other opiates for diarrhea), presence of an abdominal mass, hematocrit, and percentage deviation from standard weight. SES-CD is based on the centrally-read endoscopy and a composite of four assessments, each rated from 0 to 3: size of ulcers, proportion of the surface covered by ulcers, proportion of the surface with any other lesions, and presence of narrowing (stenosis) across five anatomic locations (terminal ileum, right colon, transverse colon, sigmoid and left colon, and rectum).

The patient-reported outcome (PRO) components of the CDAI (number of liquid or soft stools, abdominal pain, and general well-being) and patient-reported CD signs and symptoms, such as bowel urgency, are collected daily during the screening, induction, and maintenance phases, using an electronic diary (eDiary). During the OLE phase, participants complete eDiary PROs daily for at least 7 days prior to each Q3M (every three months) study visit during the first year, and prior to annual study visits thereafter.

If disease worsening criteria assessment is required in the OLE phase, eDiary PROs are collected for at least 7 days prior to the study visit at which the assessment is conducted.

### B. Open-Label Extension Phase

All participants in the GA45331 and GA45332 studies have the opportunity to participate in the optional open-label extension (OLE) phase of the study with access to afimkibart and monitoring, irrespective of whether they were previously randomized to receive afimkibart or placebo during the double-blind phase, provided they meet specified eligibility criteria.

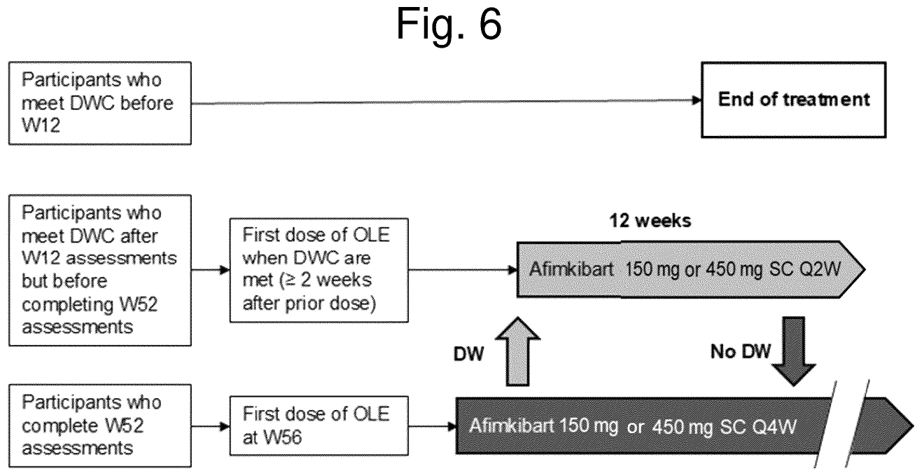

A study schema for the OLE phase of the GA45331 study is provided in FIG. 6. The OLE phase may comprise SC administration at a dose of 150 mg or 450 mg (Q2W or Q4W).

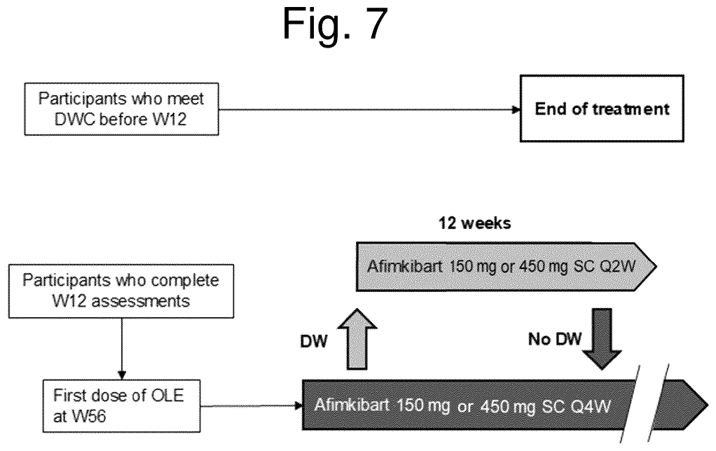

A study schema for the OLE phase of the GA45332 study is provided in FIG. 7. The OLE phase may comprise SC administration at a dose of 150 mg or 450 mg (Q2W or Q4W).

In the GA45331 study, participants may be eligible to enter the OLE phase at the following time points:
At any time after completion of the 12-week induction phase and upon completion of the Week 12 assessments and first dose of maintenance, if the participant meets disease worsening criteria. Participants are not eligible for the OLE phase before Week 12.
After completion of the 40-week maintenance phase and upon completion of the Week 52 assessments.

In the GA45332 study, participants may be eligible to enter the OLE phase any time after completion of the 12-week induction phase and upon completion of the Week 12 assessments.

In the GA45331 study, starting after the Week 12 assessments and the first dose of maintenance therapy, participants who, based on an assessment by the investigator, have not improved or have worsened compared with baseline (Day 1 of Week 0), may be eligible to enroll in the OLE, provided they meet both of the symptomatic and endoscopic criteria provided below (disease worsening criteria).

### Disease Worsening Criteria:

1. On at least two consecutive visits (scheduled or unscheduled) between 2 and 4 weeks apart (and within the scheduled visit window), a CDAI assessment fulfilling both: 1a. CDAI ≥220; and
1b. <70-point decrease in CDAI from baseline;
and
2. A centrally-read endoscopy indicating moderate-to-severe endoscopic activity with no meaningful improvement; fulfilling both:
2a. SES-CD ≥6 (or >4 for isolated ileal disease), and
2b. <3-point decrease (or <1-point decrease for isolated ileal disease at baseline) from baseline in SES-CD.

The thresholds on post-baseline CDAI and SES-CD in criteria 1a and 2a, respectively, clinically identify moderately-to-severely active CD patients and are consistent with the study population inclusion criteria (Example 9). The thresholds on decrease from baseline in CDAI and SES-CD in criteria 1b and 2b, respectively, correspond to values that would generally not be considered a clinically meaningful change, provided that the post-baseline score remains moderate to severe. Therefore, the above criteria enable the identification of participants who have not demonstrated clinically meaningful improvement from baseline moderate-to-severe disease activity and thus are eligible for rollover to the open-label extension phase.

In the assessment of disease worsening to determine changes to the OLE dose schedule, the investigator may use their discretion in the need for unscheduled endoscopies during the OLE phase. Should the investigator decide to forgo such an endoscopy in the assessment of disease worsening, it is permissible to apply the symptomatic criteria (1a and 1b above) in conjunction with an alternative objective criterion of fecal calprotectin ≥150 mg/g per the American Gastroenterological Association Guidelines (Ananthakrishnan et al., Gastroenterology, 165:1367-1399, 2023). Fulfillment of disease worsening criteria is captured by efficacy assessments. In the evaluation of disease worsening, potential differential diagnosis, such as the presence of enteric pathogens, is ruled out by stool culture/sensitivity, ova and parasite evaluation including C. difficile testing.

i. Open-Label Extension Phase Dose Schedule in the GA45331 Study

In the GA45331 study, during the maintenance phase (after Week 12), if the participant's condition meets the disease worsening criteria, the participant may either discontinue blinded treatment and enter the OLE phase using an every 2 weeks (Q2W) dose intensification schedule (150 mg or 450 mg SC Q2W) for 12 weeks or be withdrawn from the study.

Participants who follow the 150 mg Q2W dose schedule will have the opportunity of de-escalating to the 150 mg SC Q4W dose schedule after 12 weeks or being withdrawn from the study.

Participants who follow the 450 mg Q2W dose schedule will have the opportunity of de-escalating to the 450 mg SC Q4W dose schedule after 12 weeks or being withdrawn from the study.

Participants who complete Week 52 at the 150 mg dose and roll over into the OLE phase continue with the 150 mg Q4W dose schedule (150 mg SC Q4W). Participants who complete Week 52 at the 450 mg dose and roll over into the OLE phase continue with the 450 mg Q4W dose schedule (450 mg SC Q4W). Participants who complete Week 52 and roll over into the OLE phase continue with the Q4W dose schedule (450 mg SC Q4W). The first OLE visit for such participants is at Week 56, and the participants continue in the OLE phase on the Q4W dose regimen until the end of the study. However, if at any point during the OLE phase the participant's condition meets the disease worsening criteria, the option for dose intensification to 150 mg SC Q2W (for patients receiving the 150 mg SC Q4W dose schedule) or 450 mg SC Q2W (for patients receiving the 450 mg SC Q4W dose schedule) or withdrawal from the study is assessed. Participants who follow the 150 mg SC Q2W dose schedule have the opportunity of de-escalating to the 150 mg SC Q4W dose schedule after 12 weeks or may be withdrawn from the study. Participants who follow the 450 mg SC Q2W dose schedule have the opportunity of de-escalating to the 450 mg SC Q4W dose schedule after 12 weeks or may be withdrawn from the study. Doses of afimkibart can be administered at least 7 days apart; however, every effort should be made to maintain the original dosing schedule. A maximum of 2 dose intensifications to Q2W is allowed in the OLE phase of the study.

ii. Open-Label Extension Phase Dose Schedule in the GA45332 Study

In the GA45332 study, participants who complete the 12-week induction phase and roll over into the OLE phase initiate the 150 mg Q4W dose schedule (150 mg SC Q4W) or the 450 mg Q4W dose schedule (450 mg SC Q4W). The first OLE dose is at Week 12, and participants continue in the OLE phase on the Q4W dose regimen until the end of the study. However, if at any point during the OLE phase the participant's condition meets the disease worsening criteria defined above, the option for dose intensification to 150 mg SC Q2W (for patients receiving the 150 mg SC Q4W dose schedule) or 450 mg SC Q2W (for patients receiving the 450 mg SC Q4W dose schedule) or withdrawal from the study will be assessed by the investigator. Participants who follow the 150 mg SC Q2W dose schedule have the opportunity of de-escalating to the 150 mg SC Q4W dose schedule after 12 weeks or may be withdrawn from the study. Participants who follow the 450 mg Q2W dose schedule have the opportunity of de-escalating to the 450 mg SC Q4W dose schedule after 12 weeks or being withdrawn from the study. Doses can be administered at least 7 days apart; however, every effort should be made to maintain the original dosing schedule. A maximum of 2 dose intensifications to Q2W is allowed in the OLE phase of the study.

### C. End of Study Definition and Duration of Participation

A participant is considered to have completed the GA45331 or GA45332 study if he or she has completed all phases of the study, including the last visit shown in the schedules of activities.

The end of the study is defined as the date of the last visit of the last participant in the study or the date at which the last data point required for statistical analysis, safety follow-up, or statistical analysis follow-up is received from the last participant, whichever occurs later.

The end of the study is expected to occur approximately 30 weeks (for the GA45332 study) or approximately 70 weeks (for the GA45331 study) after the last participant is enrolled, unless at least one participant enters the OLE phase. For both studies, with OLE participation, treatment will continue for approximately 5 years after the last participant is enrolled in the study or until afimkibart is commercially available in that region.

### Example 9. CD Study Population

Approximately 600 participants with CD are enrolled during the global enrollment phase of the GA45331 study. Approximately 425 participants with CD are enrolled during the global enrollment phase of the GA45332 study.

### A. Inclusion Criteria

Potential participants are eligible to be included in the studies only if all of the following criteria apply:

i. General Inclusion Criteria
Age ≥18 to ≤80 years. Patients aged ≥16 to <18 years may be eligible to participate in the study where locally permissible (e.g., if permitted by local guidelines and regulations).
Body weight ≥40 kg.
   ii. Crohn's Disease-Specific Inclusion Criteria
Confirmed diagnosis of CD with supportive clinical, endoscopic and histopathological evidence.
Moderately to severely active CD, meeting all of the following:
   Simple Endoscopic Score for Crohn's Disease (SES-CD) of ≥6 (or ≥4 for isolated ileal disease) confirmed through a centrally read endoscopy performed either (i) during the screening period or (ii) before the screening period (independently of the study), within 4 weeks of randomization, and in patients who already have an established CD diagnosis.
   AND
   Crohn's disease activity index (CDAI) score of ≥220 and ≤450.
Involvement of ileum and/or colon, with at least four colonic segments traversable by an endoscope or a pediatric endoscope, or three segments for patients who have undergone a bowel resection among the following segments: terminal ileum, right colon, transverse colon, sigmoid and left colon, and rectum.
Screening for colorectal cancer (CRC) during or prior to screening for all participants (performed according to local standards).
   For participants with colonic disease lasting for ≥8 years' duration or with risk factors for bowel cancer, a surveillance ileocolonoscopy must be performed within 12 months prior to screening. For all other patients, must be up-to-date with CRC surveillance (according to CRC risks and local standards). Screening ileocolonoscopy can be used for CRC surveillance (following local guidelines) and results must be available prior to randomization.
   Any adenomatous polyps must be completely removed according to routine practice prior to their first dose of study drug.
      iii. Reproductive Inclusion Criteria.
For female participants of childbearing potential: agreement to remain abstinent (refrain from heterosexual intercourse) or use an acceptable method of contraception and agree to refrain from egg donation or undergoing fertility treatment during the treatment period and for 95 days after the final dose of afimkibart.
For male participants: agreement to remain abstinent (refrain from heterosexual intercourse) or use a condom, and agree to refrain from donating sperm.
   iv. Prior Medication Inclusion Criteria.

Must have had at least one of the following treatments in the past with inadequate response, loss of response, and/or intolerance. Inadequate response is defined as having signs and symptoms of persistently active disease despite completing at least the approved dosing regimen in the product label. Intolerance may include, but is not limited to, infusion-related reactions, injection site reactions, rash, serum sickness, hepatic abnormalities, demyelination, congestive heart failure, and infections. There is no minimum requirement for dose or duration if a potential participant was determined to be intolerant to prior treatment. Loss or response is defined as the recurrence of signs and symptoms of active disease during approved treatment following prior clinical benefit (discontinuation despite clinical benefit does not qualify as having failed or being intolerant to CD advanced therapy). Participants previously exposed to investigational therapies for the treatment of CD must still meet inclusion criteria “Conventional Therapy Failure” or “Advanced Therapy Failure.”

### Conventional Therapy Failure:

Steroids (e.g., systemic prednisone, oral budesonide). The following definitions will be used as guidelines for the use of corticosteroids in this trial:
Corticosteroid refractory: Persistent active disease despite treatment with at least one 4-week induction regimen, including a starting dose of ≥30 mg of oral prednisone (or equivalent) for at least 2 weeks or IV prednisone for ≥5 days, or persistently active disease after at least 4 weeks of oral budesonide given 9 mg/day.
Corticosteroid dependent: Unable to reduce corticosteroids below the equivalent of prednisone 10 mg/day or prednisolone 10 mg/day within 3 months of starting steroids, without recurrent active disease, or relapse within 3 months of stopping steroids.
Corticosteroid intolerant: History of intolerance to corticosteroids (including but not limited to Cushing syndrome, osteopenia/osteoporosis, hyperglycemia, insomnia, infection).
At least 12 weeks of an immunomodulator, which can include:
   ≥1.5 mg/kg/day of oral azathioprine (AZA) (or per local standard of care).
   ≥0.75 mg/kg/day of oral 6-mercaptopurine (MP) (or per local standard of care).
   ≥15 mg/week of intramuscular or subcutaneous (SC) methotrexate (MTX).
   Persistent signs and symptoms of active disease despite a 6-TG level of ≥230 pmol/8×10⁸ RBCs during at least one 12-week regimen of oral AZA or 6-MP at a stable or increasing dose.
   History of intolerance to AZA, 6-MP, or methotrexate (including, but not limited to, nausea/vomiting, abdominal pain, pancreatitis, LFT abnormalities, lymphopenia, TPMT genetic mutation, infection).
At least 4 weeks of an oral aminosalicylate, which can include a minimum dose of the following:
   2.4 g/day mesalamine (or per local standard of care).
   4 g/day sulfasalazine (or per local standard of care).
   1 g/day olsalazine (or per local standard of care).
   6.75 g/day balsalazide (or per local standard of care).

### Advanced Therapy Failure:

The medications used to qualify the participant for entry into this category include the following therapies (and any relevant biosimilars) approved for the treatment of CD.
Approved anti-TNF agents, including and not limited to the following:
   At least one 6-week induction regimen of infliximab (≥5 mg/kg IV at 0, 2, and 6 weeks or per local label) or equivalent biosimilar.
   At least one 8-week induction regimen of adalimumab (one 160 mg SC dose followed by one 80 mg SC dose (or one 80 mg SC dose in countries where this dosing regimen is allowed), followed by one 40 mg SC dose at least 2 weeks apart or per local label) or equivalent biosimilar.
Approved anti-integrins, including and not limited to the following:
   At least one 6-week induction regimen of vedolizumab (300 mg IV at 0, 2, and 6 weeks or per local label).
Approved anti-IL-12/IL-23, including and not limited to the following:
   At least one 8-week induction regimen of ustekinumab (a single IV dose using weight-based dosing: 260 mg for participants with body weight ≤55 kg; 390 mg for participants with body weight >55 kg to ≤85 kg; 520 mg for participants with body weight >85 kg or per local label) or equivalent biosimilar.
   At least one 8-week induction regimen of risankizumab (600 mg IV at 0, 4, and 8 weeks, or per local label).
At least one 8-week regimen of mirikizumab (900 mg IV at Weeks 0, 4, and 8 or per local label).
At least one 8-week induction regimen of guselkumab (200 mg IV at Weeks 0, 4, and 8, or 400 mg SC at Weeks 0, 4, and 8, or per local label).
Approved JAK inhibitors, including and not limited to the following:
   At least one 8-week induction course of upadacitinib (45 mg orally daily or per local label).
Any newly approved sphingosine-1-phosphate (S1P) receptor modulators for CD treatment.

The “Advanced Therapy Failure” inclusion criteria may also be fulfilled with exposure to investigational advanced therapy, provided that all of the following are met:
Exposure to the investigational advanced therapy has been confirmed (i.e. the patient has been unblinded).
The investigational advanced therapy has been approved for the treatment of CD. This approval need not be local.
The patient completed and failed at least the equivalent of the approved induction dosing regimen, per the investigator's discretion.

### B. Exclusion Criteria

Potential participants are excluded from the studies if any of the following criteria apply:

i. Inflammatory Bowel Disease Exclusion Criteria.
Participant with a history of ≥3 bowel resections.
   >2 missing segments of the following 5 segments: terminal ileum, right colon, transverse colon, sigmoid and left colon, and rectum.
Diagnosis of short gut or short bowel syndrome.
Presence of ileostomy, colostomy, or ileo-anal pouch.
Patients with symptomatic bowel strictures, fulminant colitis, or toxic megacolon.
Current diagnosis of UC or indeterminate colitis, ischemic colitis, infectious colitis, radiation colitis, or microscopic colitis.
Presence of abdominal or perianal abscess.
Presence of rectovaginal, enterovaginal, or high output enterocutaneous fistula, enterovesical fistulas, or perianal fistulas with >3 openings and/or the anticipated need for surgery during the study (except surgery for seton placement and/or removal).
Current diagnosis or suspicion of primary sclerosing cholangitis.
   ii. Medical History Exclusion Criteria.
Lack of peripheral venous access.
Any major surgery within 6 weeks prior to screening or a major surgery planned during the study.
Significant uncontrolled medical comorbidity (such as cardiac, pulmonary, renal, hepatic, endocrine, or gastrointestinal disorders (excluding CD)), psychiatric condition, or other condition that, in the opinion of the investigator, would confound the study results, compromise patient safety, or interfere with the potential participant's provision of informed consent or compliance with trial procedures.
Pregnancy or breastfeeding, or intention of becoming pregnant during the study or within 95 days after the final dose of afimkibart.
Any condition that would preclude endoscopic evaluation.
Any past or current evidence of cancer of the gastrointestinal tract or definite low-grade or high-grade colonic dysplasia. Adenomas or any other benign neoplasia that are not completely removed are exclusionary. Following complete removal, the participant may be eligible for the study.
History of non-gastrointestinal cancer within 5 years prior to screening visit (including, e.g., solid tumors, hematologic malignancies, etc.), with the exception of adequately treated non-metastatic basal cell or squamous cell skin cancer or in situ cervical cancer.
History of alcohol, drug, or chemical abuse <1 year prior to screening.
History of blood transfusion within 30 days prior to screening.
   iii. Infection or Infection Risk Exclusion Criteria.
Any clinically significant infection <4 weeks prior to randomization that has not resolved, and/or that required hospitalization and/or IV antibiotics. Any clinically significant infection that was opportunistic in nature is not permitted within 3 months prior to randomization.
Evidence of or treatment for Clostridioides difficile (C. difficile; formerly known as Clostridium difficile) as assessed by detection of C. difficile toxin within 30 days prior to randomization (Week 0, Day 1), or other intestinal pathogens (as assessed by stool culture/sensitivity and ova and parasite evaluation) within 30 days prior to randomization (Week 0, Day 1).
Any diagnosis of cytomegalovirus (CMV) colitis in the past 30 days (including diagnosis during screening).
Confirmation of HIV infection (e.g., positive HIV test) at screening.
Positive test results for hepatitis B infection at screening, defined as meeting either of the following criteria: (a) Positive hepatitis B surface antigen (HBsAg) test at screening; or (b) Negative HBsAg test and positive total hepatitis B core antibody (HBcAb) test in combination with quantitative HBV DNA above the lower limit of quantification.
Positive hepatitis C virus antibody test during screening, with the following exception: Participants with positive HCV antibody test and quantitative HCV RNA levels below the lower limit of quantification, confirmed by RNA PCR test, are not excluded provided they have completed a successful course of HCV anti-viral treatment with no HCV recurrence for ≥6 months.
Participants with latent tuberculosis (TB) or potentially active TB as defined by a positive QUANTIFERON®-TB Gold (QFT) test result, a positive purified protein derivative (PPD) skin test, or other locally approved TB enzyme-linked immunosorbent assay (ELISA) test (e.g., T-SPOT®) must agree to comply with testing and management.
Active tuberculosis (TB) infection suggested by positive TB testing, clinical symptoms, and/or chest imaging (X-ray or CT).
History of organ transplant.
Acquired or congenital immunodeficiency.
   iv. Laboratory Results Exclusion Criteria.
Clinically significant abnormality on laboratory tests during screening (hematology, serum chemistry, and urinalysis) that, in the opinion of the investigator, may pose an additional risk in administering study treatment to the potential participant.
ALT, AST, or ALP ≥2.5× upper limit of normal, total bilirubin ≥2× upper limit of normal, or presence of abnormalities in synthetic liver function tests judged to be clinically significant by the investigator. Patients with an established diagnosis of Gilbert's syndrome (i.e., with required source documentation showing unconjugated hyperbilirubinemia with no evidence of hemolysis) with total bilirubin levels <3× ULN can be included.
ANC <1.5×10⁹/L (1500/μL) with one exception: Participants with documented benign ethnic neutropenia (BEN): ANC <1.3×10⁹/L (1300/μL).
Platelet count <100,000/μL.
Hemoglobin <8 g/dL.
Absolute lymphocyte count <500/μL.
Estimated glomerular filtration rate (eGFR)<30 mL/min/1.73 m².
   v. Prohibited Medications Exclusion Criteria

Any of the following related to previous or current treatment:
Use of approved CD treatments including approved oral small molecule (e.g., JAK inhibitor) treatments within 2 weeks prior to screening endoscopy (or SOC endoscopy), or approved biologic agents within 8 weeks or 5 half-lives prior to randomization (Week 0, Day 1), whichever is shorter. If there is proper documentation of undetectable drug level measured by a commercially available assay for any of the approved biologics, there is no minimum washout.
Use of any investigational or experimental therapy within approximately 30 days for non-biologic therapy or 8 weeks for biologic therapy, OR 5 half-lives (whichever is shorter), prior to randomization (Week 0, Day 1).
Oral prednisone >20 mg/day (or dose equivalent of other oral corticosteroids) from two weeks prior to screening endoscopy (or SOC endoscopy), or intent to receive during the study with the exception for the treatment of an adverse event.
Any immunosuppressive therapy including B-cell depleting agents (e.g., natalizumab, rituximab) or other lymphocyte depleting agents (e.g., alemtuzumab), or any other immunosuppressive agents (e.g., those where live vaccines are contraindicated) within 1 year prior to screening or intent to receive during the study.
Treatment with IV corticosteroids ≤2 weeks prior to screening endoscopy (or SOC endoscopy) or intent to receive during the study, with the exception of a single administration of IV steroid for potential infusion-related reaction (IRR) management.
Presence of conditions other than CD (e.g., uncontrolled asthma) that could require treatment with >20 mg/day of prednisone (or equivalent) during the course of the study.
Treatment with corticosteroid enemas or suppositories and/or topical (rectal) 5-ASA preparations ≤2 weeks prior to screening endoscopy (or SOC endoscopy) or intent to receive during the study.
Treatment with topical rectal traditional medicine (e.g., Chinese medicine), herbal enemas, or suppositories ≤2 weeks prior to screening endoscopy (or SOC endoscopy) or intent to receive during the study.
Treatment with approved CD oral traditional medicine (e.g., Chinese medicine) ≤4 weeks prior to screening endoscopy (or SOC endoscopy) or intent to receive during the study.

Transplant/stem cell therapy at any time prior to randomization (Week 0, Day 1) or intent to receive during the study.
Treatment ≤16 weeks prior to randomization (Week, 0, Day 1) with cyclosporine, thalidomide, tacrolimus, sirolimus, or mycophenolate mofetil or intent to receive during the study.
Apheresis ≤2 weeks prior to screening endoscopy (or SOC endoscopy) or intent to receive during the study.
Currently receiving total parenteral nutrition.
Continued tube feeding and/or total parenteral alimentation/nutrition as treatment for CD ≤3 weeks prior to randomization or during the study.
Receipt of fecal microbial transplantation ≤4 weeks prior to randomization (Week 0, Day 1).
Current or prior use of anti-TL1A antibody (afimkibart RO7790121/RVT-3101/PF-06480605) or any type of anti-TL1A therapy.
Receipt of a live or attenuated vaccine ≤4 weeks prior to screening endoscopy (or SOC endoscopy) or intent to receive during the study; use of non-live (inactivated) vaccines is allowed.
Chronic (e.g., >7 days) non-steroidal anti-inflammatory drug (NSAID) use; occasional use of NSAIDs and acetaminophen is permitted (e.g., headache, arthritis, myalgias, or menstrual cramps). Aspirin ≤325 mg/day is permitted.
Treatment with immunoglobulin or blood products within 4 weeks prior to screening, or any condition that is likely to require such treatment during the course of the study.
IV antibiotics ≤4 weeks prior to randomization (Week 0, Day 1).
Previous severe allergic reaction (NCI CTCAE v5.0 Grade 3 or higher) or anaphylactic reaction to biologic agents or to any excipients of the study drug.

### Example 10. CD Study Treatment, Other Treatments Relevant to Study Design, and Concomitant Therapy

Table 28 provides a description of assigned study treatments for the GA45331 and GA45332 studies.

TABLE 28
Study Treatment Description

| | Afimkibart | Placebo |
|---|---|---|
| Use | Investigational | Placebo comparator |
| Drug form | Solution for infusion/injection | Solution for infusion/injection |
| Unit dose strength | 225 mg/1.5 mL (150 mg/mL) | Not applicable |
| Dosage levels | Induction: 500 mg IV at Weeks 0, | Not applicable |
| | 2, 6, and 10. | |
| | Maintenance (for the GA45331 | |
| | study only): 150 mg or 450 mg SC | |
| | Q4W. | |
| | OLE: 150 mg or 450 mg SC Q2W | |
| | or Q4W. | |
| Route of | Intravenous infusion or | Intravenous infusion or |
| administration | subcutaneous injection | subcutaneous injection |

OLE = open-label extension; Q2W = every 2 weeks; Q4W = every 4 weeks.

At applicable sites, SC study treatment after Week 12 may be administered by a trained nursing professional at the participant's home or another suitable location.

Intravenous administration of afimkibart is performed in a monitored setting where there is immediate access to trained personnel and adequate equipment and medicine to manage potentially serious reactions.

The IV infusion is delivered over 60 (±10) minutes. The post-dose observation period for IV infusions is 60 minutes.

For participants who experience an infusion-related reaction (IRR), subsequent infusions may be given in accordance with institutional/local clinical guidelines or IRR management guidance.

The post-dose observation period for subcutaneous (SC) injection is 30 minutes, with the exception of the first four doses in the open-label extension (OLE) phase. These first four OLE doses, which are administered at the study site, have a post-dose observation period of 60 minutes.

### A. Concomitant Therapy

Permitted and prohibited concomitant therapies for CD are outlined in Table 29. Permitted and prohibited concomitant therapies not related to CD are outlined in Table 30. Concomitant medication restrictions apply to all phases of the studies. However, during the OLE phase, oral or rectal aminosalicylates, immunomodulators, CD-related antibiotics, oral or rectal corticosteroids for treatment of CD may be initiated, stopped, or the dose may be adjusted at the investigator's discretion. If oral corticosteroids are initiated, tapering should begin within 2 weeks. If the participant requires the same dose of corticosteroids for >2 weeks (e.g., not able to taper off), the investigator should consider discontinuing the participant from the study.

In general, investigators may manage a participant's care (including pre-existing conditions) through use of supportive therapies, as clinically indicated and per local standard practice, with the exception of prohibited therapies defined below.

TABLE 29
Permitted and Prohibited Concomitant Therapies for Crohn's Disease

| Therapy | Permitted | Prohibited |
|---|---|---|
| Anti- | Oral 5-ASA if on stable dose for ≥2 | Oral 5-ASA initiation or dose change |
| inflammatories | weeks prior to screening endoscopy (or | throughout the duration of the study. |
| | SOC endoscopy); stable dose throughout | Topical (rectal) 5-ASA ≤2 weeks prior |
| | the study is permitted unless it is causing | to screening endoscopy (or SOC |
| | toxicity, in which case the dose should be | endoscopy) and throughout the duration |
| | discontinued. | of the study. |
| | Oral prednisone ≤20 mg/day (or dose | IV corticosteroids and rectal |
| | equivalent of other oral corticosteroids, e.g., | corticosteroids (i.e., enemas or |
| | oral budesonide MMX ≤9 mg/day) if on | suppositories) ≤2 weeks prior to |
| | stable dose for ≥2 weeks prior to screening | screening endoscopy (or SOC endoscopy) |
| | endoscopy (or SOC endoscopy). Stable | and throughout the duration of the study, |
| | dose must continue through the induction | with the exception of a single |
| | phase. Oral corticosteroid tapering must be | administration of IV steroid for potential |
| | initiated at Week 12 (GA45331 study) or | IRR management. |
| | within the first 12 weeks of entering the OLE | Initiation of oral corticosteroids from |
| | phase (GA45332 study). | randomization (Week 0, Day 1) and |
| | Oral prednisone ≥20 mg/day allowed at | throughout the duration of the study, with |
| | screening if tapered down to a stable dose | the exception for the treatment of an |
| | of ≤20 mg/day by 2 weeks prior to screening | adverse event (up to 20 mg/day, or dose |
| | endoscopy (or SOC endoscopy). | equivalent of other oral corticosteroids). |
| Immuno- | AZA, 6-MP, or MTX if on stable dose 8 | Cyclosporine, thalidomide, |
| suppressants | weeks prior to randomization (Week 0, | tacrolimus, sirolimus or |
| | Day 1) and continue stable dosing | mycophenolate mofetil ≤16 weeks |
| | throughout the duration of the study; | prior to randomization (Week 0, Day 1) |
| | discontinuation of concomitant | and throughout the duration of the |
| | medication permitted if the dose is | study. |
| | reduced or discontinued due to toxicity. | Any immunosuppressive therapy |
| | | including B-cell depleting agents |
| | | (e.g., natalizumab, rituximab) or |
| | | other lymphocyte depleting agents |
| | | (e.g., alemtuzumab), or any other |
| | | immunosuppressive agents (e.g., |
| | | those where live vaccines are |
| | | contraindicated) within 1 year prior to |
| | | screening and during the study. |
| Advanced | N/A | Small molecule (JAK inhibitor) ≤2 |
| Therapies | | weeks prior to screening endoscopy |
| | | (or SOC endoscopy) and throughout |
| | | the duration of the study. |
| | | Any newly approved S1P receptor |
| | | modulators ≤2 weeks prior to |
| | | screening endoscopy (or SOC |
| | | endoscopy) and throughout the |
| | | duration of the study. |
| | | Biologics (anti-TNF, anti-integrin, |
| | | and anti-interleukin including |
| | | biosimilars) within 8 weeks or 5 |
| | | half-lives prior to randomization |
| | | (Week 0, Day 1), whichever is |
| | | shorter, and throughout the |
| | | duration of the study. |
| | | If there is proper documentation of |
| | | undetectable drug level measured by |
| | | a commercially available assay for |
| | | any of the approved biologics, there |
| | | is no minimum washout prior to |
| | | randomization (Week 0, Day 1). |
| | | Use of any investigational or |
| | | experimental therapy within |
| | | approximately 30 days for non- |
| | | biologic therapy or 8 weeks for |
| | | biologic therapy OR 5 half-lives |
| | | (whichever is shorter) prior to |
| | | randomization (Week 0, Day 1). |
| Oral probiotic | Oral probiotics if dose is stable ≥2 weeks | Initiation or dose change throughout |
| therapies | prior to screening endoscopy (or SOC | the duration of the study. |
| | endoscopy) and continue stable dosing | |
| | throughout the duration of the study. | |
| Other | CD-related antibiotics (e.g., ciprofloxacin or | Initiation of CD-related antibiotics |
| Therapies | metronidazole) if dose is stable ≥2 weeks | (oral or IV) throughout the duration |
| | prior to screening endoscopy (or SOC | of study, with the exception of those |
| | endoscopy)and up to randomization | for the treatment of fistulas. |
| | (Week 0, Day 1). | Anti-TL1A agents at any time prior |
| | | to or during the study (except |
| | | afimkibart administered as study |
| | | treatment in this study). |
| | | Continued tube feeding and/or total |
| | | parenteral alimentation/nutrition as |
| | | treatment for CD ≤3 weeks prior to |
| | | randomization or during the study. |
| | | Treatment with approved CD oral |
| | | traditional medicine (e.g., Chinese |
| | | medicine) ≤4 weeks prior to |
| | | screening endoscopy (or SOC |
| | | endoscopy) and throughout the |
| | | study. |

5-ASA = 5-aminosalicylic acid; 6-MP = 6-mercaptopurine; AZA = azathioprine; CD = Crohn's disease; IRR = infusion-related reaction; JAK = Janus kinase; MTX = methotrexate; NA = not applicable; TL1A = tumor necrosis factor-like ligand 1A; TNF = tumor necrosis factor; S1P = sphingosine-1-phosphate.
Note:
During the OLE phase, oral aminosalicylates, immunomodulators, oral corticosteroids, CD-related antibiotics, rectal or systemic corticosteroids for treatment of CD may be initiated, stopped, or the dose may be adjusted at the investigator's discretion.

TABLE 30
Permitted and Prohibited Concomitant Therapies Not Related to Crohn's Disease

| Permitted | Prohibited |
|---|---|
| Occasional use of non-steroid | Any investigational or experimental therapy within 30 days for |
| anti-inflammatory drugs | non-biologic therapy or 8 weeks for biologic therapy, or |
| (NSAIDs), acetaminophen, and | 5 half-lives prior to randomization (Week 0, Day 1), |
| aspirin ≤325 mg/day. | whichever is shorter. Note: If there is proper |
| Non-live (inactivated) | documentation of undetectable drug level measured by a |
| vaccines. | commercially available assay for any of the approved |
| CYP substrates with a narrow | biologics, there is no minimum washout prior to |
| therapeutic window, including | randomization (Week 0, Day 1)and throughout the study. |
| but not | Any immunosuppressive therapy including B-cell depleting |
| limited to aminoglycosides, | agents (e.g., natalizumab, rituximab) or other lymphocyte |
| ciclosporin, carbamazepine, | depleting agents (e.g., alemtuzumab), or any other |
| digoxin, digitoxin, flecainide, | immunosuppressive agents (e.g., those where live vaccines |
| lithium, phenytoin, | are contraindicated) within 1 year prior to screening and |
| phenobarbital, rifampicin, | throughout the study. |
| theophylline, and warfarin. | Topical rectal traditional medicine (e.g., Chinese medicine), |
| Monitoring the effect of drug | herbal enemas, or suppositories ≤2 weeks prior to |
| concentration of the | screening endoscopy (or SOC endoscopy) and throughout |
| concomitant treatment (on | the study. |
| initiation or discontinuation of | Fecal microbial transplantation ≤4 weeks prior to |
| afimkibart) is strongly | randomization (Week 0, Day 1). |
| recommended. | Live or attenuated vaccines ≤4 weeks prior to screening |
| | endoscopy (or SOC endoscopy) and throughout the study. |
| | IV antibiotics ≤4 weeks prior to randomization (Week 0, Day 1). |
| | Apheresis ≤2 weeks prior to screening endoscopy (or SOC |
| | endoscopy) and throughout the study. |
| | Immunoglobulin or blood products ≤4 weeks prior to |
| | screening endoscopy (or SOC endoscopy), or any |
| | condition that is likely to require such treatment |
| | during the course of the study. |
| | Transplant/stem cell therapy at any time prior to |
| | randomization (Week 0, Day 1) and throughout the study. |
| | Chronic (e.g., >7 days) use of NSAIDs, acetaminophen, |
| | and/or aspirin >325 mg/day during the study. |

### B. Corticosteroid Tapering

In the GA45331 and GA45332 studies, during the 12-week induction phase, participants are to maintain their stable baseline corticosteroid dose.

In the GA45331 study, following the Week 12 assessment, participants begin corticosteroid tapering during the maintenance phase.

In the GA45332 study, following the Week 12 assessment, and at the investigator's discretion, participants begin corticosteroid tapering within the first 12 weeks of entering the OLE.

The recommended tapering schedule for oral corticosteroids is shown in Table 31.

TABLE 31
Tapering Schedule for Oral Corticosteroids

| Corticosteroid | Dose | | Tapering Rate |
|---|---|---|---|
| Oral prednisone or | >10 | mg/day | Taper daily dose by 5 mg/week until receiving 10 |
| equivalent | | | mg/day, and then continue tapering at 2.5 |
| | | | mg/week until 0 mg/day. |
| | ≤10 | mg/day | Taper daily dose by 2.5 mg/week until 0 mg/day. |
| Oral budesonide | ≤9 | mg/day | Taper tablets to 9 mg every other day for |
| | | | 2 weeks, followed by 9 mg every third day for 2 |
| | | | weeks, and then discontinue. |

For participants who cannot tolerate the corticosteroid taper without recurrence of CD clinical symptoms or steroid withdrawal, the corticosteroid dose may be increased (up to the dose at trial entry if required) per the investigator's discretion during the study, but tapering should begin again within 2 weeks.

### C. Efficacy Assessments

Patient-reported outcome (PRO) and clinician-reported outcome (ClinRO) instruments are completed to assess the treatment benefit of afimkibart. PRO data are collected through the instruments summarized in Table 32 and described below.

TABLE 32
Patient-Reported Outcome Instruments

| PRO Instrument | Recall Period | |
|---|---|---|
| Stool frequency | 24 | hours |
| Abdominal pain | | |
| General well-being | | |
| Bowel urgency | | |
| Functional Assessment of Chronic Illness Therapy-Fatigue | 7 | days |
| (FACIT-F) | | |
| Inflammatory Bowel Disease Questionnaire (IBDQ) | 2 | weeks |
| Patient Global Impression of Change (PGIC) | Current versus baseline | |
| Patient Global Impression of Severity (PGIS) | 7 | days |
| EuroQol 5-Dimension 5-Level (EQ-5D-5L) (used to evaluate health | Current | |
| economics) | | |
| Work Productivity and Activity Impairment Questionnaire (WPAI- | 7 | days |
| CD) (used to evaluate health economics) | | |

PRO instruments are collected by eDiary or during visits to clinic or by mobile nursing. In general, eDiary PROs are to be completed daily during screening, induction and maintenance. During the OLE phase, participants are to complete the eDiary PROs for at least 7 days prior to each quarterly (Q3M) visit during the first year and prior to the annual visit thereafter. If disease worsening criteria assessment is required in the OLE phase, eDiary PROs are to be collected for at least 3 weeks prior to the visit at which the assessment is being conducted. For all phases of the study, eDiary entries are not required the day the participant receives medication for bowel preparation prior to endoscopy and the day the participant undergoes an endoscopy.

ClinRO data are collected, in part, through the CDAI. Additional ClinRO data are evaluated through central endoscopy and histology assessments by the Simple Endoscopic Score for Crohn's Disease (SES-CD) and the Geboes Grading Scale, respectively.

i. Crohn's Disease Activity Index

Crohn's Disease Activity Index (CDAI) (Best et al., Gastroenterology, 70 (3): 439-444, 1976) is evaluated at specified timepoints to quantify signs and symptoms of CD. This index is a weighted sum of scores on eight components: number of liquid or soft stools (stool frequency), abdominal pain, general well-being, number of complications, use of anti-diarrheal medication, presence of an abdominal mass, hematocrit, and percentage deviation from standard body weight. Stool frequency, abdominal pain and general well-being components are PROs reported via eDiary. Remaining CDAI components are evaluated by physician or laboratory assessment. CDAI generally ranges from 0 to roughly 600, with higher values indicating greater activity.

ii. Stool Frequency, Abdominal Pain and General Well-Being

Each PRO component of CDAI contributes a summary (e.g., a sum or average) of daily eDiary entries over 7 days prior to the relevant timepoint. These weekly summaries are further used to evaluate disease worsening criteria and endpoints.

iii. Endoscopy

All participants undergo an ileocolonoscopy at specified visits or as part of SOC (if done before the screening period, independently of the study). After Week 12, additional endoscopies at unscheduled visits may be necessary in order to facilitate the evaluation of disease worsening criteria, which determines OLE eligibility or dose intensification in OLE. The Simple Endoscopic Score for Crohn's Disease (SES-CD) is a composite of four features of endoscopic activity (presence and size of ulcers, extent of ulcerated surface, extent of affected and presence and type of narrowings or stenosis) in up to five ileocolonic segments (terminal ileum, right colon, transverse colon, sigmoid and left colon, and rectum). Each feature is scored on a scale from 0 to 3, giving segment subscores of 0 to 12 points and a total SES-CD range of 0-60, with a higher value indicating greater severity.

iv. Geboes Grading Scale

Biopsy specimens collected during ileocolonoscopy are used to evaluate histologic activity per centrally-read Geboes Grading Scale (Geboes et al., Gut, 47:404-409, 2000). This scale is a seven-item classification system to evaluate histological activity originally developed for UC, with items graded from least to most severe features of activity. Each grade is assigned a subgrade ranging from 0 to 3 or 4, with higher values associated with greater severity of the corresponding feature. Although originally developed for UC, the Geboes scale has been previously used to evaluate histologic activity in CD and its use in CD is endorsed by expert consensus.

v. Bowel Urgency

Bowel urgency is a single-item self-reported assessment of sudden or immediate need to have a bowel movement in the past 24 hours. The item response is reported on a 4-point Likert scale, from “None” to “Severe.”

vi. FACIT-F

The Functional Assessment of Chronic Illness-Fatigue (FACIT-F; Version 4) is a 13-item self-reported assessment of fatigue. FACIT-F has been validated for use in a variety of conditions, including anemia and IBD (Yellen et al., J Pain Symptom Manage, 13:63-74, 1997; Cella et al., J Rheum, 32:811-819, 2005; Lai et al., J Rheumatol, 38:672-679, 2011; Tinsley et al., Aliment Pharmacol Ther, 34:1328-1336, 2011; Acaster et al., Health Qual Life Outcomes, 13:60, 2015). Each item response option indicates the degree to which a given statement describing the level or impact of fatigue applies in the past 7 days. Response options are graded on a 5-point Likert-type scale, from “Not at all” to “Very much.”

vii. IBDQ

The Inflammatory Bowel Disease Questionnaire (IBDQ) is a validated self-reported 32-item assessment of health-related quality of life (QoL) in patients with IBD (Guyatt et al., Gastroenterology, 96:80410, 1989; Irvine, J Pediatr Gastroenterol Nutr, 28: S23-S27, 1999). IBDQ covers four domains: bowel symptoms (10 questions); systemic symptoms including sleep disorders and fatigue (5 questions); emotional function such as depression, aggression, and irritation (12 questions); and social function, meaning the ability to participate in social activities and work (5 questions). Each question has a recall period of the past 2 weeks. Response options are graded on a 7-point Likert-type scale.

viii. PGIC

The Patient Global Impression of Change (PGIC) is a single-item self-reported assessment of the change in overall CD symptoms, from the start of the study to current status. The item response is reported on a 5-point Likert-type scale, from “Much worse” to “Much better.”

ix. PGIS

The Patient Global Impression of Severity (PGIS) is a single-item self-reported assessment of the severity of overall CD symptoms. The item response is reported on a 6-point Likert scale, from “None” to “Very severe.”

x. Fistula Examination

Fistula examinations are performed only on participants presenting with actively draining fistulas at baseline (Week 0, Day 1). Fistulas are assessed for draining or closed status, where closed fistulas are assessed by the investigator as no longer draining despite gentle finger compression. Setons (placed previously or placed during the study per standard of care) are allowed. Seton removal is also conducted per local standard of care.

### Example 11. CD Study Statistical Considerations

This Example provides a summary of the statistical aspects of the GA45331 and GA45332 study designs and the general approach to analysis of endpoints supporting the study objectives. Analysis of study data are anchored upon the following milestones:
Primary completion, given by the date at which the last participant in the maintenance treatment phase completes all Week 52 primary endpoint assessments (for the GA45331 study) or the date at which the last participant in the induction treatment phase completes all Week 12 primary endpoint assessments (for the GA45332 study).
Study completion, given by the date of the last-participant, last-visit across all phases of the study, including OLE phase.

### A. Statistical Hypotheses

Under the co-primary objectives for the GA45331 study, it is hypothesized that afimkibart is superior to placebo in inducing and maintaining clinical remission and endoscopic response.

Under the primary objective for the GA45332 study, it is hypothesized that afimkibart is superior to placebo in inducing clinical remission and endoscopic response.

In statistical testing, the relevant null and alternative hypotheses are formulated as follows:

H₀: p_{afimkibart}−p_{placebo}=0 versus H_{A}: p_{afimkibart}−p_{placebo}≠0,

where p_{afimkibart}−p_{placebo} represents the treatment effect (estimand) of interest, the difference in the proportion of patients achieving clinical remission at a particular timepoint (Week 12 or 52) after assignment to afimkibart versus placebo, as described in Example 1.

The GA45331 study is deemed positive if all null hypotheses based on clinical remission and endoscopic response at Week 12 and at Week 52 are rejected in favor of afimkibart IV induction followed by 450 mg SC maintenance versus placebo.

The GA45332 study is deemed positive if all null hypotheses based on clinical remission and endoscopic response at Week 12 are rejected in favor of afimkibart.

### B. Sample Size Determination

In the GA45331 study, a total of approximately 600 participants are enrolled and randomly assigned to either afimkibart with a 450 mg or 150 mg maintenance dose or placebo under a 1:1:1 randomization ratio. This allocates approximately 200 participants to each of the three treatment arms. In the GA45332 study, a total of approximately 425 participants are enrolled and randomly assigned to either afimkibart or placebo under a 3:2 randomization ratio. This allocates approximately 255 participants to the afimkibart arms and 170 to the placebo arms.

Marginal power to reject one of the null hypotheses in primary analysis at the significance level of 5% depends (in part) on the clinical remission rate under placebo treatment. In a meta-analysis of recent Phase II and III CD trials, placebo induction of clinical remission and endoscopic response were estimated to be 18% (95% CI: 16% to 21%) and 13% (95% CI: 10% to 16%), respectively. In a meta-analysis of randomized withdrawal trials, maintenance of clinical remission and endoscopic response were estimated to be 44% (95% CI: 26% to 63%; Almradi et al., BioDrugs, 34:713-721, 2020) and 7% (95% CI: 1% to 31%; Vuyyuru et al., Inflamm Bowel Dis, 30:651-659, 2024), respectively. Placebo rates in general vary according to a variety of factors, such as the proportion of participants with prior advanced therapy (Almradi et al., J Crohns Colitis, 16:717-736, 2022; Vuyyuru et al., Inflamm Bowel Dis, 30:651-659, 2024).

Under a treat-through study design, where participants enter maintenance without necessarily achieving clinical or symptomatic response and remain on either experimental or placebo treatment, it is anticipated that the placebo maintenance outcome rates will be no greater than the rate at induction and lower than rates observed in randomized withdrawal, provided that intercurrent events are appropriately addressed. This phenomenon has been observed in recent Phase III trials (VIVID-1 and GALAXI 2 and 3) that follow a treat-through design except in placebo induction non-responders, who receive rescue with either experimental or active control therapy. Considering the provision of rescue as treatment failure (as one would under a fully treat-though design), the induction versus maintenance placebo outcome rates were 25% vs 20% for clinical remission and 13% vs 9% for endoscopic response in VIVID-1 (Ferrante et al., J Crohns Colitis, 18 (Supplement 1): 17-9, 2024) and 19% vs 12% for clinical remission and 12% vs 5% for endoscopic response in GALAXI 2 and 3 (Sands et al., Am J Gasteroenterol, 119 (10S): S1039, 2024).

### Example 12. Modeling Data Supporting the Induction Phase Dosing Regimen

### A. Previous Clinical Trials

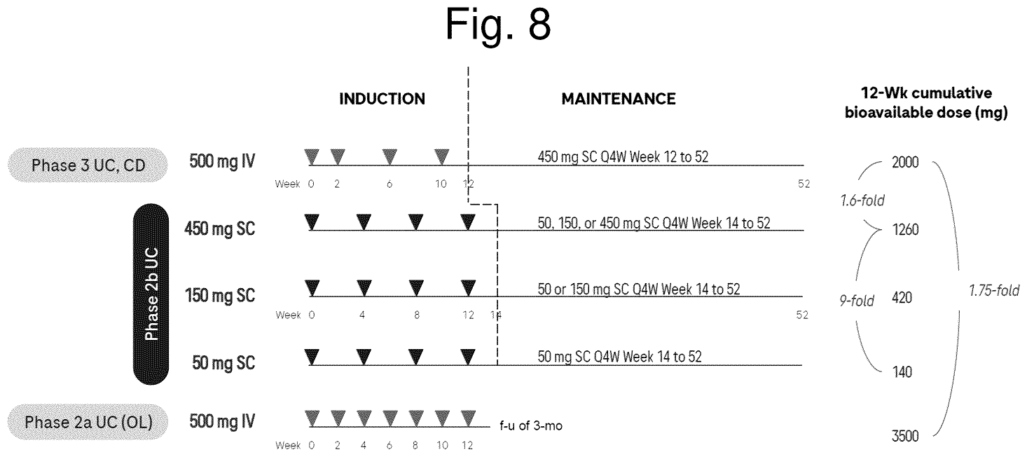

FIG. 8 shows a comparison between the GA45329 (ulcerative colitis (UC)) and GA45331 (Crohn's disease (CD)) studies provided in Examples 1-11 and two previous clinical trials in which afimkibart was administered to subjects having UC.

Afimkibart has previously been shown to be effective and well tolerated, with a favorable benefit/risk profile, in the phase IIa TUSCANY trial and the subsequent phase IIb TUSCANY-2 trial in patients with moderately to severely active UC. Briefly, in the Phase 2a study (TUSCANY; NCT02840721), subjects received afimkibart intravenously (IV) every two weeks at a dose of 500 mg over a twelve-week period (induction phase), for a total of seven doses.

In the induction phase of the Phase 2b study (TUSCANY-2; NCT04090411), subjects received afimkibart subcutaneously (SC) every four weeks at a dose of 50 mg, 150 mg, or 450 mg over a twelve-week period, for a total of four induction phase doses.

As described in Examples 1-11, above, in the induction phase of the Phase 3 trial, patients receive afimkibart IV at a dose of 500 mg at Weeks 0, 2, 6, and 10 of the induction phase, for a total of four induction phase doses.

FIG. 8 shows the calculated cumulative bioavailable dose of afimkibart at 12 weeks (i.e., following the induction phase) in each trial. Bioavailable doses in the SC treatment arms have been calculated in accordance with standard practice (70% of the administered dose). In the Phase 3 trial, the calculated cumulative bioavailable dose is 2000 mg. This bioavailable dose is 1.75-fold lower than the bioavailable dose reached in the Phase 2a study, and is 1.6-fold higher than the bioavailable dose reached by the 450 mg treatment arm in the Phase 2b study.

### B. Modeling Data

The induction phase dosing regimen for the Phase 3 study provided in Examples 1-11 was designed to increase the likelihood of maximal blockade of membrane TL1A and maximal suppression of soluble TL1A in inflamed tissues in patients having conditions that show higher TL1A expression in the gut (such as IBD, e.g., UC or CD).

Historical evidence relating to biologics in inflammatory bowel disease (IBD) indicates that a higher drug concentration (PK) during the induction phase is associated with better and more sustained clinical outcomes. These historical data also highlight the importance of target saturation at inflamed tissue levels, beyond serological levels, for maximal drug efficacy.

The Phase 3 induction phase dosing regimens provided herein provide (1) a high bioavailable dose during induction (see FIG. 8) and (2) a “loading approach” in which the first and second doses are administered two weeks apart. This approach increases the likelihood of early onset of action; historical data demonstrate that early onset translates into improved maintenance efficacy.

In addition, the induction phase dosing regimen for the Phase 3 studies provided in Examples 1-11 was designed to minimize any potential impact of immunogenicity on PK, efficacy, and safety. In particular, an IV route of administration was selected (e.g., over SC route of administration) to minimize immunogenicity.

Relative to the Phase 2b (SC) induction phase dosing regimens, the Phase 2a (IV) induction phase dosing regimen was associated with lower anti-drug antibody (ADA) titers, lower maximum titers, and delayed median time to first ADA and first neutralizing antibody (NAb). The median times to first detection of ADAs and NAbs were 140 and 114 days, respectively, with the Phase 2a 500 mg IV dosing regimen, and were 30-57 days, and 58-85 days, respectively, during induction with the Phase 2b SC dosing regimens. For the IV regimen, similarity of PK profiles (ADA-positive patients vs. ADA-negative patients) was observed, and no statistically significant effect of ADA/NAb status was observed on endoscopic improvement, remission, or endoscopic remission at Week 14. Further, in the Phase 2b SC induction phase dosing regimens, higher ADA titers were associated with lower exposure after SC administration.

### C. Modeling of the Phase 3 Induction Phase

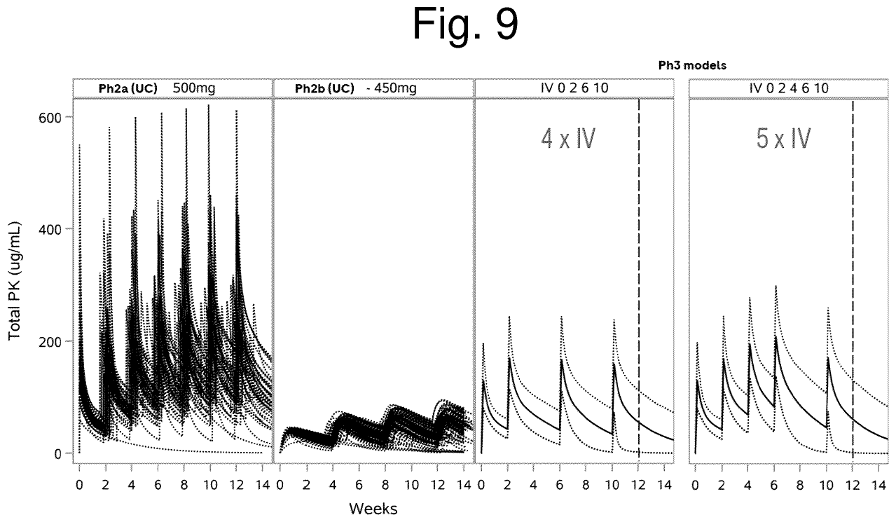

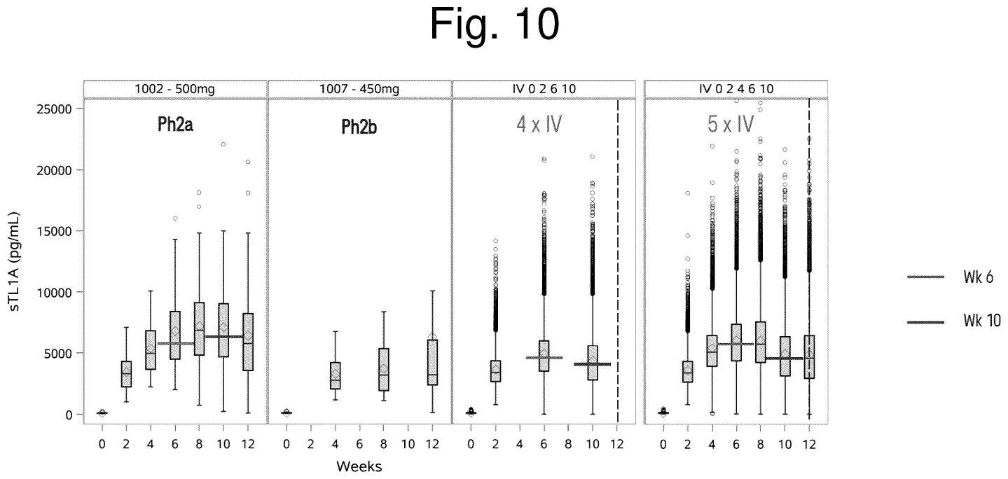

FIGS. 9 and 10 present modeled PK and soluble TL1A (sTL1A) data for two induction phase dosing regimens for the Phase 3 trial, as compared to real data from blood samples collected during the induction phases of the Phase 2a and Phase 2b studies.

In the contemplated Phase 3 “4×IV” induction phase regimen, afimkibart is administered IV at a dose of 500 mg at Weeks 0, 2, 6, and 10 of a 12-week induction phase dosing regimen, for a total of four doses. In the contemplated Phase 3 “5×IV” induction phase regimen, afimkibart is administered IV at a dose of 500 mg at Weeks 0, 2, 4, 6, and 10 of a 12-week induction phase dosing regimen, for a total of five doses.

The models were constructed using data from the Phase 2a and 2b studies. For the PK model (FIG. 9), the effect of ADAs on the elimination of afimkibart was factored into the model (overall lower ADA titers in IV as compared to SC dosing). Similarly, for the sTL1A model (reflecting patient experience during treatment) (FIG. 10), the effect of ADAs on the elimination of drug-target complexes was factored in. Good correlation between predicted and observed profiles was observed for both models.

These modeling data indicate that from the perspectives of PK and patient experience, the 4×IV regimen was not substantially less favorable than the 5×IV regimen. Therefore, the 4×IV regimen was selected, at least in part, on the basis of its greater convenience for patients and medical practitioners.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating an inflammatory bowel disease (IBD) in a patient, the method comprising administering to the patient an effective amount of an anti-TNF-like ligand 1A (TL1A) antibody in a dosing regimen comprising an induction phase, wherein the induction phase comprises administration of only four doses of the anti-TL1A antibody, wherein:
(i) the second dose of the anti-TL1A antibody is administered about two weeks after the first dose;
(ii) the third dose of the anti-TL1A antibody is administered about four weeks after the second dose; and
(iii) the fourth dose of the anti-TL1A antibody is administered about four weeks after the third dose,
wherein the anti-TL1A antibody comprises the following complementarity-determining regions (CDRs):
(a) a CDR-H1 comprising the amino acid sequence of SEQ ID NO: 3;
(b) a CDR-H2 comprising the amino acid sequence of SEQ ID NO: 4;
(c) a CDR-H3 comprising the amino acid sequence of SEQ ID NO: 5;
(d) a CDR-L1 comprising the amino acid sequence of SEQ ID NO: 6;
(e) a CDR-L2 comprising the amino acid sequence of SEQ ID NO: 7; and
(f) a CDR-L3 comprising the amino acid sequence of SEQ ID NO: 8.

2. The method of claim 1, further comprising administering to the patient an effective amount of the anti-TL1A antibody in a maintenance phase after the induction phase, wherein the maintenance phase comprises administration of the anti-TL1A antibody every four weeks.

3. The method of claim 1, wherein, in the induction phase, the anti-TL1A antibody is administered intravenously at a dose of 500 mg.

4. The method of claim 1, wherein, in the induction phase, the anti-TL1A antibody is administered subcutaneously at a dose of 500 mg.

5. The method of claim 1, wherein the induction phase has a duration of 12 weeks, and wherein:
(a) the first dose of the anti-TL1A antibody is administered on Day 1 of Week 0;
(b) the second dose of the anti-TL1A antibody is administered on Day 1 of Week 2;
(c) the third dose of the anti-TL1A antibody is administered on Day 1 of Week 6; and
(d) the fourth dose of the anti-TL1A antibody is administered on Day 1 of Week 10.

6-7. (canceled)

8. The method of claim 2, wherein, in the maintenance phase, the anti-TL1A antibody is administered subcutaneously at a dose of 150 mg.

9. The method of claim 2, wherein, in the maintenance phase, the anti-TL1A antibody is administered subcutaneously at a dose of 450 mg.

10. (canceled)

11. The method of claim 2, wherein the maintenance phase comprises:
(a) subcutaneous administration of the anti-TL1A antibody every four weeks;
(b) subcutaneous administration of the anti-TL1A antibody every two weeks; or
(c) (i) at least one interval in which the anti-TL1A antibody is administered every four weeks and (ii) at least one interval in which the anti-TL1A antibody is administered every two weeks.

12-21. (canceled)

22. The method of claim 2, wherein the first dose of the maintenance phase is administered two weeks after administration of the fourth dose of the induction phase.

23. The method of claim 2, wherein the dosing regimen has a duration of 52 weeks, and wherein:
(a) the first dose of the induction phase is administered on Day 1 of Week 0;
(b) the second dose of the induction phase is administered on Day 1 of Week 2;
(c) the third dose of the induction phase is administered on Day 1 of Week 6;
(d) the fourth dose of the induction phase is administered on Day 1 of Week 10:
(e) the first dose of the maintenance phase is administered on Day 1 of Week 12; and
(f) the subsequent doses of the maintenance phase are administered on Day 1 of Weeks 16, 20, 24, 28, 32, 36, 40, 44, 48, and 52.

24-27. (canceled)

28. The method of claim 1, wherein the IBD is ulcerative colitis (UC).

29. The method of claim 28, wherein:
(i) the UC is moderately to severely active ulcerative colitis;
(ii) the patient has a modified Mayo score (mMS) of between 5 points and 9 points; or
(iii) the patient has a Mayo endoscopic score(ES) of 2 or 3.

30-31. (canceled)

32. The method of claim 1, wherein the IBD is Crohn's disease (CD).

33. The method of claim 32, wherein:
(i) the CD is moderately to severely active CD;
(ii)
(a) the patient has a Simple Endoscopic Score for Crohn's Disease (SES-CD) of equal to or greater than 6; or
(b) the patient has isolated ileal disease only, and has an SES-CD of equal to or greater than 4; or
(iii) the patient has a Crohn's disease activity index (CDAI) that is at least 220 and is no greater than 450.

34-35. (canceled)

36. The method of claim 1, wherein the patient has previously been treated with a therapy for UC or CD and has experienced inadequate response to the therapy, loss of response to the therapy, and/or intolerance of the therapy.

37-40. (canceled)

41. A method of treating ulcerative colitis (UC) in a patient, the method comprising administering to the patient an effective amount of an anti-TNF-like ligand 1A (TL1A) antibody in a dosing regimen comprising an induction phase and a maintenance phase, wherein:
(a) the induction phase comprises intravenous administration of only four doses of the anti-TL1A antibody at a dose of 500 mg, wherein:
(i) the second dose of the anti-TL1A antibody is administered about two weeks after the first dose;
(ii) the third dose of the anti-TL1A antibody is administered about four weeks after the second dose; and
(iii) the fourth dose of the anti-TL1A antibody is administered about four weeks after the third dose; and
(b) the maintenance phase comprises subcutaneous administration of the anti-TL1A antibody every four weeks at a dose of 450 mg,
wherein the anti-TL1A antibody comprises the following CDRs:
(a) a CDR-H1 comprising the amino acid sequence of SEQ ID NO: 3;
(b) a CDR-H2 comprising the amino acid sequence of SEQ ID NO: 4;
(c) a CDR-H3 comprising the amino acid sequence of SEQ ID NO: 5;
(d) a CDR-L1 comprising the amino acid sequence of SEQ ID NO: 6;
(e) a CDR-L2 comprising the amino acid sequence of SEQ ID NO: 7; and
(f) a CDR-L3 comprising the amino acid sequence of SEQ ID NO: 8.

42-46. (canceled)

47. A method of treating Crohn's disease (CD) in a patient, the method comprising administering to the patient an effective amount of an anti-TNF-like ligand 1A (TL1A) antibody in a dosing regimen comprising an induction phase and a maintenance phase, wherein:
(a) the induction phase comprises intravenous administration of only four doses of the anti-TL1A antibody at a dose of 500 mg, wherein:
(i) the second dose of the anti-TL1A antibody is administered two weeks after the first dose;
(ii) the third dose of the anti-TL1A antibody is administered four weeks after the second dose; and
(iii) the fourth dose of the anti-TL1A antibody is administered four weeks after the third dose; and
(b) the maintenance phase comprises subcutaneous administration of the anti-TL1A antibody every four weeks at a dose of 150 mg or 450 mg,
wherein the anti-TL1A antibody comprises the following CDRs:
(a) a CDR-H1 comprising the amino acid sequence of SEQ ID NO: 3;
(b) a CDR-H2 comprising the amino acid sequence of SEQ ID NO: 4;
(c) a CDR-H3 comprising the amino acid sequence of SEQ ID NO: 5;
(d) a CDR-L1 comprising the amino acid sequence of SEQ ID NO: 6;
(e) a CDR-L2 comprising the amino acid sequence of SEQ ID NO: 7; and
(f) a CDR-L3 comprising the amino acid sequence of SEQ ID NO: 8.

48-61. (canceled)

62. The method of claim 1, wherein the anti-TL1A antibody comprises:
(a) a heavy chain variable (VH) domain comprising an amino acid sequence having at least 95% sequence identity to the amino acid sequence of SEQ ID NO: 1; and/or
(b) a light chain variable (VL) domain comprising an amino acid sequence having at least 95% sequence identity to the amino acid sequence of SEQ ID NO: 2.

63-64. (canceled)

65. The method of claim 1, wherein the anti-TL1A antibody comprises:
(a) a heavy chain comprising an amino acid sequence having at least 95% sequence identity to the amino acid sequence of SEQ ID NO: 9 or SEQ ID NO: 11; and/or
(b) a light chain comprising an amino acid sequence having at least 95% sequence identity to the amino acid sequence of SEQ ID NO: 10.

66-67. (canceled)

68. The method of claim 1, wherein the anti-TL1A antibody is afimkibart.

69-91. (canceled)
